(19) United States
(12) Patent Application Publication
(10) Pub. No.: US 2020/0017600 A1
(43) Pub. Date: Jan. 16, 2020

(54) VARIANTS OF CD38 ANTIBODY AND USES THEREOF

(71) Applicant: GENMAB A/S, Copenhagen V (DK)

(72) Inventors: Bart De GOEIJ, Utrecht (NL); Grietje ANDRINGA, Utrecht (NL); Frank BEURSKENS, Utrecht (NL); Janine SCHUURMAN, Diemen (NL); David P.E. SATIJN, Utrecht (NL); Tahamtan AHMADI, Rydal, PA (US)

(21) Appl. No.: 16/512,206

(22) Filed: Jul. 15, 2019

Related U.S. Application Data
(60) Provisional application No. 62/848,874, filed on May 16, 2019.
(60) Provisional application No. 62/697,730, filed on Jul. 13, 2018.

(51) Int. Cl. C07K 16/28; A61P 35/00

(52) U.S. Cl. CPC C07K 16/2896; A61P 35/00; C07K 2317/52; C07K 2317/21; A61K 2039/505; C07K 2317/732; C07K 2317/92; C07K 2317/73; C07K 2317/734

(57) ABSTRACT

Antibody variants comprising one or more mutations in the Fc region, particularly anti-CD38 antibodies comprising a mutation in one or more amino acid residues corresponding to E430, E345 and S440 in a human IgG1 heavy chain, wherein the amino acid residues are numbered according to the EU index.

### CROSS-REFERENCE TO RELATED APPLICATIONS

This application claims priority to U.S. Provisional Application Nos. 62/697,730, and 62/848,874, filed on Jul. 13, 2018, and May 16, 2019, respectively. The contents of the aforementioned applications are hereby incorporated by reference.

### SEQUENCE LISTING

The instant application contains a Sequence Listing which has been submitted via EFS-Web and is hereby incorporated by reference in its entirety. Said ASCII copy, created on Jul. 15, 2019, is named GMI_172_Sequence_Listing.txt and is 77,432 bytes in size.

### FIELD OF THE INVENTION

Antibody variants comprising one or more mutations in the Fc region, particularly anti-CD38 antibody variants.

### BACKGROUND OF THE INVENTION

CD38 is a type II transmembrane glycoprotein which is normally found on hematopoietic cells and at low levels in solid tissues. Expression of CD38 in hematopoietic cells depends on the differentiation and activation status of the cell. Lineage-committed hematopoietic cells express the protein, while it is lost by mature cells and expressed again on activated lymphocytes. CD38 is also expressed on B cells, whereby plasma cells express particularly high levels of CD38. Approximately 80% of resting NK cells and monocytes express CD38 at lower levels, as do various other hematological cell types, including lymph node germinal center lymphoblasts, intrafollicular cells, dendritic cells, erythrocytes, and platelets (Lee and Aarhus 1993; Zocchi, Franco et al. 1993; Malavasi, Funaro et al. 1994; Ramaschi, Torti et al. 1996). With regard to solid tissues, CD38 is expressed in the gut by intraepithelial cells and lamina propria lymphocytes, by Purkinje cells and neurofibrillary tangles in the brain, by epithelial cells in the prostate, β-cells in the pancreas, osteoclasts in the bone, retinal cells in the eye, and sarcolemma of smooth and striated muscle.

CD38 is expressed in a large number of hematological malignancies. Expression has been observed particularly in the malignant cells of multiple myeloma (MM) (Lin, Owens et al. 2004) and chronic lymphocytic leukemia (CLL) (Damle 1999), and was also reported in WaldenstrOm's macroglobulinemia (Konoplev, Medeiros et al. 2005), primary systemic amyloidosis (Perfetti, Bellotti et al. 1994), mantle-cell lymphoma (Parry-Jones, Matutes et al. 2007), acute lymphoblastic leukemia (Keyhani, Huh et al. 2000), acute myeloid leukemia (Marinov, Koubek et al. 1993; Keyhani, Huh et al. 2000), NK-cell leukemia (Suzuki, Suzumiya et al. 2004), NK/T-cell lymphoma (Wang, Wang et al. 2015) and plasma cell leukemia (van de Donk, Lokhorst et al. 2012).

Other diseases, where CD38 expression could be involved, include, e.g. broncho-epithelial carcinomas of the lung, breast cancer (evolving from malignant proliferation of epithelial lining in ducts and lobules of the breast), pancreatic tumors, evolving from the β-cells (insulinomas), tumors evolving from epithelium in the gut (e.g. adenocarcinoma and squamous cell carcinoma), carcinoma in the prostate gland, seminomas in testis, ovarian cancers, and neuroblastomas. Other disclosures also suggest a role of CD38 in autoimmunity such as Graves disease and thyroiditis (Antonelli, Fallahi et al. 2001), type 1 and 2 Diabetes (Mallone and Perin 2006) and inflammation of airway smooth muscle cells during asthma (Deshpande, White et al. 2005). Moreover, CD38 expression has been associated with HIV infection (Kestens, Vanham et al. 1992; Ho, Hultin et al. 1993).

CD38 is a multifunctional protein. Functions ascribed to CD38 include both receptor mediation in adhesion and signaling events and (ecto-) enzymatic activity. As an ectoenzyme, CD38 uses NAD⁺ as substrate for the formation of cyclic ADP-ribose (cADPR) and ADPR, but also of nicotinamide and nicotinic acid-adenine dinucleotide phosphate (NAADP). cADPR has been shown to act as second messenger for Ca²⁺ mobilization from the endoplasmatic reticulum.

Several anti-CD38 antibodies are described in the literature, for instance in WO 2006/099875 A1, WO2008037257 A2, WO 2011/154453 A1, WO 2007/042309 A1, WO 2008/047242 A1, WO2012/092612 A1, Cotner, Hemler et al. 1981; Ausiello, Urbani et al. 2000; Lande, Urbani et al. 2002; de Weers, Tai et al. 2011; Deckert, Wetzel et al. 2014; Raab, Goldschmidt et al. 2015; Eissler, Filosto et al. 2018; Roepcke, Plock et al. 2018; and Schooten 2018.

CD38 antibodies may affect CD38 expressing tumor cells by one or more of the following mechanisms of action: complement-dependent cytotoxicity (CDC), antibody-dependent cellular cytotoxicity (ADCC), antibody-dependent cellular phagocytosis (ADCP), programmed cell death, trogocytosis, elimination of immune suppressor cells and modulation of enzymatic activity (van de Donk, Janmaat et al. 2016; Krejcik, Casneuf et al. 2016; Krejcik, Frerichs et al. 2017; Chatterjee, Daenthanasanmak et al. 2018; van de Donk 2018). However, in 2014, it was proposed that, no CD38 antibodies had been described that could induce effective CDC, ADCC, ADCP as well as effectively inhibit CD38 enzyme activity (Lammerts van Bueren, Jakobs et al. 2014).

Optimization of the effector functions may improve the effectivity of therapeutic antibodies for treating cancer or other diseases, e.g., to improve the ability of an antibody to elicit an immune response to antigen-expressing cells. Such efforts are described in, e.g., WO 2013/004842 A2; WO 2014/108198 A1; WO 2018/031258 A1; Dall'Acqua, Cook et al. 2006; Moore, Chen et al. 2010; Desjarlais and Lazar 2011; Kaneko and Niwa 2011; Song, Myojo et al. 2014; Brerski and Georgiou 2016; Sondermann and Szymkowski 2016; Zhang, Armstrong et al. 2017; Wang, Mathieu et al. 2018.

Despite these and other efforts in the art, however, there is a need for CD38 therapeutic antibodies with modulated potencies.

### SUMMARY OF THE INVENTION

The present invention concerns variants of CD38 antibody C, particularly variants having one or more mutations in the Fc region. At least one of these mutations is in a residue corresponding to E430, E345 or S440 in a human IgG1 heavy chain, wherein the amino acid residues are numbered according to the EU index.

So, in one aspect, the invention relates to an antibody variant binding to human CD38, the antibody variant comprising
(a) an antigen-binding region comprising a VH CDR1 having the sequence as set forth in SEQ ID NO:2, a VH CDR2 having the sequence as set forth in SEQ ID NO:3, a VH CDR3 having the sequence as set forth in SEQ ID NO:4, a VL CDR1 having the sequence as set forth in SEQ ID NO:6, a VL CDR2 having the sequence AAS, and a VL CDR3 having the sequence as set forth in SEQ ID NO:7, and
(b) a variant Fc region comprising a mutation in one or more amino acid residues selected from the group corresponding to E430, E345 and S440 in a human IgG1 heavy chain, wherein the amino acid residues are numbered according to the EU index.

In one aspect, the invention relates to an antibody variant binding to human CD38, the antibody variant comprising
(a) a heavy chain comprising a VH region comprising a VH CDR1 having the sequence as set forth in SEQ ID NO:2, a VH CDR2 having the sequence as set forth in SEQ ID NO:3, a VH CDR3 having the sequence as set forth in SEQ ID NO:4 and a human IgG1 CH region with a mutation in one or more of E430, E345 and S440, the amino acid residues being numbered according to the EU index;
(b) a light chain comprising a VL region comprising a VL CDR1 having the sequence as set forth in SEQ ID NO:6, a VL CDR2 having the sequence AAS, and a VL CDR3 having the sequence as set forth in SEQ ID NO:7.

In one aspect, the invention relates to an antibody variant binding to human CD38, the antibody variant comprising
(a) a heavy chain comprising a VH region comprising SEQ ID NO:1 and a human IgG1 CH region with a mutation in one or more of E430, E345 and S440, wherein the amino acid residue numbering is according to the EU index, and
(b) a light chain comprising a VL comprising SEQ ID NO:5.

In one aspect, the invention relates to an isolated nucleic acid encoding the antibody variant according to any aspect or embodiment herein.

In one aspect, the invention relates to an expression vector comprising such a nucleic acid.

In one aspect, the invention relates to a recombinant host cell which produces an antibody variant according to any aspect or embodiment herein.

In one aspect, the invention relates to a method of producing an antibody variant according to any aspect or embodiment herein, comprising cultivating such a recombinant host cell in a culture medium and under conditions suitable for producing the antibody variant.

In one aspect, the invention relates to a method of increasing an effector function of a parent antibody comprising an Fc region and an antigen-binding region binding to CD38, which method comprises introducing into the Fc region a mutation in one or more amino acid residues selected from the group corresponding to E430, E345, and S440 in the Fc region of a human IgG1 heavy chain, wherein the amino acid residues are numbered according to the EU index;
wherein the antigen-binding region comprises a VH CDR1 having the sequence as set forth in SEQ ID NO:2, a VH CDR2 having the sequence as set forth in SEQ ID NO:3, a VH CDR3 having the sequence as set forth in SEQ ID NO:4, a VL CDR1 having the sequence as set forth in SEQ ID NO:6, a VL CDR2 having the sequence AAS, and a VL CDR3 having the sequence as set forth in SEQ ID NO:7.

In some embodiments of the aspects described herein, the mutation in the one or more amino acid residues is selected from the group consisting of E430G, E345K, E430S, E430F, E430T, E345Q, E345R, E345Y, S440Y and S440W, such as, for example, E430G.

In one aspect, the invention relates to a method of producing a variant of a parent antibody comprising an Fc region and an antigen-binding region binding to CD38, the variant having an increased effector function as compared to the parent antibody, which method comprises
(a) introducing into the Fc region a mutation in one or more amino acid residues selected from the group corresponding to E430, E345, and S440 in the Fc region of a human IgG1 heavy chain to obtain a variant antibody,
(b) selecting any variant antibody having an increased effector function as compared to the parent antibody, and
(c) producing said variant antibody in a recombinant host cell,
wherein the antigen-binding region comprises a VH CDR1 having the sequence as set forth in SEQ ID NO:2, a VH CDR2 having the sequence as set forth in SEQ ID NO:3, a VH CDR3 having the sequence as set forth in SEQ ID NO:4, a VL CDR1 having the sequence as set forth in SEQ ID NO:6, a VL CDR2 having the sequence AAS, and a VL CDR3 having the sequence as set forth in SEQ ID NO:7.

In one aspect, the invention relates to an antibody obtained or obtainable by such a method.

In one aspect, the invention relates to a pharmaceutical composition comprising an antibody variant as defined in any aspect or embodiment herein, and a pharmaceutically acceptable carrier.

In one aspect, the invention relates to an antibody variant according to any aspect or embodiment herein for use as a medicament.

In one aspect, the invention relates to an antibody variant according to any aspect or embodiment herein for use in treating a disease involving cells expressing CD38.

In one aspect, the invention relates to an antibody variant according to any aspect or embodiment herein for use in inducing a CDC-response against a tumor comprising cells expressing CD38.

In one aspect, the invention relates to an antibody variant according to any aspect or embodiment herein for use in treating or preventing a cancer in a subject comprising cells expressing human CD38.

In one aspect, the invention relates to an antibody variant according to any aspect or embodiment herein for use in treating or preventing rheumatoid arthritis.

In one aspect, the invention relates to a method for treating a disease comprising cells expressing CD38, comprising administering the antibody variant according to any aspect or embodiment herein to a patient in need thereof, optionally wherein the antibody variant or pharmaceutical composition is administered in a therapeutically effective amount and/or for a time sufficient to treat the disease.

These and other aspect and embodiments of the invention are described in more detail below.

### LEGENDS TO THE FIGURES

FIG. 1 shows an amino acid sequence alignment using Clustal 2.1 software for human IgG1m(a), IgG1m(f), IgG2, IgG3 and IgG4 Fc segments corresponding to residues P247 to K447 in the human IgG1 heavy chains, wherein the amino acid residues are numbered according to the EU index as set forth in Kabat. The amino acid sequences shown correspond to residues 130 to 330 in the heavy chain constant regions of the allotypic variants of human IgG1 designated IgG1m(za) (SEQ ID NO:64; UniProt accession No. P01857), IgG1m(f) (SEQ ID NO:65), IgG1m(z) (SEQ ID NO:66), IgG1m(a) (SEQ ID NO:67) and IgG1m(x) (SEQ ID NO:68); residues 126 to 326 of the IgG2 heavy chain constant region (SEQ ID NO:79; UniProt accession No. P01859); residues 177 to 377 of the IgG3 heavy chain constant region (SEQ ID NO:80; UniProt accession No. P01860), and residues 127 to 327 of the IgG4 heavy chain constant region (SEQ ID NO:81; UniProt accession No. P01861).

FIG. 2 shows the binding of CD38 antibody variants IgG1-A-E430G, IgG1-B-E430G and IgG1-C-E430G to CD38 expressing NALM16 cells in comparison to CD38 antibodies IgG1-A, IgG1-B, IgG1-C and isotype control antibody. For more details, see Example 2.

FIGS. 3A and 3B show the binding of CD38 antibody variants IgG1-A-E430G, IgG1-B-E430G and IgG1-C-E430G to CD38 expressed on cynomolgus PBMCs (FIG. 3A) or Daudi cells expressing high copy numbers of human CD38 (FIG. 3B) in comparison to isotype control antibody. For more details, see Example 2.

FIGS. 4A-4H show the percentage lysis induced by CD38 antibody variants IgG1-A-E430G, IgG1-B-E430G and IgG1-C-E430G of Ramos (FIG. 4A), Daudi (FIG. 4B), Wien-133 (FIG. 4C), NALM-16 (FIG. 4D), REH (FIG. 4E), RS4; 11 (FIG. 4F), U266 (FIG. 4G) and RC-K8 (FIG. 4H) tumor cell lines in a CDC assay as compared to CD38 antibodies IgG1-A, IgG1-B and IgG1-C. For more details, see Example 3.

FIGS. 5A-5C show the effect of CD38 antibody variants IgG1-A-E430G, IgG1-B-E430G and IgG1-C-E430G on the number of viable NK cells (FIG. 5A), T cells (FIG. 5B) and B cells (FIG. 5C) in a CDC assay performed on whole blood as compared to CD38 antibodies IgG1-A, IgG1-B and IgG1-C. For more details, see Example 3.

FIG. 6 shows the percentage lysis of Daudi cells induced by CD38 antibody variants IgG1-A-E430G, IgG1-B-E430G and IgG1-C-E430G in a chromium-release ADCC assay as compared to CD38 antibodies IgG1-A, IgG1-B, IgG1-C and isotype control antibody. For more details, see Example 4.

FIG. 7 shows the dose-dependent FcγRIIIa cross-linking of CD38 antibody variants IgG1-A-E430G, IgG1-B-E430G and IgG1-C-E430G in an ADCC reporter assay as compared to CD38 antibodies IgG1-A, IgG1-B, IgG1-C and isotype control antibody. For more details, see Example 4.

FIGS. 8A and 8B show the effect of CD38 antibody variants IgG1-A-E430G, IgG1-B-E430G and IgG1-C-E430G on the percentage of PKH-29^{pos}, CD14^{pos} and CD19^{neg} macrophages in an ADCP assay as compared CD38 antibodies IgG1-A, IgG1-B, IgG1-C and isotype control antibody. For more details, see Example 5.

FIGS. 9A-9G show the percentage lysis induced by CD38 antibody variants IgG1-A-E430G, IgG1-B-E430G and IgG1-C-E430G of Ramos (FIG. 9A), Daudi (FIG. 9B, FIG. 9C), Wien-133 (FIG. 9D, FIG. 9E) and NALM-16 (FIG. 9F, FIG. 9G) tumor cells lines in an apoptosis assay conducted with (FIG. 9C, FIG. 9E, FIG. 9G) or without (FIG. 9A, FIG. 9B, FIG. 9D, FIG. 9F) Fc-cross-linking antibody, as compared to CD38 antibodies IgG1-A, IgG1-B, IgG1-C and isotype control antibody. For more details, see Example 6.

FIG. 10 illustrates the enzymatic activities of CD38.

FIGS. 11A-11C show the effect of CD38 antibody variants IgG1-A-E430G, IgG1-B-E430G and IgG1-C-E430G on the cyclase activity of HisCD38 (FIG. 11A), Daudi cells (FIG. 11B) and Wien-133 cells (FIG. 11C) as reflected by % NDG conversion over time, in comparison to CD38 antibodies IgG1-A, IgG1-B, IgG1-C and isotype control antibody.

FIGS. 12A-12D show the effect of CD38 antibody variants IgG1-A-E430G, IgG1-B-E430G and IgG1-C-E430G on the CD38 expression on Daudi cells after 45 minute co-culture with macrophages in comparison to CD38 antibodies IgG1-A, IgG1-B, IgG1-C and isotype control antibody. Macrophages were from Donor A (FIG. 12A, FIG. 12B) or Donor B (FIG. 12B, FIG. 12D) and antibody opsonized cells were tested for CD38 expression (FIG. 12A, FIG. 12B) or human IgG staining (FIG. 12C, FIG. 12D).

FIG. 13 shows the effect of CD38 antibody variants IgG1-B-E430G and IgG1-C-E430G on the CD38 expression on T regulatory cells with or without PBMCs, in comparison to IgG1-B.

FIG. 14 shows the percentage lysis induced by CD38 antibody variants IgG1-A-E430G (closed triangles), IgG1-B-E430G (closed circles) and IgG1-C-E430G (closed squares) of different B cell tumor cell lines in a CDC assay as compared to CD38 antibodies IgG1-B (open circle) and isotype control antibody (open diamonds). For more details, see Example 3.

FIG. 15 shows a summary of some of the EC50 values depicted in Table 4. EC50 values of CDC induced by antibodies IgG1-B, IgG1-B-E430G and IgG1-C-E430G on 20 different B cell tumor cell lines are shown. Each square, triangle or circle represents a different B cell tumor cell line. EC50 values obtained with AML cell lines were not included because IgG1-B-E430G was not tested on AML cell lines.

FIG. 16 shows the percentage lysis induced by CD38 antibody variant IgG1-C-E430G (closed circles) of different AML tumor cell lines in a CDC assay as compared to CD38 antibodies IgG1-B (open circles) and isotype control antibody (closed squares). For more details, see Example 3.

FIG. 17 shows the percentage lysis induced by CD38 antibody variants IgG1-B-E430G (closed circles) and IgG1-C-E430G (closed squares) of T regulatory cells in a CDC assay as compared to CD38 antibodies IgG1-B (open circles). For more details, see Example 3.

FIG. 18 shows the percentage lysis of Daudi, Wien-133, Granta 519 and MEC-2 cells induced by CD38 antibody variants IgG1-B-E430G, IgG1-C-E430G in a chromium-release ADCC assay as compared to CD38 antibodies IgG-B, IgG1-C and IgG1-b12-E430G. For more details, see Example 4.

FIG. 19 shows the dose-dependent FcγRIIIa cross-linking of CD38 antibody variants IgG1-A-E430G, IgG1-B-E430G and IgG1-C-E430G in an ADCC reporter assay with T regulatory cells as compared to CD38 antibodies IgG1-A, IgG1-B, IgG1-C and isotype control antibody. For more details, see Example 4.

FIG. 20 shows the tumor size (mm³) in mice treated with either CD38 antibody variant IgG1-C-E430G or PBS (negative control). For more details see Example 9.

FIG. 21 illustrates the assay setup to measure trogocytosis. 1) Daudi cells were labelled with PKH-26 (membrane staining) and cell trace violet (cytosol staining) and opsonized with CD38 antibodies. 2) Labelled Daudi cells and macrophages were co-incubated for 2 h at 37° C. to allow macrophage attachment. 3) Cell membrane transfer or trogocytosis from Daudi cells to macrophages. 4) Detachment of the macrophage-Daudi interaction and degradation of the Daudi cell membrane in the macrophage. For more details see Example 8.

FIGS. 22A-22D show complement-mediated cytotoxicity by IgG1-C-E430G or Darzalex® in bone marrow mononuclear cells from 3 newly diagnosed MM patients (FIG. 22A, FIG. 22B and FIG. 22D) and 1 relapsed/refractory MM patient (FIG. 22C).

### DETAILED DESCRIPTION OF THE INVENTION

In describing the embodiments of the invention specific terminology will be resorted to for the sake of clarity. However, the invention is not intended to be limited to the specific terms so selected, and it is understood that each specific term includes all technical equivalents which operate in a similar manner to accomplish a similar purpose.

### Definitions

As used herein, the term “CD38” generally refers to human CD38 (UniProtKB—P28907 (CD38_HUMAN)) having the sequence set forth in SEQ ID NO:38, but may also, unless contradicted by context, refer to variants, isoforms and orthologs thereof. Variants of human CD38 with S274, Q272R, T237A or D202G mutations are described in WO 2006/099875 A1 and WO 2011/154453 A1.

The term “immunoglobulin” refers to a class of structurally related glycoproteins consisting of two pairs of polypeptide chains, one pair of light (L) low molecular weight chains and one pair of heavy (H) chains, all four potentially inter-connected by disulfide bonds. The structure of immunoglobulins has been well characterized. See for instance Fundamental Immunology Ch. 7 (Paul, W., ed., 2nd ed. Raven Press, N.Y. (1989)). Briefly, each heavy chain typically is comprised of a heavy chain variable (VH) region and a heavy chain constant (CH) region. The CH region typically is comprised of three domains, CH1, CH2, and CH3. The heavy chains are typically inter-connected via disulfide bonds in the so-called “hinge region”. Each light chain typically is comprised of a light chain variable (VL) region and a light chain constant region, the latter typically comprised of one domain, CL. The VH and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each VH and VL region is typically composed of three CDRs and four FRs, arranged from amino-terminus to carboxy-terminus in the following order: FR1, CDR1, FR2, CDR2, FR3, CDR3, FR4 (see also Chothia and Lesk J. Mol. Biol. 196, 901 917 (1987)).

Unless otherwise stated or contradicted by context, CDR sequences herein are identified according to IMGT rules using DomainGapAlign (Lefranc M P., Nucleic Acids Research 1999; 27:209-212 and Ehrenmann F., Kaas Q. and Lefranc M.-P. Nucleic Acids Res., 38, D301-307 (2010); see also internet http address www.imgt.org/.

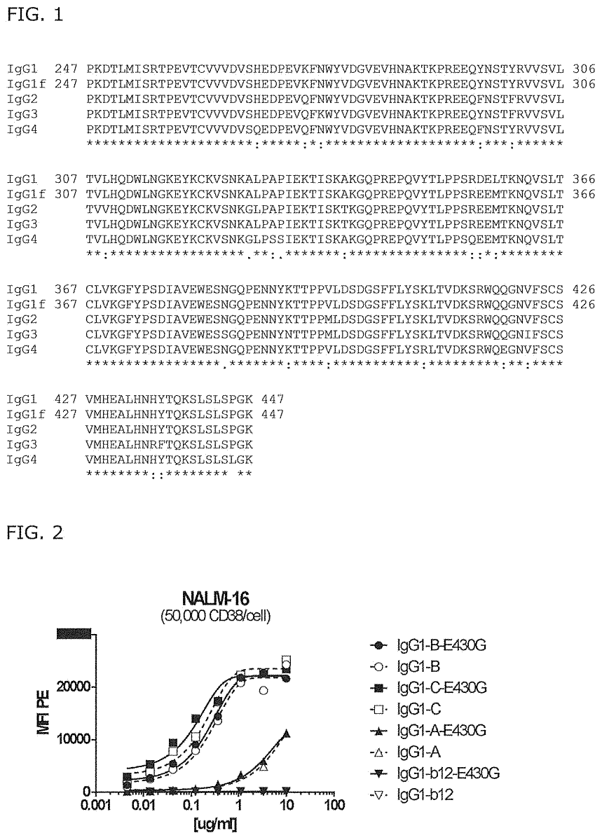

Unless otherwise stated or contradicted by context, reference to amino acid positions in the CH or Fc region/Fc domain in the present invention is according to the EU-numbering (Edelman et al., Proc Natl Acad Sci USA. 1969 May; 63(1):78-85; Kabat et al., Sequences of proteins of immunological interest. 5th Edition—1991 NIH Publication No. 91-3242). An amino acid residue in a CH of another isotype than human IgG1 may, however, alternatively be referred to by the corresponding amino acid position in a wild-type human IgG1 heavy chain in which the amino acid residues are numbered according to the EU index. Specifically, the corresponding amino acid position can be identified as illustrated in FIG. 1, i.e., by (a) aligning the amino acid sequence of the non-IgG1 constant region (or a segment thereof) with the amino acid sequence of a human IgG1 heavy chain (or segment thereof) in which the amino acid residues are numbered according to the EU index, and (b) identifying which amino acid position in the IgG1 heavy chain the amino acid residue is aligned with. Accordingly, the position of such an amino acid residue can herein be referred to as “the amino acid residue at a position corresponding to”, followed by the amino acid position in a wild-type human IgG1 heavy chain numbered according to the EU index. When referring to one or more of a number of different amino acid positions, this can be referred to herein as “a mutation in one or more amino acid residues at positions selected from the group consisting of the positions corresponding to”, “a mutation in one or more amino acid residues at positions corresponding to” or simply “a mutation in one or more amino acid residues selected from the group corresponding to”, followed by two or more amino acid positions (e.g., E430, E345 and S440) in a human wild-type IgG1 heavy chain, wherein the amino acid residues are numbered according to the EU index.

The term “hinge region” as used herein is intended to refer to the hinge region of an immunoglobulin heavy chain. Thus, for example the hinge region of a human IgG1 antibody corresponds to amino acids 216-230 according to the EU numbering.

The term “CH2 region” or “CH2 domain” as used herein is intended to refer to the CH2 region of an immunoglobulin heavy chain. Thus, for example the CH2 region of a human IgG1 antibody corresponds to amino acids 231-340 according to the EU numbering. However, the CH2 region may also be any of the other subtypes as described herein.

The term “CH3 region” or “CH3 domain” as used herein is intended to refer to the CH3 region of an immunoglobulin heavy chain. Thus, for example the CH3 region of a human IgG1 antibody corresponds to amino acids 341-447 according to the EU numbering. However, the CH3 region may also be any of the other subtypes as described herein.

The term “antibody” (Ab) in the context of the present invention refers to an immunoglobulin molecule, a fragment of an immunoglobulin molecule, or a derivative of either thereof, which has the ability to specifically bind to an antigen. The antibody of the present invention comprises an Fc-domain of an immunoglobulin and an antigen-binding region. An antibody generally contains two CH2-CH3 regions and a connecting region, e.g. a hinge region, e.g. at least an Fc-domain. Thus, the antibody of the present invention may comprise an Fc region and an antigen-binding region. The variable regions of the heavy and light chains of the immunoglobulin molecule contain a binding domain that interacts with an antigen. The constant or “Fc” regions of the antibodies may mediate the binding of the immunoglobulin to host tissues or factors, including various cells of the immune system (such as effector cells) and components of the complement system such as C1q, the first component in the classical pathway of complement activation. As used herein, unless contradicted by context, the Fc region of an immunoglobulin typically contains at least a CH2 domain and a CH3 domain of an immunoglobulin CH, and may comprise a connecting region, e.g., a hinge region. An Fc-region is typically in dimerized form via, e.g., disulfide bridges connecting the two hinge regions and/or non-covalent interactions between the two CH3 regions. The dimer may be a homodimer (where the two Fc region monomer amino acid sequences are identical) or a heterodimer (where the two Fc region monomer amino acid sequences differ in one or more amino acids). Preferably, the dimer is a homodimer. An Fc region-fragment of a full-length antibody can, for example, be generated by digestion of the full-length antibody with papain, as is well-known in the art. An antibody as defined herein may, in addition to an Fc region and an antigen-binding region, further comprise one or both of an immunoglobulin CH1 region and a CL region. An antibody may also be a multispecific antibody, such as a bispecific antibody or similar molecule. The term “bispecific antibody” refers to an antibody having specificities for at least two different, typically non-overlapping, epitopes. Such epitopes may be on the same or different targets. If the epitopes are on different targets, such targets may be on the same cell or different cells or cell types. As indicated above, unless otherwise stated or clearly contradicted by the context, the term antibody herein includes fragments of an antibody which comprise at least a portion of an Fc-region and which retain the ability to specifically bind to the antigen. Such fragments may be provided by any known technique, such as enzymatic cleavage, peptide synthesis and recombinant expression techniques. It has been shown that the antigen-binding function of an antibody may be performed by fragments of a full-length antibody. Examples of binding fragments encompassed within the term “Ab” or “antibody” include, without limitation, monovalent antibodies (described in WO2007059782 by Genmab); heavy-chain antibodies, consisting only of two heavy chains and naturally occurring in e.g. camelids (e.g., Hamers-Casterman (1993) Nature 363:446); ThioMabs (Roche, WO2011069104), strand-exchange engineered domain (SEED or Seed-body) which are asymmetric and bispecific antibody-like molecules (Merck, WO2007110205); Triomab (Pharma/Fresenius Biotech, Lindhofer et al. 1995 J Immunol 155:219; WO2002020039); FcLAdp (Regeneron, WO2010151792), Azymetric Scaffold (Zymeworks/Merck, WO2012/058768), mAb-Fv (Xencor, WO2011/028952), Xmab (Xencor), Dual variable domain immunoglobulin (Abbott, DVD-Ig, U.S. Pat. No. 7,612,181); Dual domain double head antibodies (Unilever; Sanofi Aventis, WO20100226923), Di-diabody (ImClone/Eli Lilly), Knobs-into-holes antibody formats (Genentech, WO9850431); DuoBody (Genmab, WO 2011/131746); Bispecific IgG1 and IgG2 (Pfizer/Rinat, WO11143545), DuetMab (MedImmune, US2014/0348839), Electrostatic steering antibody formats (Amgen, EP1870459 and WO 2009089004; Chugai, US201000155133; Oncomed, WO2010129304A2); bispecific IgG1 and IgG2 (Rinat neurosciences Corporation, WO11143545), CrossMAbs (Roche, WO2011117329), LUZ-Y (Genentech), Biclonic (Merus, WO2013157953), Dual Targeting domain antibodies (GSK/Domantis), Two-in-one Antibodies or Dual action Fabs recognizing two targets (Genentech, NovImmune, Adimab), Cross-linked Mabs (Karmanos Cancer Center), covalently fused mAbs (AIMM), CovX-body (CovX/Pfizer), FynomAbs (Covagen/Janssen ilag), DutaMab (Dutalys/Roche), iMab (MedImmune), IgG-like Bispecific (ImClone/Eli Lilly, Shen, J., et al. J Immunol Methods, 2007. 318(1-2): p. 65-74), TIG-body, DIG-body and PIG-body (Pharmabcine), Dual-affinity retargeting molecules (Fc-DART or Ig-DART, by Macrogenics, WO/2008/157379, WO/2010/080538), BEAT (Glenmark), Zybodies (Zyngenia), approaches with common light chain (Crucell/Merus, U.S. Pat. No. 7,262,028) or common heavy chains (κλBodies by NovImmune, WO2012023053), as well as fusion proteins comprising a polypeptide sequence fused to an antibody fragment containing an Fc-region like scFv-fusions, like BsAb by ZymoGenetics/BMS, HERCULES by Biogen Idec (U.S. Ser. No. 00/795,1918), SCORPIONS by Emergent BioSolutions/Trubion and Zymogenetics/BMS, Ts2Ab (MedImmune/AZ (Dimasi, N., et al. J Mol Biol, 2009. 393(3): p. 672-92), scFv fusion by Genentech/Roche, scFv fusion by Novartis, scFv fusion by Immunomedics, scFv fusion by Changzhou Adam Biotech Inc (CN 102250246), TvAb by Roche (WO 2012025525, WO 2012025530), mAb² by f-Star (WO2008/003116), and dual scFv-fusion s. It should be understood that the term antibody, unless otherwise specified, includes monoclonal antibodies (such as human monoclonal antibodies), polyclonal antibodies, chimeric antibodies, humanized antibodies, monospecific antibodies (such as bivalent monospecific antibodies), bispecific antibodies, antibodies of any isotype and/or allotype; antibody mixtures (recombinant polyclonals) for instance generated by technologies exploited by Symphogen and Merus (Oligoclonics), multimeric Fc proteins as described in WO2015/158867, and fusion proteins as described in WO2014/031646. While these different antibody fragments and formats are generally included within the meaning of antibody, they collectively and each independently are unique features of the present invention, exhibiting different biological properties and utility.

A “CD38 antibody” or “anti-CD38 antibody” as described herein is an antibody which binds specifically to the antigen CD38.

The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations, insertions or deletions introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The terms “monoclonal antibody”, “monoclonal Ab”, “monoclonal antibody composition”, “mAb”, or the like, as used herein refer to a preparation of Ab molecules of single molecular composition. A monoclonal antibody composition displays a single binding specificity and affinity for a particular epitope. Accordingly, the term “human monoclonal antibody” refers to Abs displaying a single binding specificity which have variable and constant regions derived from human germline immunoglobulin sequences. The human mAbs may be generated by a hybridoma which includes a B cell obtained from a transgenic or trans-chromosomal non-human animal, such as a transgenic mouse, having a genome comprising a human heavy chain transgene repertoire and a light chain transgene repertoire, rearranged to produce a functional human antibody and fused to an immortalized cell.

As used herein, “isotype” refers to the immunoglobulin class that is encoded by heavy chain constant region genes, including, for instance, IgG1, IgG2, IgG3, IgG4, IgD, IgA1, IgA2, IgE, and IgM, as well as any allotypes thereof such as IgG1m(z), IgG1m(a), IgG1m(x), IgG1m(f) and mixed allotypes thereof such as IgG1m(za), IgG1m(zax), IgG1m(fa), etc. (see, for instance, de Lange, Experimental and Clinical Immunogenetics 1989; 6(1):7-17).

Further, each heavy chain isotype can be combined with either a kappa (κ) or lambda (λ) light chain. The term “mixed isotype” used herein refers to Fc region of an immunoglobulin generated by combining structural features of one isotype with the analogous region from another isotype thereby generating a hybrid isotype. A mixed isotype may comprise an Fc region having a sequence comprised of two or more isotypes selected from the following IgG1, IgG2, IgG3, IgG4, IgD, IgA1, IgGA2, IgE, or IgM thereby generating combinations such as e.g. IgG1/IgG3, IgG1/IgG4, IgG2/IgG3, IgG2/IgG4 or IgG1/IgA.

The term “full-length antibody” when used herein, refers to an antibody (e.g., a parent or variant antibody) which contains all heavy and light chain constant and variable domains corresponding to those that are normally found in a wild-type antibody of the isotype in question.

A “full-length bivalent, monospecific monoclonal antibody” when used herein, refers to a bivalent, monospecific antibody (e.g., a parent or variant antibody) formed by a pair of identical HCs and a pair of identical LCs, with the constant and variable domains corresponding to those normally found in an antibody of the particular isotype in question.

The term “antigen-binding region”, “antigen binding region”, “binding region” or antigen binding domain, as used herein, refers to a region of an antibody which is capable of binding to the antigen. This binding region is typically defined by the VH and VL domains of the antibody which may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). The antigen can be any molecule, such as a polypeptide, e.g. present on a cell.

The term “target”, as used herein, refers to a molecule to which the antigen binding region of the antibody binds. The target includes any antigen towards which the raised antibody is directed. The term “antigen” and “target” may in relation to an antibody be used interchangeably and constitute the same meaning and purpose with respect to any aspect or embodiment of the present invention.

The term “epitope” means a protein determinant capable of specific binding to an antibody variable domain. Epitopes usually consist of surface groupings of molecules such as amino acids, sugar side chains or a combination thereof and usually have specific three-dimensional structural characteristics, as well as specific charge characteristics. Conformational and non-conformational epitopes are distinguished in that the binding to the former but not the latter is lost in the presence of denaturing solvents. The epitope may comprise amino acid residues directly involved in the binding (also called immunodominant component of the epitope) and other amino acid residues, which are not directly involved in the binding.

A “variant” as used herein refers to a protein or polypeptide sequence which differs in one or more amino acid residues from a parent or reference sequence. A variant may, for example, have a sequence identity of at least 80%, such as 90%, or 95%, or 97%, or 98%, or 99%, to a parent or reference sequence. Also or alternatively, a variant may differ from the parent or reference sequence by 12 or less, such as 11, 10, 9, 8, 7, 6, 5, 4, 3, 2 or 1 mutation(s) such as substitutions, insertions or deletions of amino acid residues. Accordingly, a “variant antibody” or an “antibody variant”, used interchangeably herein, refers to an antibody that differs in one or more amino acid residues as compared to a parent or reference antibody, e.g., in the antigen-binding region, Fc-region or both. Likewise, a “variant Fc region” or “Fc region variant” refers to an Fc region that differs in one or more amino acid residues as compared to a parent or reference Fc region, optionally differing from the parent or reference Fc region amino acid sequence by 12 or less, such as 11, 10, 9, 8, 7, 6, 5, 4, 3, 2 or 1 mutation(s) such as substitutions, insertions or deletions of amino acid residues. The parent or reference Fc region is typically the Fc region of a human wild-type antibody which, depending on the context, may be a particular isotype. A variant Fc region may, in dimerized form, be a homodimer or heterodimer, e.g., where one of the amino acid sequences of the dimerized Fc region comprises a mutation while the other is identical to a parent or reference wild-type amino acid sequence. Examples of wild-type (typically a parent or reference sequence) IgG CH and variant IgG constant region amino acid sequences, which comprise Fc region amino acid sequences, are set out in Table 1.

In the context of the present invention, conservative substitutions may be defined as substitutions within the following classes of amino acids:
Acidic Residues: Asp (D) and Glu (E)
Basic Residues: Lys (K), Arg (R), and His (H)
Hydrophilic Uncharged Residues: Ser (S), Thr (T), Asn (N), and Gln (Q)
Aliphatic Uncharged Residues: Gly (G), Ala (A), Val (V), Leu (L), and Ile (I)
Non-polar Uncharged Residues: Cys (C), Met (M), and Pro (P)
Aromatic Residues: Phe (F), Tyr (Y), and Trp (W)

Alternative conservative amino acid residue substitution classes:
1. A S T
2. D E
3. N Q
4. R K
5. I L M
6. F Y W

Alternative Physical and Functional Classifications of Amino Acid Residues:
Alcohol group-containing residues: S and T
Aliphatic residues: I, L, V, and M
Cycloalkenyl-associated residues: F, H, W, and Y
Hydrophobic residues: A, C, F, G, H, I, L, M, R, T, V, W, and Y
Negatively charged residues: D and E
Polar residues: C, D, E, H, K, N, Q, R, S, and T
Positively charged residues: H, K, and R
Small residues: A, C, D, G, N, P, S, T, and V
Very small residues: A, G, and S
Residues involved in turn formation: A, C, D, E, G, H, K, N, Q, R, S, P, and T
Flexible residues: Q, T, K, S, G, N, D, E, and R

“Sequence identity” as used herein refers to the percent identity between two sequences as a function of the number of identical positions shared by the sequences (i.e., percent homology=# of identical positions/total # of positions×100), taking into account the number of gaps, and the length of each gap, which need to be introduced for optimal alignment of the two sequences. The percent identity between two nucleotide or amino acid sequences may e.g. be determined using the algorithm of E. Meyers and W. Miller, Comput. Appl. Biosci 4, 11-17 (1988) that has been incorporated into the ALIGN program (version 2.0), using a PAM 120 weight residue table, a gap length penalty of 12 and a gap penalty of 4. In addition, the percent identity between two amino acid sequences may be determined using the Needleman and Wunsch, J. Mol. Biol. 48, 444-453 (1970) algorithm. Other tools for sequence alignments are publicly available on the internet, and include, without limitation, Clustal Omega and EMBOSS Needle on the EMBL-EBI website www.ebi.ac.uk. Typically, default settings can be used.

In the context of the present invention the following notations are, unless otherwise indicated, used to describe a mutation; name of amino acid which is mutated, followed by the position number which is mutated, followed by what the mutation encompasses. Thus if the mutation is a substitution, the name of the amino acid which replaces the prior amino acid is included, if the amino acid is deleted it is indicated by a “*”, if the mutation is an addition the amino acid being added is included after the original amino acid. Amino acid names may be one or three-letter codes. Thus for example; the substitution of a glutamic acid in position 430 with a glycine is referred to as E430G, substitution of glutamic acid in position 430 with any amino acid is referred to as E430X, deletion of glutamic acid in position 430 is referred to as E430* and addition of a proline after glutamic acid at position E430 is referred to as E430EP.

As used herein, “immunosuppressive cells” refer to immune cells which may suppress an immune response in a subject, such as by suppressing the activity of effector T cells and/or inhibiting T cell proliferation. Examples of such immunosuppressive cells include, but are not limited to, regulatory T cells (Tregs), regulatory B cells (Bregs) and myeloid-derived suppressor cells (MDSCs). There are also immunosuppressive NK cells, NKT cells, macrophages and antigen-presenting cells (APCs). An example of a phenotype for an immunosuppressive NK cell is CD56^{bright}CD16⁻.

“Regulatory T cells” or “Tregs” or “Treg” refers to T lymphocytes that regulate the activity of other T cell(s) and/or other immune cells, usually by suppressing their activity. An example of a Treg phenotype is CD3⁺CD4⁺CD25⁺CD127^{dim}. Tregs may further express Foxp3. It is appreciated that Tregs may not be fully restricted to this phenotype.

“Effector T cells” or “Teffs” or “Teff” refers to T lymphocytes that carry out a function of an immune response, such as killing tumor cells and/or activating an antitumor immune-response which can result in clearance of the tumor cells from the body. Examples of Teff phenotypes include CD3⁺CD4⁺ and CD3⁺CD8⁺. Teffs may secrete, contain or express markers such as IFNγ, granzyme B and ICOS. It is appreciated that Teffs may not be fully restricted to these phenotypes.

“Myeloid-derived suppressor cells” or “MDSCs” or “MDSC” refers to a specific population of cells of the hematopoietic lineage that express the macrophage/monocyte marker CD11b and the granulocyte marker Gr-1/Ly-6G. An example of an MDSC phenotype is CD11b⁺HLA-DR⁻CD14⁻CD33⁺CD15⁺. MDSCs typically also show low or undetectable expression of the mature antigen presenting cell markers MHC Class II and F480. MDSCs are immature cells of the myeloid lineage and may further differentiate into other cell types, such as macrophages, neutrophils, dendritic cells, monocytes or granulocytes. MDSCs may be found naturally in normal adult bone marrow of human and animals or in sites of normal hematopoiesis, such as the spleen.

“Regulatory B cell” or “Breg” or “Bregs” refers to B lymphocytes that suppress immune responses. An example of a Breg phenotype is CD19⁺CD24⁺CD38⁺. Bregs may suppress immune responses by inhibiting T cell proliferation mediated by IL-10 secreted by the Bregs. It is appreciated that other Breg subsets exists, and are described in for example Ding et al., (2015) Human Immunology 76: 615-621.

As used herein, the term “effector cell” refers to an immune cell which is involved in the effector phase of an immune response. Exemplary immune cells include a cell of a myeloid or lymphoid origin, for instance lymphocytes (such as B cells and T cells including cytolytic T cells (CTLs)), killer cells, natural killer cells, macrophages, monocytes, eosinophils, polymorphonuclear cells, such as neutrophils, granulocytes, mast cells, and basophils. Some effector cells express Fc receptors (FcRs) or complement receptors and carry out specific immune functions. In some embodiments, an effector cell such as, e.g., a natural killer cell, is capable of inducing ADCC. For example, monocytes, macrophages, neutrophils, dendritic cells and Kupffer cells which express FcRs, are involved in specific killing of target cells and/or presenting antigens to other components of the immune system, or binding to cells that present antigens. In some embodiments the ADCC can be further enhanced by antibody driven classical complement activation resulting in the deposition of activated C3 fragments on the target cell. C3 cleavage products are ligands for complement receptors (CRs), such as CR3, expressed on myeloid cells. The recognition of complement fragments by CRs on effector cells may promote enhanced Fc receptor-mediated ADCC. In some embodiments antibody driven classical complement activation leads to C3 fragments on the target cell. These C3 cleavage products may promote direct complement-dependent cellular cytotoxicity (CDCC). In some embodiments, an effector cell may phagocytose a target antigen, target particle or target cell which may depend on antibody binding and mediated by FcγRs expressed by the effector cells. The expression of a particular FcR or complement receptor on an effector cell may be regulated by humoral factors such as cytokines. For example, expression of FcγRI has been found to be up-regulated by interferon γ (IFN γ) and/or G-CSF. This enhanced expression increases the cytotoxic activity of FcγRI-bearing cells against targets. An effector cell can phagocytose a target antigen or phagocytose or lyse a target cell. In some embodiments antibody driven classical complement activation leads to C3 fragments on the target cell. These C3 cleavage products may promote direct phagocytosis by effector cells or indirectly by enhancing antibody mediated phagocytosis.

The term “Fc effector functions,” as used herein, is intended to refer to functions that are a consequence of binding a polypeptide or antibody to its target, such as an antigen, on a cell membrane wherein the Fc effector function is attributable to the Fc region of the polypeptide or antibody. Examples of Fc effector functions include (i) C1q-binding, (ii) complement activation, (iii) complement-dependent cytotoxicity (CDC), (iv) antibody-dependent cell-mediated cytotoxity (ADCC), (v) Fc-gamma receptor-binding, (vi) antibody-dependent cellular phagocytosis (ADCP), (vii) complement-dependent cellular cytotoxicity (CDCC), (viii) complement-enhanced cytotoxicity, (ix) binding to complement receptor of an opsonized antibody mediated by the antibody, (x) opsonisation, (xi) trogocytosis, and (xii) a combination of any of (i) to (xi).

As used herein, the term “complement activation” refers to the activation of the classical complement pathway, which is initiated by a large macromolecular complex called C1 binding to antibody-antigen complexes on a surface. C1 is a complex, which consists of 6 recognition proteins C1q and a hetero-tetramer of serine proteases, C1r2C1s2. C1 is the first protein complex in the early events of the classical complement cascade that involves a series of cleavage reactions that starts with the cleavage of C4 into C4a and C4b and C2 into C2a and C2b. C4b is deposited and forms together with C2a an enzymatic active convertase called C3 convertase, which cleaves complement component C3 into C3b and C3a, which forms a C5 convertase This C5 convertase splits C5 in C5a and C5b and the last component is deposited on the membrane and that in turn triggers the late events of complement activation in which terminal complement components C5b, C6, C7, C8 and C9 assemble into the membrane attack complex (MAC). The complement cascade results in the creation of pores in the cell membrane which causes lysis of the cell, also known as complement-dependent cytotoxicity (CDC). Complement activation can be evaluated by using C1q efficacy, CDC kinetics CDC assays (as described in WO2013/004842, WO2014/108198) or by the method Cellular deposition of C3b and C4b described in Beurskens et al., J Immunol Apr. 1, 2012 vol. 188 no. 7, 3532-3541.

The term “complement-dependent cytotoxicity” (CDC), as used herein, is intended to refer to the process of antibody-mediated complement activation leading to lysis of the cell to which the antibody is bound, which, without being bound by theory is believed to be the result of pores in the membrane that are created by the assembly of the so-called membrane attack complex (MAC). Suitable assays for evaluating CDC are known in the art and include, for example, in vitro assays in which normal human serum is used as a complement source, as described in Example 3. A non-limiting example of an assay for determining the maximum lysis of CD38 expressing cells as mediated by a CD38 antibody, or the EC50 value, may comprise the steps of:
(a) plating about 100,000 CD38-expressing cells in 40 μL culture medium supplemented with 0.2% BSA per well in a multi-well plate;
(b) preincubating cells for 20 minutes with 40 μL of serially diluted CD38 antibody (0.0002-10 μg/mL);
(c) incubating each well for 45 minutes at 37° C. with 20 percent of pooled normal human serum;
(d) adding a viability dye and measuring the percentage of cell lysis on a flow cytometer;
(e) determining the maximum lysis and/or calculating the EC50 value using non-linear regression.

The term “antibody-dependent cell-mediated cytotoxicity” (“ADCC”) as used herein, is intended to refer to a mechanism of killing of antibody-coated target cells by cells expressing

Fc receptors that recognize the constant region of the bound antibody. Suitable assays for evaluating ADCC are known in the art and include, for example, the assays described in Example 4. Non-limiting examples of assays for determining the ADCC of CD38-expressing cells as mediated by a CD38 antibody may comprise the steps of the ⁵¹Cr-release assay or the reporter assay set out below.

ADCC with ⁵¹Cr Release Assay
(a) plating about 5,000 ⁵¹Cr labelled CD38-expressing cells (e.g., Daudi cells) in 50 μL culture medium supplemented with 0.2% BSA per well in a multi-well plate;
(b) preincubating cells for 15 minutes with 50 μL of serially diluted CD38 antibody (0.0002-10 μg/mL);
(c) incubating each well for 4 hours at 37° C. with 500,000 freshly isolated peripheral blood mononuclear cells (PBMCs) per well;
(d) measuring the amount of ⁵¹Cr release in 75 μL supernatant on a gamma counter;
(e) calculating the percentage of cell lysis as (cpm sample−cpm spontaneous lysis)/(cpm maximal lysis−cpm spontaneous lysis) wherein cpm is counts per minute.
   ADCC with reporter assay
(a) plating about 5,000 CD38-expressing cells (e.g., Daudi cells) in 10 μL in multi-well plates suitable for optical readings (e.g., 384-well OptiPlates from PerkinElmer Inc.) in a standard medium (e.g., RPMI 1640) supplemented with 25% low IgG serum;
(b) incubating each well for 6 hours at 37° C. with 10 μL engineered Jurkat cells stably expressing the FcγRIIIa receptor, V158 (high affinity) variant, and an NFAT response element driving expression of firefly luciferase as effector cells and 10 μL serially diluted CD38 antibody (0.0002-10 μg/mL);
(c) incubating each well 5 minutes at RT with 30 μL Luciferase substrate and measuring luminescence.

The term “antibody-dependent cellular phagocytosis” (“ADCP”) as used herein is intended to refer to a mechanism of elimination of antibody-coated target cells by internalization by phagocytes. The internalized antibody-coated target cells are contained in a vesicle called a phagosome, which then fuses with one or more lysosomes to form a phagolysosome. Suitable assays for evaluating ADCP are known in the art and include, for example, the in vitro cytotoxicity assay with macrophages as effector cells and video microscopy as described by van Bij et al. in Journal of Hepatology Volume 53, Issue 4, October 2010, Pages 677-685, and the in vitro cytotoxicity assay described in Example 5. A non-limiting example of an assay for determining the ADCP of CD38 expressing cells as mediated by a CD38 antibody may comprise the steps of:
(a) differentiating freshly isolated monocytes to macrophages with 5 days incubation in GM-CSF-containing medium;
(b) plating about 100,000 macrophages per well in a multi-well plate in dendritic cell medium with GM-CSF;
(c) adding 20,000 CD38-antibody opsonized CD38-expressing cells (e.g., Daudi cells), labelled with a generic fluorescent membrane dye, per well for 45 minutes at 37° C.;
(d) measuring the percentage of CD14-positive, CD19-negative, membrane-dye-positive macrophages on a flow cytometer.

As used herein, “trogocytosis” refers to a process characterized by the transfer of cell surface molecules from a donor cell to an acceptor cell, such as an effector cell. Typical acceptor cells include T and B cells, monocytes/macrophages, dendritic cells, neutrophils, and NK cells. Trogocytosis-mediated transfer of a cell surface molecule such as, e.g., CD38, from a donor cell to an acceptor cell may also result in the transfer of an antibody-antigen complex from the donor cell to an acceptor cell, i.e., an antibody-antigen complex where an antibody is bound to the cell surface molecule. In particular, a specialized form of trogocytosis may occur when the acceptor cells are Fc-gamma-receptor (FcγR) expressing effector cells; these acceptor cells may take up and internalize donor cell-associated immune complexes composed of specific antibodies bound to target antigens on donor cells, typically after binding of FcγRs to the Fc regions of the antibodies. Suitable assays for evaluating trogocytosis are known in the art and include, for example, the assay in Example 8. Non-limiting examples of assays for determining trogocytosis of CD38 expressing cells as mediated by a CD38 antibody include the following:

### Trogocytosis (Daudi Cells):

(a′) differentiating freshly isolated monocytes to macrophage with 5 days GM-CSF;
(b′) plating about 100,000 macrophages per well in dendritic cell medium with GM-CSF;
(c′) adding about 20,000 CD38 antibody-opsonized Daudi cells, labelled with a generic fluorescent membrane dye, per well for 45 minutes at 37° C.;
(d′) measuring CD38 expression on Daudi cells on a flow cytometer, wherein a reduction in CD38 on CD38-antibody opsonized Daudi cells as compared to a control indicates trogocytosis.

### Trogocytosis (Tregs):

(a) plating about 500,000 freshly isolated PBMCs per well in cell culture medium 0/N at 37° C.;
(b) adding about 100,000, CD38 antibody-opsonized Tregs, labelled with a generic fluorescent intracellular amine dye, per well overnight (0/N) at 37° C.; and
(c) measuring CD38 expression on Tregs on a flow cytometer, wherein a reduction in CD38 on CD38-antibody opsonized Tregs as compared to a control indicates trogocytosis.

The control can be selected by the skilled person based on the specific purpose of the study or assay in question. However, non-limiting examples of controls include (i) the absence of any antibody and (ii) an isotype control antibody. One example of an isotype control antibody is antibody b12, having the VH and VL sequences described in Table 1. In some embodiments where it is desired to evaluate the trogocytosis-effect of an antibody variant as described herein, the control may be (iii) a parent or reference antibody having a different antigen-binding region and/or a different Fc region.

In some embodiments, in step (b), in addition or alternative to the fluorescent intracellular amine dye, the Tregs are labelled with a generic fluorescent membrane dye.

In some embodiments, in step (d′) and (c) of the trogocytosis assays outlined above, the reduction in CD38 antibody on the donor cells can also be measured. For example, in cases where the CD38 antibody is a human IgG (huIgG) antibody, a secondary antibody can be used to detect huIgG.

In addition to Daudi cells (ATCC CCL-213), tumor cells suitable for the first assay include, without limitation, those listed in Table 2, particularly those with a high CD38 expression.

In addition to Tregs, suitable CD38-expressing cells for the second assay include immune cells such as, e.g., NK cells, B cells, T cells and monocytes, as well as tumor cells listed in Table 2, particularly those with a low CD38 expression level.

The term “vector,” as used herein, is intended to refer to a nucleic acid molecule capable of inducing transcription of a nucleic acid segment ligated into the vector. One type of vector is a “plasmid”, which is in the form of a circular double stranded DNA loop. Another type of vector is a viral vector, wherein the nucleic acid segment may be ligated into the viral genome. Certain vectors are capable of autonomous replication in a host cell into which they are introduced (for instance bacterial vectors having a bacterial origin of replication and episomal mammalian vectors). Other vectors (such as non-episomal mammalian vectors) may be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the present invention is intended to include such other forms of expression vectors, such as viral vectors (such as replication defective retroviruses, adenoviruses and adeno-associated viruses), which serve equivalent functions.

The term “recombinant host cell” (or simply “host cell”), as used herein, is intended to refer to a cell into which one or more expression vectors have been introduced. For example, the HC and LC of an antibody variant as described herein may both be encoded by the same expressing vector, and a host cell transfected with the expression vector. Alternatively, the HC and LC of an antibody variant as described herein may be encoded by different expression vectors, and a host cell co-transfected with the expression vectors. It should be understood that the term “host cell” is intended to refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. Recombinant host cells include, for example, transfectomas, such as CHO cells, HEK-293 cells, PER.C6, NS0 cells, and lymphocytic cells, and prokaryotic cells such as E. coli and other eukaryotic hosts such as plant cells and fungi.

The term “transfectoma”, as used herein, includes recombinant eukaryotic host cells expressing the Ab or a target antigen, such as CHO cells, PER.C6, NS0 cells, HEK-293 cells, plant cells, or fungi, including yeast cells.

The term “treatment” refers to the administration of an effective amount of a therapeutically active antibody variant of the present invention with the purpose of easing, ameliorating, arresting or eradicating (curing) symptoms or disease states.

The term “effective amount” or “therapeutically effective amount” refers to an amount effective, at dosages and for periods of time necessary, to achieve a desired therapeutic result. A therapeutically effective amount of an antibody may vary according to factors such as the disease state, age, sex, and weight of the individual, and the ability of the antibody to elicit a desired response in the individual. A therapeutically effective amount is also one in which any toxic or detrimental effects of the antibody variant are outweighed by the therapeutically beneficial effects.

### Specific Embodiments of the Invention

As described above, the present invention concerns antibodies that are variants of anti-CD38 antibody C, particularly those comprising a variant Fc region comprising a mutation in one or more amino acid residues selected from the group corresponding to E430, E345 and S440 in a human IgG1 heavy chain.

As shown in Example 3, CDC was enhanced for all three tested CD38 IgG1 antibodies—A, B and C—upon introduction of an E430G mutation. Surprisingly, however, the magnitude of CDC enhancement differed between the antibody clones tested. Without the E430G mutation, IgG1-B was already a good inducer of CDC, whereas IgG1-C and IgG1-A induced modest and no CDC respectively. Nonetheless, after introduction of the E430G mutation, however, IgG1-C-E430G induced more effective CDC compared to IgG1-B-E430G. In particular in tumor cells and T regulatory cells that have lower CD38 expression levels, EC50 values of IgG1-C-E430G were lower than those of IgG1-B-E430G.

Additionally, an antibody variant according to the invention may also demonstrate ADCC. For example, as shown in Example 4, IgG1-C achieved a higher maximum percent lysis as compared to IgG1-B in the ⁵¹Cr release assay and an increased FcγRIIIa binding in the ADCC reporter assay as compared to IgG1-B. Introduction of the E430G mutation reduced the maximum percent lysis in the ⁵¹Cr release assay and the FcγRIIIa binding in the ADCC reporter assay for all three antibodies. IgG1-C-E430G induced a similar maximum percent lysis as compared to IgG1-B-E430G and IgG1-A-E430G in the ⁵¹Cr release assay and similar FcγRIIIa binding in the ADCC reporter assay.

Moreover, the ability of an anti-CD38 antibody to inhibit CD38 cyclase activity can be retained in the form of an antibody variant according to the invention. For example, as shown in Example 7, IgG1-C-E430G displayed stronger inhibition of CD38 cyclase activity compared to IgG1-B-E430G, the former resulting in an inhibition of about 40% and the latter about 25%. Without being limited to theory, a stronger inhibition of CD38 cyclase activity may reduce production of cADPR, a potent second messenger that regulate Ca²⁺ mobilization from the cytosol, which in turn may lead to decreased Ca²⁺ mobilization and reduced signaling of downstream pathways that control various biological processes, such as proliferation and insulin secretion. Without being limited to theory, a stronger inhibition of CD38 cyclase activity may thus affect, e.g., reduce, the ability of immune suppressor cells to suppress an immune response.

Other functionalities that can be modulated include trogocytosis. Specifically, CD38 expression on Daudi cells was significantly reduced by co-culture with macrophages and CD38 antibody; however, the reduction in CD38 expression was strongest with E430G mutated antibody (Example 8). Surprisingly, CD38 expression on T regulatory cells co-cultured with PBMCs was only reduced after incubation with E430G-mutated CD38 antibody; no reduction in CD38 expression was found when T regulatory cells were incubated with antibody B. Without being limited to theory, the ability of antibody variants according to the present invention to induce trogocytosis of CD38-expressing, non-cancerous immune cells, particularly immunosuppressive cells, may in a cancer patient result in an increased immune response against tumor cells, irrespective of whether the tumor cells express CD38 or not.

The antibody variant of the present invention may also be able to kill tumor cells in vivo as shown in Example 9, where two weekly doses of IgG1-C-E430G reduced the tumor growth in two out of five tested DLBCL PDX models that had highest CD38 mRNA expression.

So, in one aspect, the invention provides an antibody variant binding to human CD38, the antibody variant comprising an antigen-binding region comprising the VH and VL CDRs of antibody C as set forth as SEQ ID NO:2 (VH-3003-C_CDR1), SEQ ID NO:3 (VH-3003-C_CDR2), SEQ ID NO:4 (VH-3003-C_CDR3), SEQ ID NO:6 (VL-3003-C_CDR1), AAS (VL-3003-C_CDR2) and SEQ ID NO:7 (VL-3003-C_CDR3) in Table 1, and a variant Fc region comprising a mutation in one or more amino acid residues selected from the group corresponding to E430, E345 and S440 in a human IgG1 heavy chain.

In one embodiment, the antibody variant binding to human CD38 comprises
(a) an antigen-binding region comprising a VH CDR1 having the sequence as set forth in SEQ ID NO:2, a VH CDR2 having the sequence as set forth in SEQ ID NO:3, a VH CDR3 having the sequence as set forth in SEQ ID NO:4, a VL CDR1 having the sequence as set forth in SEQ ID NO:6, a VL CDR2 having the sequence AAS, and a VL CDR3 having the sequence as set forth in SEQ ID NO:7, and
(b) a variant Fc region comprising a mutation in one or more amino acid residues selected from the group corresponding to E430, E345 and S440 in a human IgG1 heavy chain, wherein the amino acid residues are numbered according to the EU index.

In further embodiments, the antibody variant can also or alternatively be characterized by specific amino acid sequences or specific mutations in the antigen-binding region or Fc region and/or by its ability to induce effector functions or modulate CD38 enzyme activity. These are further described below.

### Antigen-Binding Region and Variable Regions

The antigen-binding region comprises one or more antibody variable domains allowing for specific binding to CD38, such as a VH region and a VL region. Similarly, the heavy and light chains comprise a VH and VL region, respectively. In the following reference to sequences in the antigen-binding region may similarly apply to sequences of the heavy and/or light chain of a variant antibody according to the present invention. Advantageously, the CDRs, VH region and/or VL region are similar or identical to those of antibody C, as set forth in Table 1.

In one preferred embodiment, the antigen-binding region, and/or the heavy and/or light chains comprise the CDRs of antibody C, set forth as SEQ ID NO:2 (VH-3003-C_CDR1), SEQ ID NO:3 (VH-3003-C_CDR2), SEQ ID NO:4 (VH-3003-C_CDR3), SEQ ID NO:6 (VL-3003-C_CDR1), AAS (VL-3003-C_CDR2) and SEQ ID NO:7 (VL-3003-C_CDR3). In another preferred embodiment, the VH and VL sequences are those of antibody C, i.e., the VH region comprises the sequence of SEQ ID NO:1 (VH-3003-C) and the VL region comprises the sequence of SEQ ID NO:5 (VL-3003-C).

However, it is well known in the art that mutations in the VH and VL of an antibody can be made to, for example, increase the affinity of an antibody to its target antigen, reduce its potential immunogenicity and/or to increase the yield of antibodies expressed by a host cell. Accordingly, in some embodiments, antibodies comprising variants of the CDR, VH and/or VL sequences of antibody C are also contemplated, particularly functional variants of the VL and/or VH region of antibody C. Functional variants may differ in one or more amino acids as compared to the parent VH and/or VL sequence, e.g., in one or more CDRs, but still allows the antigen-binding region to retain at least a substantial proportion (at least about 50 percent, 60 percent, 70 percent, 80 percent, 90 percent, 95 percent or more) of the affinity and/or specificity of the parent antibody. Typically, such functional variants retain significant sequence identity to the parent sequence. Exemplary variants include those which differ from the respective parent VH or VL region by 12 or less, such as 11, 10, 9, 8, 7, 6, 5, 4, 3, 2 or 1 mutation(s) such as substitutions, insertions or deletions of amino acid residues. Exemplary variants include those which differ from the VH and/or VL and/or CDR regions of the parent sequences mainly by conservative amino acid substitutions; for instance, 12, such as 11, 10, 9, 8, 7, 6, 5, 4, 3, 2 or 1 of the amino acid substitutions in the variant can be conservative. In some cases, an antibody comprising variants of the VH and/or VL of antibody C may be associated with greater affinity and/or specificity than the parent antibody. For the purpose of the present invention, VH and/or VL variants which allow for a retained or improved affinity and specificity of the antibody in its binding to CD38 are particularly preferred.

For example, WO 2011/154453 A1 discloses CD38 antibodies comprising suitable variant CDR, VH and VL region amino acid sequences, where the amino acid residues at certain positions differ from those in the CDRs, VH and VL of antibody C as shown in Table 1. These positions thus represent candidate positions where mutations in the CDR, VH and VL sequences can be made while retaining or improving affinity and specificity of the antibody in its binding to CD38. In particular, positions in the VH and VL CDRs that can be mutated in functional variants of the VH and VL of antibody C are indicated in SEQ ID NOS:40 to 43.

So, in some embodiments, one or more specific mutations are made in the CDRs as set forth in SEQ ID NOS:40 to 43, i.e., any functional variants of the VH and/or VL region comprises mutations in the CDRs as set forth in one or more of SEQ ID NO:40 (VH CDR1), SEQ ID NO:41 (VH CDR2), SEQ ID NO:42 (VH CDR3), and SEQ ID NO:44 (VL CDR3). The VH and VL regions of such an antibody variant may optionally maintain the original framework regions of antibody C. In one specific embodiment, the antigen-binding region comprises the CDRs as set forth in SEQ ID NO:40 wherein X₁ is S (VH CDR1), SEQ ID NO:41 wherein X₁ is R, X₂ is K, X₃ is A (VH CDR2), SEQ ID NO:42 wherein X₁ is A, X₂ is D and X₃ is V (VH CDR3), SEQ ID NO:43 (VL CDR1), AAS (VL CDR2) and SEQ ID NO:44 wherein X₁ is S (VL CDR3). In one specific embodiment, the antigen-binding region comprises the CDRs as set forth in SEQ ID NO:40 wherein X₁ is R (VH CDR1), SEQ ID NO:41 wherein X₁ is V, X₂ is K, X₃ is T (VH CDR2), SEQ ID NO:42 wherein X₁ is T, X₂ is A and X₃ is F (VH CDR3), SEQ ID NO:43 (VL CDR1), AAS (VL CDR2) and SEQ ID NO:44 wherein X₁ is N (VL CDR3). In one specific embodiment, the antigen-binding region comprises the CDRs as set forth in SEQ ID NO:40 wherein X₁ is S (VH CDR1), SEQ ID NO:41 wherein X₁ is R, X₂ is K, X₃ is T (VH CDR2), SEQ ID NO:42 wherein X₁ is A, X₂ is D and X₃ is V (VH CDR3), SEQ ID NO:43 (VL CDR1), AAS (VL CDR2) and SEQ ID NO:44 wherein X₁ is S (VL CDR3). In one specific embodiment, the antigen-binding region comprises the CDRs as set forth in SEQ ID NO:40 wherein X₁ is R (VH CDR1), SEQ ID NO:41 wherein X₁ is V, X₂ is K, X₃ is V (VH CDR2), SEQ ID NO:42 wherein X₁ is T, X₂ is A and X₃ is F (VH CDR3), SEQ ID NO:43 (VL CDR1), AAS (VL CDR2) and SEQ ID NO:44 wherein X₁ is N (VL CDR3).

In some embodiments, no mutation is made in the CDRs, i.e., any functional variants of the VH and/or VL region retains the CDR sequences set forth in SEQ ID NO:2, SEQ ID NO:3, SEQ ID NO:4 or SEQ ID NO:6, AAS, SEQ ID NO:7, respectively representing the VH CDR1-3 or VL CDR1-3 sequences of antibody C.

In one embodiment, the VH region comprises SEQ ID NO:1 or an amino acid sequence having at least 80% identity, such as 90%, or 95%, or 97%, or 98%, or 99%, to SEQ ID NO:1. For example, the VH may differ from SEQ ID NO:1 by 12 or less, such as 11, 10, 9, 8, 7, 6, 5, 4, 3, 2 or 1 mutations such as substitutions, insertions or deletions of amino acid residues. In one embodiment, the VH region differs from SEQ ID NO:1 only in 12 or less, such as 5 or less, such as 5, 4, 3, 2 or 1 amino acid substitutions. The amino acid substitutions may, for example, be conservative amino acid substitutions as described elsewhere herein. In a particular embodiment, no mutation is made in the VH CDRs, i.e., any variant VH retains the C CDR sequences set forth in SEQ ID NO:2, SEQ ID NO:3, SEQ ID NO:4.

In one embodiment, the VL region comprises SEQ ID NO:5 or an amino acid sequence having at least 80% identity, such as 90%, or 95%, or 97%, or 98%, or 99%, to SEQ ID NO:5. For example, the VL may differ from SEQ ID NO:5 by 12 or less, such as 11, 10, 9, 8, 7, 6, 5, 4, 3, 2 or 1 mutations such as substitutions, insertions or deletions of amino acid residues. In one embodiment, the VL region differs from SEQ ID NO:5 only in 12 or less, such as 5 or less, such as 5, 4, 3, 2 or 1 amino acid substitutions. The amino acid substitutions may, for example, be conservative amino acid substitutions as described elsewhere herein. In a particular embodiment, no mutation is made in the VL CDRs, i.e., any variant VH retains the C CDR sequences set forth in SEQ ID NO:6, AAS, SEQ ID NO:7.

In one embodiment, the antibody variant comprises a VH region comprising the sequence of SEQ ID NO:1 and a VL region comprising the sequence of SEQ ID NO:5.

### Variant Fc Region, and CH Region

Mutations in amino acid residues at positions corresponding to E430, E345 and S440 in a human IgG1 heavy chain, wherein the amino acid residues are numbered according to the EU index, can improve the ability of an antibody to induce CDC (see, e.g., Example 3). Without being bound by theory, it is believed that by substituting one or more amino acid(s) in these positions, oligomerization of the antibody can be stimulated, thereby modulating effector functions so as to, e.g., increase C1q binding, complement activation, CDC, ADCP, internalization or other relevant function(s) that may provide in vivo efficacy.

The present invention relates to a variant antibody comprising an antigen-binding region and a variant Fc region.

In certain embodiments, an antibody variant binding to human CD38 comprises

(a) a heavy chain comprising a VH region comprising a VH CDR1 having the sequence as set forth in SEQ ID NO:2, a VH CDR2 having the sequence as set forth in SEQ ID NO:3, a VH CDR3 having the sequence as set forth in SEQ ID NO:4 and a human IgG1 CH region with a mutation in one or more of E430, E345 and S440, the amino acid residues being numbered according to the EU index;
(b) a light chain comprising a VL region comprising a VL CDR1 having the sequence as set forth in SEQ ID NO:6, a VL CDR2 having the sequence AAS, and a VL CDR3 having the sequence as set forth in SEQ ID NO:7.

In other certain embodiments, an antibody variant binding to human CD38 comprises

(a) a heavy chain comprising a VH region comprising SEQ ID NO:1 and a human IgG1 CH region with a mutation in one or more of E430, E345 and S440, the amino acid residues being numbered according to the EU index, and
(b) a light chain comprising a VL region comprising SEQ ID NO:5.

A variant antibody of the present invention comprises a variant Fc region or a human IgG1 CH region comprising a mutation in one or more of E430, E345 and S440. In the following reference to the mutations in the Fc region may similarly apply to the mutation(s) in the human IgG1 CH region.

As described herein, the position of an amino acid to be mutated in the Fc region can be given in relation to (i.e., “corresponding to”) its position in a naturally occurring (wild-type) human IgG1 heavy chain, when numbered according to the EU index. So, if the parent Fc region already contains one or more mutations and/or if the parent Fc region is, for example, an IgG2, IgG3 or IgG4 Fc region, the position of the amino acid corresponding to an amino acid residue such as, e.g., E430 in a human IgG1 heavy chain numbered according to the EU index can be determined by alignment. Specifically, the parent Fc region is aligned with a wild-type human IgG1 heavy chain sequence so as to identify the residue in the position corresponding to E430 in the human IgG1 heavy chain sequence. Any wild-type human IgG1 constant region amino acid sequence can be useful for this purpose, including any one of the different human IgG1 allotypes set forth in Table 1. This is illustrated in FIG. 1, which shows an alignment between two different human IgG1 allotypes—IgG1m(f) and IgG1m(a)—and wild-type human IgG2, IgG3 and IgG4, specifically of the segments corresponding to residues P247 to K447 in a human IgG1 heavy chain, wherein the amino acid residues are numbered according to the EU index.

Accordingly, in the remaining paragraphs of this section and elsewhere herein, unless otherwise specified or contradicted by context, the amino acid positions referred to are those corresponding to amino acid residues in a wild-type human IgG heavy chain, wherein the amino acid residues are numbered according to the EU index:

In separate and specific embodiments, the variant Fc region and/or the human IgG1 CH region comprises a mutation in only one of E430, E345 and S440; in both E430 and E345; in both E430 and S440; in both E345 and S440; or in all of E430, E345 and S440. In some embodiments, the variant Fc region and/or the human IgG1 CH region comprises a mutation in only one of E430, E345 and S440; in both E430 and E345; in both E430 and S440; in both E345 and S440; or in all of E430, E345 and S440, with the proviso that any mutation in S440 is S440W or S440Y. In other separate and specific embodiments, the mutation is an amino acid substitution. In one embodiment the mutation is an amino acid substitution in only one of E430X, E345X and S440X; in both E430X and E345X; in both E430X and S440X; in both E345X and S440X; or in all of E430X, E345X and S440X, preferably with the proviso that any mutation in S440X is S440Y or S440W. More preferably, the E430X, E345X and S440X mutations are separately selected from E430G, E345K, E430S, E430F, E430T, E345Q, E345R, E345Y, S440Y and S440W.

In one embodiment, the mutation in the one or more amino acid residues is selected from the group consisting of E430G, E345K, E430S, E430F, E430T, E345Q, E345R, E345Y, S440Y and S440W.

In a preferred embodiment, the mutation in the one or more amino acid residues is selected from the group corresponding to E430G, E345K, E430S and E345Q.

In one embodiment, the mutation is in an amino acid residue corresponding to E430, such as an amino acid substitution, E430X, e.g., selected from those corresponding to E430G, E430S, E430F, or E430T. In one preferred embodiment, the mutation in the one or more amino acid residues comprises E430G. In another preferred embodiment, the mutation in the one or more amino acid residues comprises E430S, optionally wherein no mutations are made in the amino acid residues corresponding to E345 and S440. In a particularly preferred embodiment, the mutation in the one or more amino acid residue consists of E430G, i.e., no mutations are made in the amino acid residues corresponding to E345 and S440.

In one embodiment, the mutation is in an amino acid residue corresponding to E345, such as an amino acid substitution, E345X, e.g., selected from those corresponding to E345K, E345Q, E345R and E345Y. In one preferred embodiment, the mutation in the one or more amino acid residues comprises E345K. In another preferred embodiment, the mutation in the one or more amino acid residues comprises E345Q, optionally wherein no mutations are made in the amino acid residues corresponding to E430 and S440. In a particularly preferred embodiment, the mutation in the one or more amino acid residue consists of E345K, i.e., no mutations are made in the amino acid residues corresponding to E430 and S440.

In one embodiment, the mutation is in an amino acid residue corresponding to S440, such as an amino acid substitution, S440X, typically selected from those corresponding to S440Y and S440W. In one preferred embodiment, the mutation in the one or more amino acid residues comprises S440W, optionally wherein no mutations are made in the amino acid residues corresponding to E430 and E345. In one preferred embodiment, the mutation in the one or more amino acid residues comprises S440Y, optionally wherein no mutations are made in the amino acid residues corresponding to E430 and E345.

Preferably, the antibody variant comprises a variant Fc region according to any one of the preceding sections, which variant Fc region is a variant of a human IgG Fc region selected from the group consisting of a human IgG1, IgG2, IgG3 and IgG4 Fc region. That is, the mutation in one or more amino acid residues corresponding to E430, E345 and S440 is/are made in a parent Fc region which is a human IgG Fc region selected from the group consisting of an IgG1, IgG2, IgG3 and IgG4 Fc region. Preferably, the parent Fc region is a naturally occurring (wild-type) human IgG Fc region, such as a human wild-type IgG1, IgG2, IgG3 or IgG4 Fc region, or a mixed isotype thereof. Thus, the variant Fc region may, except for the recited mutation (in the one or more amino acid residues selected from the group corresponding to E430, E345 and S440), be a human IgG1, IgG2, IgG3 or IgG4 isotype, or a mixed isotype thereof.

In one embodiment, the parent Fc region and/or human IgG1 CH region is a wild-type human IgG1 isotype.

Thus, the variant Fc region may except for the recited mutation (in the one or more amino acid residues selected from the group corresponding to E430, E345 and S440), be a human IgG1 Fc region.

In a specific embodiment, the parent Fc region and/or human IgG1 CH region is a human wild-type IgG1m(f) isotype.

In a specific embodiment, the parent Fc region and/or human IgG1 CH region is a human wild-type IgG1m(z) isotype.

In a specific embodiment, the parent Fc region and/or human IgG1 CH region is a human wild-type IgG1m(a) isotype.

In a specific embodiment, the parent Fc region and/or human IgG1 CH region is a human wild-type IgG1m(x) isotype.

In a specific embodiment, the parent Fc region and/or human IgG1 CH region is a human wild-type IgG1 of a mixed allotype, such as IgG1m(za), IgG1m(zax), IgG1m(fa), or the like.

Thus, the variant Fc region and/or human IgG1 CH region may, except for the recited mutation (in the one or more amino acid residues selected from the group corresponding to E430, E345 and S440), be a human IgG1m(f), IgG1m(a), IgG1m(x), IgG1m(z) allotype or a mixed allotype of any two or more thereof.

In a specific embodiment, the parent Fc region and/or human IgG1 CH region is a human wild-type IgG1m(za) isotype.

In a specific embodiment, the parent Fc region is a human wild-type IgG2 isotype.

In a specific embodiment, the parent Fc region is a human wild-type IgG3 isotype.

In a specific embodiment, the parent Fc region is a human wild-type IgG4 isotype.

CH region amino acid sequences of specific examples of wild-type human IgG isotypes and IgG1 allotypes are set forth in Table 1. In some embodiments, the parent Fc region comprises the CH2-CH3 or, optionally, the hinge-CH2-CH3 segments of such wild-type CH region amino acid sequences.

So, in a specific embodiment, the parent Fc region is a human wild-type IgG1 isotype comprising the amino acid residues corresponding to 231-447 in a human IgG1 heavy chain according to the EU numbering. For example, the parent Fc region may comprise amino acid residues 114 to 330 (direct numbering) of a sequence selected from the group consisting of SEQ ID NO:19, SEQ ID NO:20, SEQ ID NO:21, SEQ ID NO:22 and SEQ ID NO:23 In a specific embodiment, the parent Fc region is a human wild-type IgG1 isotype comprising the amino acid residues corresponding to 216-447 in a human IgG1 heavy chain according to the EU numbering. For example, the parent Fc region may comprise amino acid residues 99 to 330 (direct numbering) of a sequence selected from the group consisting of SEQ ID NO:19, SEQ ID NO:20, SEQ ID NO:21, SEQ ID NO:22 and SEQ ID NO:23. As described elsewhere herein for production of therapeutic antibodies, the C-terminal amino acid K447 may sometimes be deleted or removed. Hence the parent Fc region may comprise amino acid residues 114 to 329 (direct numbering) or amino acid residues 99 to 329 (direct numbering) of SEQ ID NO: 45.

In a specific embodiment, the variant Fc region is a variant of a human wild-type IgG1 isotype comprising the amino acid residues corresponding to 231-447 in a human IgG1 heavy chain according to the EU numbering. For example, the variant Fc region may comprise amino acid residues 114 to 330 (direct numbering) of a sequence selected from the group consisting of SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32 and SEQ ID NO:33. In another embodiment the variant Fc region may comprise amino acid residues 114 to 329 (direct numbering) of SEQ ID NO: 46.

In a specific embodiment, the variant Fc region is a variant of a human wild-type IgG1 isotype comprising the amino acid residues corresponding to 216-447 in a human IgG1 heavy chain according to the EU numbering. For example, the variant Fc region may comprise amino acid residues 99 to 330 (direct numbering) of a sequence selected from the group consisting of SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32 and SEQ ID NO:33. In another embodiment the variant Fc region may comprise amino acid residues 99 to 329 (direct numbering) of SEQ ID NO: 46.

So, the present invention can be applied to antibody molecules having a human IgG1 heavy chain, such as a human IgG1 heavy chain comprising a human IgG1 CH region amino acid sequence comprising SEQ ID NO:19 (IgGm(za). Thus, the human IgG1 CH region may comprise, except for the recited mutation, the sequence of SEQ ID NO:19.

The present invention can also be applied to antibody molecules having a human IgG1 heavy chain, such as a human IgG1 heavy chain comprising a human IgG1 CH region amino acid sequence comprising SEQ ID NO:20 (IgGm(f)) or SEQ ID NO: 45. Thus, the human IgG1 CH region may comprise, except for the recited mutation, the sequence of SEQ ID NO:20. In another embodiment the human IgG1 CH region may comprise, except for the recited mutation, the sequence of SEQ ID NO: 45.

The present invention can also be applied to antibody molecules having a human IgG1 heavy chain, such as a human IgG1 heavy chain comprising a human IgG1 CH region amino acid sequence comprising SEQ ID NO:21 (IgGm(z)). Thus, the human IgG1 CH region may comprise, except for the recited mutation, the sequence of SEQ ID NO:21.

The present invention can also be applied to antibody molecules having a human IgG1 heavy chain, such as a human IgG1 heavy chain comprising a human IgG1 CH region amino acid sequence comprising, SEQ ID NO:22 (IgGm(a)). Thus, the human IgG1 CH region may comprise, except for the recited mutation, the sequence of SEQ ID NO:22.

The present invention can also be applied to antibody molecules having a human IgG1 heavy chain, such as a human IgG1 heavy chain comprising a human IgG1 CH region amino acid sequence comprising SEQ ID NO:23 (IgG1m(x)). Thus, the human IgG1 CH region may comprise, except for the recited mutation, the sequence of SEQ ID NO:23.

In other separate and specific embodiments, the human IgG1 CH region comprises an amino acid sequence selected from the group consisting of SEQ ID NO:24 to SEQ ID NO:33 and SEQ ID NO: 45.

In a specific embodiment, the human IgG1 CH region comprises SEQ ID NO:24 (IgG1m(f)-E430G) or SEQ ID NO:46, optionally wherein the light chain comprises a CL comprising SEQ ID NO:37.

In a specific embodiment, the antibody variant is a monospecific antibody comprising two HCs that are identical in amino acid sequence and two LCs that are identical in amino acid sequence.

The present invention can also be applied to antibody molecules having a human IgG2 heavy chain, such as a human IgG2 heavy chain comprising a human IgG2 CH region amino acid sequence comprising SEQ ID NO:34.

The present invention can also be applied to antibody molecules having a human IgG3 heavy chain, such as a human IgG3 heavy chain comprising a human IgG3 CH region amino acid sequence comprising SEQ ID NO:35.

The present invention can also be applied to antibody molecules having a human IgG4 heavy chain, such as a human IgG4 heavy chain comprising a human IgG4 CH region amino acid sequence comprising SEQ ID NO:36.

However, variant Fc regions comprising one or more further mutations, i.e., mutations in one or more other amino acid residues other than those corresponding to E430, E345 and S440 in a human IgG1 heavy chain when numbered according to the EU index, are also contemplated for the antibody variants disclosed herein. Also or alternatively, the Fc region may be a mixed isotype, e.g., where different CH regions derive from different IgG isotypes. Accordingly, as described in more detail below, the parent Fc region may already comprise one or more further mutations as compared to such a wild-type (naturally occurring) human IgG Fc region, or may be a mixed isotype.

In one embodiment, the parent Fc region into which a mutation in one or more amino acid residues selected from the group corresponding to E430, E345 and S440 is introduced, is a human IgG Fc region which comprises one or more further mutations as compared to a wild-type human IgG1, IgG2, IgG3 and IgG4 Fc region, e.g., as set forth in one of SEQ ID NO:19, SEQ ID NO:20, SEQ ID NO:21, SEQ ID NO:22, SEQ ID NO:23, SEQ ID NO:34, SEQ ID NO:35 and SEQ ID NO:36. Expressed in an alternative manner, the variant Fc region comprising a mutation in E430, E345 and/or S440 may differ also in one or more further mutations from a reference Fc region, such as a reference wild-type human IgG1, IgG2, IgG3 and IgG4 Fc region, e.g., as set forth in one of SEQ ID NO:19, SEQ ID NO:20, SEQ ID NO:21, SEQ ID NO:22, SEQ ID NO:23, SEQ ID NO:34, SEQ ID NO:35 and SEQ ID NO:36. For example, except for the mutation in one or more amino acid residues selected from the group corresponding to E430, E345 and S440, the variant Fc region may differ from the wild-type Fc region by 12 or less, such as 11, 10, 9, 8, 7, 6, 5, 4, 3, 2 or 1 mutations such as substitutions, insertions or deletions of amino acid residues. For example the C-terminal amino acid Lys (K) at position 447 (Eu numbering) may have been deleted. Some host cells which are used for production of an antibody may contain enzymes capable of removing the Lys at position 447, and such removal may not be homogenous. Therapeutic antibodies may therefore be produced without the C-terminal Lys (K) to increase the homogenicity of the product. Methods for producing antibodies without the C-terminal Lys (K) are well-known to a person skilled in the art and include genetic engineering of the nucleic acid expressing said antibody, enzymatic methods and use of specific host cells. Thus, for example the parent Fc region may comprise the sequence as set forth in SEQ ID NO: 45.

Preferably, any such one or more further mutations do not reduce the ability of the antibody as disclosed herein, i.e., an antibody comprising a mutation in one or more amino acid residues selected from the group corresponding to E430, E345 and S440 in a human IgG1 heavy chain, to induce CDC and/or ADCC. More preferably, any such one or more further mutations do not reduce the ability of the antibody to induce CDC. Most preferably, any such one or more further mutations do not reduce the ability of the antibody to induce either one of CDC and ADCC. Candidates for the one or more further mutations can, for example, be tested in CDC or ADCC assays, e.g., as disclosed herein, such as in Examples 3 and 4. For example, the CDC of an antibody as described herein, e.g., IgG1-C-E430G, can be tested in the assay of Example 3 or an assay as described in the next section (or a similar assay) with and without specific candidates for one or more further mutations, so as to ascertain the effect of the candidate further mutation(s) on the ability of the antibody to induce CDC. Likewise, the ADCC of an antibody as described herein, e.g., IgG1-C-E430G, can be tested in the assay of Example 4 or an assay as described in the next section (or a similar assay) with and without a specific candidate for a further mutation so as to ascertain the effect of the candidate further mutation on the ability on the antibody to induce ADCC.

Preferably, in an antibody variant comprising two HCs and two LCs, the Fc regions in the first and second HC are identical such that the Fc region, in dimerized form, is a homodimer.

However, in some embodiments, in an antibody variant comprising two HCs and two LCs, the Fc region in the first HC may differ in one or more amino acids from the Fc region in the second HC, such that the Fc region, in dimerized form, is a heterodimer. For example, the mutation in one or more amino acid residues selected from the group corresponding to E430, E345 and S440 in an IgG1 heavy chain, wherein the amino acid residues are numbered according to the EU index, may only be present in one of the Fc regions. Accordingly, in some embodiments, one Fc region may be SEQ ID NO:45 or a human wild-type IgG Fc region selected from SEQ ID NO:19, SEQ ID NO:20, SEQ ID NO:21, SEQ ID NO:22, SEQ ID NO:23, SEQ ID NO:34, SEQ ID NO:35 and SEQ ID NO:36 while the other Fc region may be identical except for a mutation in said one or more amino acid residues selected from the group corresponding to E430, E345 and S440 in an IgG1 heavy chain.

In one embodiment, the antibody variant according to any aspect or embodiment herein is, except for the recited mutations, a human antibody.

In one embodiment, the antibody variant according to any aspect or embodiment herein is, except for the recited mutations, a full-length antibody, such as a human full-length antibody.

In one embodiment, the antibody variant according to any aspect or embodiment herein is, except for the recited mutations, a bivalent antibody, such as a human bivalent antibody, such as a human bivalent full-length antibody.

In one embodiment, the antibody variant according to any aspect or embodiment herein is, except for the recited mutations, a monoclonal antibody, such as a human monoclonal antibody, such as a human bivalent monoclonal antibody, such as a human bivalent full-length monoclonal antibody.

In a preferred embodiment, the antibody variant according to any aspect or embodiment herein is, except for the recited mutations, an IgG1 antibody, such as a full length IgG1 antibody, such as a human full-length IgG1 antibody, optionally a human monoclonal full-length bivalent IgG1,κ antibody, e.g. a human monoclonal full-length bivalent IgG1m(f),κ antibody.

An antibody variant according to the present invention is advantageously in a bivalent monospecific format, comprising two antigen-binding regions binding to the same epitope. However, bispecific formats where one of the antigen-binding regions binds to a different epitope are also contemplated. So, the antibody variant according to any aspect or embodiment herein can, unless contradicted by context, be either a monospecific antibody or a bispecific antibody.

So, in one embodiment, the antibody variant according to any aspect or embodiment herein is, except for the recited mutations, a monospecific antibody, such as a human monospecific antibody, such as a human full-length monospecific antibody, such as a human full-length monospecific bivalent monoclonal antibody, such as a human full-length bivalent monospecific monoclonal antibody.

In another embodiment, the antibody variant according to any aspect or embodiment herein is, except for the recited mutations, a bispecific antibody, such as a full-length bispecific antibody, optionally a full-length bispecific and bivalent IgG1,κ antibody.

### Modulation of Functions

The antibody variant according to any aspect or embodiment herein can typically induce one or more, preferably all, of CDC, ADCC, ADCP, apoptosis in the presence but not absence of an Fc-cross-linking agent, trogocytosis, or any combination thereof, of target cells expressing human CD38, typically in the presence of complement and effector cells.

The antibody variant according to any aspect or embodiment herein may typically modulate the enzyme activity of CD38.

In a further embodiment the antibody variant according to any aspect or embodiment herein may induce one or more of CDC, ADCC, ADCP, apoptosis in the presence but not absence of an Fc-cross-linking agent, trogocytosis, and modulate the enzyme activity of CD38, or any combination thereof.

### Complement-Dependent Cytotoxicity (CDC):

In one embodiment, the antibody variant as disclosed herein induces CDC. In particular, the antibody variants of the present invention may mediate an increased CDC when bound to CD38 on, for example, the surface of a CD38-expressing cell or cell-membrane, as compared to a control. The control can be, for example, a reference antibody with amino acid sequences (typically heavy- and light chain amino acid sequences) identical to the antibody variant except for the one or more mutations in E430, E345 and/or S440 in the variant antibody. Alternatively, the control can be a reference antibody with amino acid sequences (typically heavy- and light chain amino acid sequences) identical to the antibody variant except for different VH and VL sequences. Such a reference antibody could, for example, instead have the VH and VL sequences of antibody B or A, as shown in Table 1. Preferably, the VH and VL sequences of the reference antibody are those of antibody B. Alternatively, the reference antibody may be an antibody binding the same target but with different amino acid sequences. Alternatively, the control may be an isotype control antibody, e.g., such that the VH and VL sequences are those of antibody b12 as shown in Table 1.

Accordingly, in one embodiment, the antibody variant according to any aspect or embodiment disclosed herein induces a higher CDC against CD38-expressing target cells than a reference antibody, wherein the reference antibody comprises the VH and VL region sequences of antibody C, i.e., SEQ ID NO:1 and SEQ ID NO:5, respectively, and CH and CL region sequences identical to the antibody variant except for the one or more mutations in E430, E345 and/or S440.

In another embodiment, the antibody variant according to any aspect or embodiment disclosed herein induces a higher CDC against CD38-expressing target cells than a reference antibody, wherein the reference antibody comprises the VH and VL region sequences of antibody C, i.e., SEQ ID NO:1 and SEQ ID NO:5, respectively, and the CH and CL region sequences of SEQ ID NO:20 (IgGm(f)) and SEQ ID NO:37 (kappa), respectively.

In another embodiment, the antibody variant according to any aspect or embodiment disclosed herein induces a higher CDC against CD38-expressing target cells than a reference antibody, wherein the reference antibody comprises the VH and VL region sequences of antibody B, i.e., SEQ ID NO:8 and SEQ ID NO:9, respectively, and CH and CL region sequences identical to the antibody variant.

In another embodiment, the antibody variant according to any aspect or embodiment disclosed herein induces a higher CDC against CD38-expressing target cells than a reference antibody, wherein the reference antibody comprises the VH and VL region sequences of antibody A, i.e., SEQ ID NO:10 and SEQ ID NO:11, respectively, and CH and CL region sequences identical to the antibody variant.

In another embodiment, the antibody variant according to any aspect or embodiment disclosed herein induces a higher CDC against CD38-expressing target cells than a reference antibody, wherein the reference antibody comprises the VH and VL region sequences of antibody b12, i.e., SEQ ID NO:12 and SEQ ID NO:16, respectively, and CH and CL region sequences identical to the antibody variant.

In one specific embodiment, the CDC response is described as maximum lysis, where a higher maximum lysis reflects an increased CDC. In one specific embodiment, the CDC response is described as EC50 (the concentration at which half maximal lysis is observed), where a lower EC50 indicates an increased CDC. In one specific embodiment, the CD38-expressing target cells are tumor cells, such as lymphoma cells. Non-limiting examples of lymphoma target cells include (indicating, within parentheses, a commercial source):
Daudi cells (ATCC CCL-213);
Ramos cells (ATCC CRL-1596);
REH cells (DSMZ ACC 22);
Wien-133 cells (BioAnaLab, Oxford, U.K.);
RS4; 11 cells (DSMZ ACC 508);
NALM-16 (DSMZ ACC 680);
U266 (ATCC TIB-196);
RC-K8 (DSMZ ACC 561);
SU-DHL-8;
Oci-Ly-7;
Oci-Ly-19;
Oci-Ly-18;
Raj i;
DOHH-2;
SU-DHL-4;
WSU-DLCL-2;
Z-138;
JVM-13;
Jeko-1;
697;
Granta 519;
DB;
Pfeiffer.

The CD38-expressing target cells may also be an AML cell, such as one selected from the consisting of but not limited to: THP1, monomac6, Oci-AML3, KG-1, ML2, U937, Nomo-1, AML-193, MEGAL, MOLM13, HL-60 and Oci-M1.

In another specific embodiment, the CD38-expressing target cells are tumor cells, such as lymphoma cells or myeloma cells, wherein the approximate average number of CD38 molecules per cell is in one of the following ranges, optionally when determined as described in Example 1:
150,000-250,000, such as about 200,000;
200,000-300,000, such as about 260,000;
80,000-180,000, such as about 130,000;
50,000-150,000, such as about 100,000;
40,000-120,000, such as about 80,000;
30,000-70,000, such as about 50,000;
10,000-20,000, such as about 15,000;
5,000-15,000, such as about 10,000.

In one embodiment, the antibody variant according to any aspect or embodiment as disclosed here induces an increased CDC against CD38-expressing target cells as compared to a reference antibody, wherein the reference antibody comprises the VH and VL region sequences of antibody B, i.e., SEQ ID NO:8 and SEQ ID NO:9, respectively, and CH and CL region sequences identical to the antibody variant, wherein the CDC-response is EC50 and the CD38-expressing target cells are selected from NALM-16 (DSMZ ACC 680), U266 (ATCC TIB-196) and RC-K8 (DSMZ ACC 561).

In a preferred embodiment, the antibody variant according to any aspect or embodiment as disclosed here induces an increased CDC against CD38-expressing target cells as compared to a reference antibody, wherein the reference antibody comprises the VH and VL region sequences of antibody C, i.e., SEQ ID NO:1 and SEQ ID NO:5, respectively and the CH and CL region sequences of SEQ ID NO:20 (IgGm(f)) and SEQ ID NO:37 (kappa), respectively, wherein the CDC-response is maximum lysis and the CD38-expressing target cells are selected from Daudi cells (ATCC CCL-213) and Ramos cells (ATCC CRL-1596). The antibody variant may in particular result in at least 50%, such as at least 60% or at least 70% higher maximum lysis than the reference antibody.

Any in vitro or in vivo method or assay known by the skilled person and suitable for evaluating the ability of an antibody, such as an IgG antibody, to induce CDC against CD38-expressing target cells can be used. Preferably, the assay comprises, in relevant part, the steps of the CDC assay described in Example 3.

A non-limiting example of an assay for determining the maximum lysis of CD38 expressing cells as mediated by a CD38 antibody, or the EC50 value, may comprise the steps of:
(a) plating about 100,000 CD38-expressing cells in 40 μL culture medium supplemented with 0.2% BSA per well in a multi-well plate;
(b) preincubating cells for 20 minutes with 40 μL of serially diluted CD38 antibody (0.0002-10 μg/mL);
(c) incubating each well for 45 minutes at 37° C. with 20 percent of pooled normal human serum;
(d) adding a viability dye and measuring the percentage of cell lysis on a flow cytometer;
(e) determining the maximum lysis and/or calculating the EC50 value using non-linear regression.

Tumor cells suitable for this assay include, without limitation, those listed in Table 2, such as Daudi cells (ATCC CCL-213).

In certain embodiments, the antibody variant induces CDC against Daudi cells (ATCC No. CCL-213) or Ramos cells (ATCC No. CRL-1596) resulting in a maximum lysis at least 50%, such at least 60%, such as at least 70% higher than that obtained with a reference antibody differing only in the absence of the mutation in the one or more amino acid residues selected from the group corresponding to E430, E435 and S440 in a human IgG1 heavy chain, wherein the amino acid residues are numbered according to the EU index. In one embodiment, the reference antibody comprises the VH and VL region sequences of antibody C, i.e., SEQ ID NO:1 and SEQ ID NO:5, respectively and the CH and CL region sequences of SEQ ID NO:20 (IgGm(f)) and SEQ ID NO:37 (kappa), respectively.

### Antibody-Dependent Cell-Mediated Cytotoxicity (ADCC):

In one embodiment, the antibody variant according to any aspect or embodiment herein induces ADCC. In some embodiments, the antibody variants of the present invention may mediate ADCC when bound to CD38 on, for example, the surface of a CD38-expressing cell or cell membrane. The anti-CD38 antibodies comprising an E430G mutation were found to induce slightly lower levels of ADCC compared to the same antibody without an E430G mutation. The antibody variants of the present invention may mediate higher ADCC when bound to CD38 on, for example, the surface of a CD38-expressing cell or cell membrane, than a control, wherein he control can be, for example, a reference antibody with amino acid sequences (typically heavy- and light chain amino acid sequences) identical to the antibody variant except for different VH and VL sequences. Such a reference antibody could, for example, instead have the VH and VL sequences of antibody B or A, as shown in Table 1. Preferably, the VH and VL sequences of the reference antibody are those of antibody B. Alternatively, the control may be an isotype control antibody, e.g., such that the VH and VL sequences are those of antibody b12 as shown in Table 1.

Accordingly, in one embodiment, the antibody variant according to any aspect or embodiment disclosed herein, induces a higher ADCC against CD38-expressing target cells than a reference antibody, wherein the reference antibody comprises the VH and VL region sequences of antibody B, i.e., SEQ ID NO:8 and SEQ ID NO:9, respectively and CH and CL region sequences identical to the antibody variant. In one specific embodiment, the ADCC response is maximum lysis, where a higher maximum lysis reflects a higher ADCC. In one specific embodiment, the ADCC response evaluated in an assay determining FcγRIIIa binding, where a higher binding indicates a higher ADCC. In one specific embodiment, the CD38-expressing target cells are tumor cells. Non-limiting examples of target cells include Daudi, Wien-133, Granta 519, MEC-2 and the tumor cell lines listed in Table 2.

In one embodiment, the antibody variant according to any aspect or embodiment disclosed herein induces a higher ADCC against CD38-expressing Daudi cells as compared to a reference antibody, wherein the reference antibody comprises the VH and VL region sequences of antibody B, i.e., SEQ ID NO:8 and SEQ ID NO:9, respectively and CH and CL region sequences identical to the antibody variant, optionally wherein the ADCC response is maximum lysis or FcγRIIIa binding.

In one embodiment, the antibody variant according to any aspect or embodiment disclosed herein induces a higher ADCC against CD38-expressing Daudi cells as compared to a reference antibody, wherein the reference antibody comprises the VH and VL region sequences of antibody b12, i.e., SEQ ID NO:12 and SEQ ID NO:16, respectively and CH and CL region sequences identical to the antibody variant, optionally wherein the ADCC response is maximum lysis or FcγRIIIa binding.

Any in vitro or in vivo method or assay known by the skilled person and suitable for evaluating the ability of an antibody, such as an IgG antibody, to induce ADCC against CD38-expressing target cells can be used. Preferably, the assay comprises, in relevant part, the steps of the ⁵¹Cr-release antibody-dependent cellular cytotoxicity assay or the ADCC reporter bioassay described in Example 4. Non-limiting examples of assays for determining the ADCC of CD38-expressing cells as mediated by a CD38 antibody may comprise the steps of the 51Cr-release assay or the reporter assay set out below.

ADCC with ⁵¹Cr release:
(a) plating about 5,000 ⁵¹Cr labelled CD38-expressing cells (e.g., Daudi cells) in 50 μL culture medium supplemented with 0.2% BSA per well in a multi-well plate;
(b) preincubating cells for 15 minutes with 50 μL of serially diluted CD38 antibody (0.0002-10 μg/mL);
(c) incubating each well for 4 hours at 37° C. with 500,000 freshly isolated peripheral blood mononuclear cells (PBMCs) per well;
(d) measuring the amount of ⁵¹Cr release in 75 μL supernatant on a gamma counter;
(e) calculating the percentage of cell lysis as (cpm sample−cpm spontaneous lysis)/(cpm maximal lysis−cpm spontaneous lysis) wherein cpm is counts per minute.

ADCC with reporter assay:
(a) plating about 5,000 Daudi cells in 10 μL in multi-well plates suitable for optical readings (e.g., 384-well OptiPlates from PerkinElmer Inc.) in a standard medium (e.g., RPMI 1640) supplemented with 25% low IgG serum;
(b) incubating each well for 6 hours at 37° C. with 10 μL engineered Jurkat cells stably expressing the FcγRIIIa receptor, V158 (high affinity) variant, and an NFAT response element driving expression of firefly luciferase as effector cells and 10 μL serially diluted CD38 antibody (0.0002-10 μg/mL);
(c) incubating each well 5 minutes at RT with 30 μL Luciferase substrate and measuring luminescence.

### Antibody-Dependent Cellular Phagocytosis (ADCP):

In one embodiment, the antibody variant according to any aspect or embodiment herein induces ADCP. In some embodiments, the antibody variants of the present invention may mediate ADCP when bound to CD38 on, for example, the surface of a CD38-expressing cell or cell membrane. The antibody variants of the present invention may mediate a higher ADCP when bound to CD38 on, for example, the surface of a CD38-expressing cell or cell membrane, than a control wherein the control is an isotype control antibody, e.g., such that the VH and VL sequences are those of antibody b12 as shown in Table 1.

Accordingly, in one embodiment, the antibody variant according to any aspect or embodiment disclosed herein, induces a higher ADCP against CD38-expressing target cells than a reference antibody, wherein the reference antibody differs from the antibody variant only in the one or more mutations in E430, E345 and/or S440 in the variant antibody. In an alternative embodiment, the reference antibody comprises the VH and VL region sequences of antibody b12, i.e., SEQ ID NO:12 and SEQ ID NO:16, respectively and CH and CL region sequences identical to the antibody variant.

In one specific embodiment, the CD38-expressing target cells are tumor cells, such as myeloma or lymphoma cells. Non-limiting examples of target cells that are tumor cells include those listed in Table 2.

Any in vitro or in vivo method or assay known by the skilled person and suitable for evaluating the ability of an antibody, such as an IgG antibody, to induce ADCP against CD38-expressing target cells can be used. Preferably, the assay comprises, in relevant part, the steps of the macrophage-based ADCP assay described in Example 5. In particular, the assay for determining the ADCP of CD38-expressing cells as mediated by a CD38 antibody may comprise the steps set out below: ADCP:
(a) differentiating freshly isolated monocytes to macrophages with 5 days incubation in GM-CSF-containing medium;
(b) plating about 100,000 macrophages per well in a multi-well plate in dendritic cell medium with GM-CSF;
(c) adding 20,000 CD38-antibody opsonized CD38-expressing cells (e.g., Daudi cells), labelled with a generic fluorescent membrane dye, per well for 45 minutes at 37° C.;
(d) measuring the percentage of CD14-positive, CD19-negative, membrane-dye-positive macrophages on a flow cytometer.

### Apoptosis:

The antibody variant for use according to the invention may, in one embodiment, not induce apoptosis in the absence of an Fc-cross-linking agent. In a further embodiment the antibody variant may induce apoptosis in the presence of an Fc-cross-linking agent but not in the absence of an Fc-cross-linking agent.

In one embodiment the Fc-cross-linking agent is an antibody.

In one embodiment apoptosis may be determined as described in Example 6.

### Trogocytosis:

In one embodiment, the antibody variant as disclosed herein induces trogocytosis, such as trogocytosis of CD38 from donor CD38-expressing cells to acceptor cells. Typical acceptor cells include T and B cells, monocytes/macrophages, dendritic cells, neutrophils, and NK cells. Preferably, the acceptor cells are lymphocytes expressing Fc-gamma- (Fcγ)-receptors, such as, e.g., macrophages or PBMCs. In particular, the antibody variants of the present invention may mediate an increased trogocytosis as compared to a control. The control can be, for example, a reference antibody with amino acid sequences (typically heavy- and light chain amino acid sequences) identical to the antibody variant except for the one or more mutations in E430, E345 and/or S440 in the variant antibody. In another embodiment, the control is a reference antibody with amino acid sequences (typically heavy- and light chain amino acid sequences) identical to the antibody variant except for different VH and VL sequences. For example, the control may be an isotype control antibody, e.g., such that the VH and VL sequences are those of antibody b12 as shown in Table 1.

Suitable assays for evaluating trogocytosis are known in the art and include, for example, the assay in Example 8. Non-limiting examples of assays for determining trogocytosis of CD38 expressing cells as mediated by a CD38 antibody include the following:

### Trogocytosis (Daudi Cells):

(a′) differentiating freshly isolated monocytes to macrophage with 5 days GM-CSF;
(b′) plating about 100,000 macrophages per well in dendritic cell medium with GM-CSF;
(c′) adding about 20,000 CD38 antibody-opsonized Daudi cells, labelled with a generic fluorescent membrane dye, per well for 45 minutes at 37° C.;
(d′) measuring CD38 expression on Daudi cells on a flow cytometer, wherein a reduction in CD38 on CD38-antibody opsonized Daudi cells as compared to a control indicates trogocytosis.

### Trogocytosis (Tregs):

(a) plating about 500,000 freshly isolated PBMCs per well in cell culture medium 0/N at 37° C.;
(b) adding about 100,000, CD38 antibody-opsonized Tregs, labelled with a generic fluorescent intracellular amine dye, per well overnight (0/N) at 37° C.; and
(c) measuring CD38 expression on Tregs on a flow cytometer, wherein a reduction in CD38 on CD38-antibody opsonized Tregs as compared to a control indicates trogocytosis.

In addition to Daudi cells (ATCC CCL-213), tumor cells suitable for the first assay include, without limitation, those listed in Table 2, particularly those with a high CD38 expression. Moreover, suitable CD38-expressing cells for the second assay include, in addition to Tregs, immune cells such as, e.g., NK cells, B cells, T cells and monocytes, as well as tumor cells listed in Table 2, particularly those with a low CD38 expression level.

Accordingly, in one embodiment, the antibody variant according to any aspect or embodiment disclosed herein induces a higher level of trogocytosis of a CD38-expressing target cell than a reference antibody, wherein the reference antibody comprises the VH and VL region sequences of antibody C, i.e., SEQ ID NO:1 and SEQ ID NO:5, respectively, and CH and CL region sequences identical to the antibody variant except for the one or more mutations in E430, E345 and/or S440.

In some embodiments, the antibody variant according to any aspect or embodiment disclosed herein induces a higher level of trogocytosis of CD38-expressing target cells than a reference antibody, wherein the reference antibody comprises the VH and VL region sequences of antibody B, i.e., SEQ ID NO:8 and SEQ ID NO:9, respectively and CH and CL region sequences identical to the antibody variant.

In some embodiments, the antibody variant according to any aspect or embodiment disclosed herein induces a higher level trogocytosis of CD38-expressing target cells than a reference antibody, wherein the reference antibody comprises the VH and VL region sequences of antibody A, i.e., SEQ ID NO:10 and SEQ ID NO:11, respectively and CH and CL region sequences identical to the antibody variant.

In some embodiments, the antibody variant according to any aspect or embodiment disclosed herein induces a higher level trogocytosis of CD38-expressing target cells than a reference antibody, wherein the reference antibody comprises the VH and VL region sequences of antibody b12, i.e., SEQ ID NO:12 and SEQ ID NO:16, respectively and CH and CL region sequences identical to the antibody variant.

### Modulation of CD38 Enzyme Activity

The antibody variant according to any aspect or embodiment herein can typically modulate one or more enzyme activities of human CD38. In one embodiment, the antibody variant as disclosed herein has an inhibitory effect on CD38 cyclase activity, e.g. as compared to a control, e.g., an isotype control antibody such as antibody b12. For example, the antibody variant may have an inhibitory effect on the cyclase activity of CD38 expressed by a cell, such as a tumor cell, and/or an inhibitory effect on isolated CD38, such as a soluble fragment of CD38 (e.g., SEQ ID NO:39).

Any in vitro or in vivo method or assay known by the skilled person and suitable for evaluating the ability of an anti-CD38 antibody to inhibit CD38 cyclase activity can be used. Suitable assays for testing CD38 cyclase activity are, for example, described in WO 2006/099875 A1 and WO 2011/154453 A1. Preferably, the method comprises, in relevant part, the steps of the particular assay described in Example 6, testing for cyclase activity using nicotinamide guanine dinucleotide sodium salt (NGD) as a substrate for CD38. NGD, which is non-fluorescent, is cyclized by CD38 to a fluorescent analog of cADPR, cyclic GDP-ribose (see, e.g., Comb, Chem High Throughput Screen. 2003 June; 6(4):367-79A). A non-limiting example of an assay comprises the following steps for determining the inhibition of CD38 cyclase activity:
(a) seeding 200,000 Daudi or Wien133 cells in 100 μL 20 mM Tris-HCL per well; or seeding 0.6 μg/mL His-tagged soluble CD38 (SEQ ID NO:39) in 100 μL 20 mM Tris-HCL per well in a multi-well plate;
(b) adding 1 μg/mL CD38 antibody and 80 μM NGD to each well;
(c) measuring fluorescence until a plateau is reached (e.g.; 5, 10 or 30 minutes); and
(d) determining the percentage inhibition as compared to a control, such as a well incubated with an isotype control antibody.

In one embodiment, in such an assay, an antibody variant is capable of inhibiting the cyclase activity of CD38, specifically the maximum percent of NGD conversion, with at least about 40%, such as at least about 50%, such as at least about 60%, such as between about 40% to about 60%, as compared to a control, typically CD38 cyclase activity in the presence of an isotype control antibody. For example, the isotype control antibody may comprise the VH and VL region sequences of antibody b12, i.e., SEQ ID NO:12 and SEQ ID NO:16, respectively, and CH and CL region sequences identical to the antibody variant. In a specific embodiment, the assay utilizes hisCD38 (SEQ ID NO:39) for determining the cyclase activity.

In some embodiments, the antibody variant according to any aspect or embodiment disclosed herein has an increased (i.e., more effective) inhibition of CD38 cyclase activity as compared to a reference antibody, wherein the reference antibody comprises the VH and VL region sequences of antibody B, i.e., SEQ ID NO:8 and SEQ ID NO:9, respectively and CH and CL region sequences identical to the antibody variant.

In some embodiments, the antibody variant according to any aspect or embodiment disclosed herein has an increased (i.e., more effective) inhibition of CD38 cyclase activity as compared to a reference antibody, wherein the reference antibody comprises the VH and VL region sequences of antibody A, i.e., SEQ ID NO:10 and SEQ ID NO:11, respectively and CH and CL region sequences identical to the antibody variant.

Moreover, in some embodiments, an antibody variant as described herein induces apoptosis of CD38-expressing cells in the presence, but not in the absence, of Fc-crosslinking antibodies. These functionalities can both be measured in an assay comprising, in relevant part, the steps of the apoptosis assay described in Example 6. In one embodiment, an apoptosis assay may comprise the steps of:
(a) plating 100,000 CD38-expressing tumor cells in 100 μL culture medium supplemented with 0.2% BSA per well;
(b) incubating each well 0/N at 37° C. with serially diluted CD38 antibody (0.0002-10 μg/mL) and 10 μg/mL goat-anti-human IgG1;
(c) measuring the percentage of dead cells on a flow cytometer.

### Conjugates

In one aspect, the present invention relates to an antibody variant which is conjugated to a drug, cytotoxic agent, toxin, radiolabel or radioisotope.

In one embodiment, antibody variants comprising one or more radiolabeled amino acids are provided. A radiolabeled variant may be used for in vitro diagnostic purposes, in vivo diagnostic purposes, therapeutic purposes or a combination thereof. Non-limiting examples of radiolabels for antibodies include ³H, ¹⁴C, ¹⁵N, ³⁵S, ⁹⁰Y, ⁹⁹Tc, ¹²⁵I, ¹³¹I, and ¹⁸⁶Re. Methods for preparing radiolabeled amino acids and related peptide derivatives are known in the art, (see, for instance Junghans et al., in Cancer Chemotherapy and Biotherapy 655-686 (2n° Ed., Chafner and Longo, eds., Lippincott Raven (1996)) and U.S. Pat. Nos. 4,681,581, 4,735,210, 5,101,827, U.S. Pat. No. 5,102,990 (U.S. RE35,500), U.S. Pat. Nos. 5,648,471 and 5,697,902. For example, a radioisotope of a halogen such as iodine or bromine may be conjugated by the chloramine-T method.

In one embodiment, the antibody variant of the present invention is conjugated to a radioisotope or to a radioisotope-containing chelate. For example, the variant can be conjugated to a chelator linker, e.g. DOTA, DTPA or tiuxetan, which allows for the antibody to be complexed with a radioisotope. The variant may also or alternatively comprise or be conjugated to one or more radiolabeled amino acids or other radiolabeled molecule. A radiolabeled variant may be used for both diagnostic and therapeutic purposes. In one embodiment the variant of the present invention is conjugated to an alpha-emitter. Non-limiting examples of alpha-emitting radioisotopes include ²¹³Bs, ²²⁵Ac and ²²⁷Th.

In one embodiment, the antibody variant is attached to a chelator linker, e.g. tiuxetan, which allows for the antibody variant to be conjugated to a radioisotope.

### Nucleic Acids

Antibodies are well known as therapeutics which may be used in treatment of various diseases. Another method for administration of an antibody to a subject in need thereof includes administration of a nucleic acid or a combination of nucleic acids encoding said antibody for in vivo expression of said antibody.

Hence in one aspect, the present invention also relates to a nucleic acid encoding the heavy chain of an antibody variant according to the present invention, wherein said heavy chain comprises a VH region comprising a VH CDR1 having the sequence as set forth in SEQ ID NO:2, a VH CDR2 having the sequence as set forth in SEQ ID NO:3, a VH CDR3 having the sequence as set forth in SEQ ID NO:4 and a human IgG1 CH region with a mutation in one or more of E430, E345 and S440, the amino acid residues being numbered according to the EU index.

In one aspect the present invention also relates to a nucleic acid or a combination of nucleic acids, encoding an antibody variant according to the present invention.

In some embodiments the present invention relates to a nucleic acid or a combination of nucleic acids encoding an antibody variant comprising:
a) an antigen-binding region comprising a VH CDR1 having the sequence as set forth in SEQ ID NO:2, a VH CDR2 having the sequence as set forth in SEQ ID NO:3, a VH CDR3 having the sequence as set forth in SEQ ID NO:4, a VL CDR1 having the sequence as set forth in SEQ ID NO:6, a VL CDR2 having the sequence AAS, and a VL CDR3 having the sequence as set forth in SEQ ID NO:7, and
b) a variant Fc region comprising a mutation in one or more amino acid residues selected from the group corresponding to E430, E345 and S440 in a human IgG1 heavy chain, wherein the amino acid residues are numbered according to the EU index.

In one embodiment, the antibody variant of the present invention is encoded by one nucleic acid. Thus, the nucleotide sequences encoding the antibody variant of the present invention are present in one nucleic acid or the same nucleic acid molecule.

In another embodiment the antibody variant of the present invention is encoded by a combination of nucleic acids, typically by two nucleic acids. In one embodiment said combination of nucleic acids comprise a nucleic acid encoding the heavy chain of said antibody variant and a nucleic acid encoding the light chain of said antibody variant.

In some embodiments the present invention relates to a nucleic acid or a combination of nucleic acids encoding an antibody variant comprising:
a) a heavy chain comprising a VH region comprising a VH CDR1 having the sequence as set forth in SEQ ID NO:2, a VH CDR2 having the sequence as set forth in SEQ ID NO:3, a VH CDR3 having the sequence as set forth in SEQ ID NO:4 and a human IgG1 CH region with a mutation in one or more of E430, E345 and S440, the amino acid residues being numbered according to the EU index;
b) a light chain comprising a VL region comprising a VL CDR1 having the sequence as set forth in SEQ ID NO:6, a VL CDR2 having the sequence AAS, and a VL CDR3 having the sequence as set forth in SEQ ID NO:7.

In one embodiment, the antibody variant of the present invention is encoded by one nucleic acid. Thus, the nucleotide sequences encoding the antibody variant of the present invention are present in one nucleic acid or the same nucleic acid molecule.

In another embodiment the antibody variant of the present invention is encoded by a combination of nucleic acids, typically by two nucleic acids. In one embodiment said combination of nucleic acids comprise a nucleic acid encoding the heavy chain of said antibody variant and a nucleic acid encoding the light chain of said antibody variant.

As described above the nucleic acids may be used as a mean for supplying therapeutic proteins, such as antibodies, to a subject in need thereof.

In some embodiments, said nucleic acid may be deoxyribonucleic acid (DNA). DNAs and methods of preparing DNA suitable for in vivo expression of therapeutic proteins, such as antibodies are well known to a person skilled in the art, and include but is not limited to that described by Patel A et al., 2018, Cell Reports 25, 1982-1993.

In some embodiments, said nucleic acid may be ribonucleic acid (RNA), such as messenger RNA (mRNA). In some embodiments, the mRNA may comprise only naturally occurring nucleotides. In some embodiments the mRNA may comprise modified nucleotides, wherein modified refers to said nucleotides being chemically different from the naturally occurring nucleotides. In some embodiments the mRNA may comprise both naturally occurring and modified nucleotides.

Different nucleic acids suitable for in vivo expression of therapeutic proteins, such as antibodies, in a subject are well known to a person skilled in the art. For example, a mRNA suitable for expression a therapeutic antibody in a subject, often comprise an Open Reading Frame (ORF), flanked by Untranslated Regions (UTRs) comprising specific sequences, and 5′ and 3′ ends being formed by a cap structure and a poly(A)tail (see e.g. Schlake et al., 2019, Molecular Therapy Vol. 27 No 4 April).

Examples of methods for optimization of RNA and RNA molecules suitable, e.g. mRNA, for in vivo expression include, but are not limited to those described in U.S. Pat. Nos. 9,254,311; 9,221,891; US20160185840 and EP3118224.

Naked nucleic acid(s) which are administered to a subject for in vivo expression are prone to degradation and/or of causing an immunogenic response in the subject. Furthermore, for in vivo expression of the antibody encoded by the nucleic acid said nucleic acid typically is administered in a form suitable for the nucleic acid to enter the cells of the subject. Different methods for delivering a nucleic acid for in vivo expression exist and include both methods involving mechanical and chemical means. For example, such methods may involve electroporation or tattooing the nucleic acid onto the skin (Patel et al., 2018, Cell Reports 25, 1982-1993). Other methods suitable for administration of the nucleic acid to a subject involve administration of the nucleic acid in a suitable formulation. Thus the present invention also relates to a delivery vehicle comprising a nucleic acid of the present invention.

In some embodiments, said delivery vehicle may comprise a nucleic acid encoding a heavy chain of an antibody variant according to the present invention. Thus in one embodiment said nucleic acid may encode a heavy chain comprising a VH region comprising a VH CDR1 having the sequence as set forth in SEQ ID NO:2, a VH CDR2 having the sequence as set forth in SEQ ID NO:3, a VH CDR3 having the sequence as set forth in SEQ ID NO:4 and a human IgG1 CH region with a mutation in one or more of E430, E345 and S440, the amino acid residues being numbered according to the EU index.

In some embodiments, the present invention also relates to a delivery vehicle comprising a nucleic acid encoding a light chain of an antibody variant according to the present invention. Thus in one embodiment said nucleic acid may encode a light chain comprising a VL region comprising a VL CDR1 having the sequence as set forth in SEQ ID NO:6, a VL CDR2 having the sequence AAS, and a VL CDR3 having the sequence as set forth in SEQ ID NO:7.

The present invention also relates to a mixture of delivery vehicles comprising a delivery vehicle comprising a nucleic acid encoding a heavy chain of an antibody variant according to the present invention and delivery vehicle comprising a nucleic acid encoding a light chain of an antibody variant according to the present invention. Thus in one embodiment said mixture of delivery vehicles comprise a delivery vehicle comprising a nucleic acid encoding a heavy chain comprising a VH region comprising a VH CDR1 having the sequence as set forth in SEQ ID NO:2, a VH CDR2 having the sequence as set forth in SEQ ID NO:3, a VH CDR3 having the sequence as set forth in SEQ ID NO:4 and a human IgG1 CH region with a mutation in one or more of E430, E345 and S440, the amino acid residues being numbered according to the EU index; and a delivery vehicle comprising a nucleic acid encoding a light chain comprising a VL region comprising a VL CDR1 having the sequence as set forth in SEQ ID NO:6, a VL CDR2 having the sequence AAS, and a VL CDR3 having the sequence as set forth in SEQ ID NO:7.

In some embodiments, said delivery vehicle comprises a nucleic acid or a combination of nucleic acids encoding the heavy and a nucleic light chain of an antibody variant according to the present invention.

Thus in one embodiment said delivery vehicle may comprise a nucleic acid encoding a heavy chain comprising a VH region comprising a VH CDR1 having the sequence as set forth in SEQ ID NO:2, a VH CDR2 having the sequence as set forth in SEQ ID NO:3, a VH CDR3 having the sequence as set forth in SEQ ID NO:4 and a human IgG1 CH region with a mutation in one or more of E430, E345 and S440, the amino acid residues being numbered according to the EU index; and a light chain comprising a VL region comprising a VL CDR1 having the sequence as set forth in SEQ ID NO:6, a VL CDR2 having the sequence AAS, and a VL CDR3 having the sequence as set forth in SEQ ID NO:7. Thus, the nucleic acid sequences encoding the heavy and light chain of the antibody variant according to the present invention are present in one (the same) nucleic acid molecule.

In another embodiment said delivery vehicle may comprise a nucleic acid encoding a heavy chain comprising a VH region comprising a VH CDR1 having the sequence as set forth in SEQ ID NO:2, a VH CDR2 having the sequence as set forth in SEQ ID NO:3, a VH CDR3 having the sequence as set forth in SEQ ID NO:4 and a human IgG1 CH region with a mutation in one or more of E430, E345 and S440, the amino acid residues being numbered according to the EU index; and a nucleic acid encoding a light chain comprising a VL region comprising a VL CDR1 having the sequence as set forth in SEQ ID NO:6, a VL CDR2 having the sequence AAS, and a VL CDR3 having the sequence as set forth in SEQ ID NO:7. Thus, the nucleic acid sequences encoding the heavy and light chain of the antibody variant according to the present invention are present on separate or different nucleic acid molecules.

In some embodiments said delivery vehicle may be a lipid formulation. The lipids of the formulation may particle(s), such as a lipid nanoparticle(s) (LNPs). The nucleic acid or combination of nucleic acids of the present may be encapsulated within said particle, e.g. within said LNP.

Different lipid formulations suitable for administration of a nucleic acid to a subject for in vivo expression are well known to a person skilled in the art. For example, said lipid formulation may typically comprise lipids, ionizable aminolipids, PEG-lipids, cholesterol or any combination thereof.

Various forms and methods for preparation of lipid formulations suitable for administration of a nucleic acid to a subject for expression of a therapeutic antibody are well known in the art. Examples of such lipid formulations include but are not limited to those described in US20180170866 (Arcturus), EP 2391343 (Arbutus), WO 2018/006052 (Protiva), WO2014152774 (Shire Human Genetics), EP 2 972 360 (Translate Bio), U.S. Ser. No. 10/195,156 (Moderna) and US20190022247 (Acuitas).

### Production of Variant Antibody

In another aspect, the present invention also relates to a method of increasing at least one effector function of an antibody comprising CDR, VH and/or VL amino acid sequences of antibody C, comprising introducing a mutation into the antibody in one or more amino acid residue(s) corresponding to E430, E345, and S440 in the Fc region of a human IgG1 heavy chain, numbered according to the EU-index.

So, in certain embodiments, there is provided a method of increasing an effector function of a parent antibody comprising an Fc region and an antigen-binding region binding to CD38, which method comprises introducing into the Fc region a mutation in one or more amino acid residues selected from the group corresponding to E430, E345, and S440 in the Fc region of a human IgG1 heavy chain, wherein the amino acid residues are numbered according to the EU index; and
wherein the antigen-binding region comprises a VH CDR1 having the sequence as set forth in SEQ ID NO:2, a VH CDR2 having the sequence as set forth in SEQ ID NO:3, a VH CDR3 having the sequence as set forth in SEQ ID NO:4, a VL CDR1 having the sequence as set forth in SEQ ID NO:6, a VL CDR2 having the sequence AAS, and a VL CDR3 having the sequence as set forth in SEQ ID NO:7.

In other certain embodiments, there is provided a method of producing a variant of a parent antibody comprising an Fc region and an antigen-binding region, optionally the variant having an increased effector function as compared to the parent antibody, which method comprises
(a) introducing into the Fc region a mutation in one or more amino acid residues selected from the group corresponding to E430, E345, and S440 in the Fc region of a human IgG1 heavy chain to obtain a variant antibody,
(b) selecting any variant antibody having an increased effector function as compared to the parent antibody, and
(c) producing said variant antibody in a recombinant host cell,
wherein the antigen-binding region comprises VH CDR1 having the sequence as set forth in SEQ ID NO:2, a VH CDR2 having the sequence as set forth in SEQ ID NO:3, a VH CDR3 having the sequence as set forth in SEQ ID NO:4, a VL CDR1 having the sequence as set forth in SEQ ID NO:6, a VL CDR2 having the sequence AAS, and a VL CDR3 having the sequence as set forth in SEQ ID NO:7.

In one embodiment of any one of the aforementioned methods, the effector function is CDC.

In one embodiment of any one of the aforementioned methods, the effector function is trogocytosis.

In one embodiment of any one of the aforementioned methods, the effector function is CDC and trogocytosis.

In one embodiment of any of the aforementioned methods, the mutation in the one or more amino acid residues is selected from the group corresponding to E430G, E430S, E430F, E430T, E345K, E345Q, E345R, E345Y, S440Y and S440W. For example, the mutation in the one or more amino acid residue(s) may comprise or consist of E430G.

In one embodiment of any of the aforementioned methods, the Fc region of the parent antibody is, apart from the recited mutation(s), a human IgG1, IgG2, IgG3 or IgG4 Fc region, or an isotype mixture thereof. Optionally comprising an Fc region of one of the sequences set forth as SEQ ID NO:19, SEQ ID NO:20, SEQ ID NO:21, SEQ ID NO:22, SEQ ID NO:23, SEQ ID NO:34, SEQ ID NO:35, SEQ ID NO:45 and SEQ ID NO:36. In a particular embodiment, the Fc region of the parent antibody is a human IgG1 Fc region. For example, the parent antibody can be a human full-length IgG1 antibody, optionally a human monoclonal full-length bivalent IgG1,κ antibody. Additionally, the parent antibody can be a monospecific or bispecific antibody, such as a monospecific antibody.

While the Fc region of the parent antibody is typically a naturally-occurring (wild-type) sequence, in some embodiments, the Fc region of the parent antibody comprises one or more further mutations, as described elsewhere herein.

The present invention also relates to an antibody obtained or obtainable according to any of the above described methods.

The invention also provides isolated nucleic acids and vectors encoding an antibody variant according to any one of the aspects and embodiments described herein, as well as vectors and expression systems encoding the variants. Suitable nucleic acid constructs, vectors and expression systems for antibodies and variants thereof are known in the art, and include, but are not limited to, those described in the Examples. In embodiments where the variant antibody comprises HC and LC that are separate polypeptides rather than contained in a single polypeptide (e.g., as in a scFv-Fc fusion protein), the nucleotide sequences encoding the heavy and light chains may be present in the same or different nucleic acids or vectors.

In one aspect, the invention relates to a nucleic acid or an expression vector comprising
(i) a nucleotide sequence encoding a heavy chain sequence of an antibody variant according to any one of the embodiments disclosed herein;
(ii) a nucleotide sequence encoding a light chain sequence of an antibody variant according to any one of the embodiments disclosed herein; or
(iii) both (i) and (ii).

In one aspect, the invention relates to a nucleic acid or an expression vector comprising a nucleotide sequence encoding a heavy chain sequence of an antibody variant according to any one of the embodiments disclosed herein.

In one aspect, the invention relates to a nucleic acid sequence or an expression vector comprising a nucleotide sequence encoding a heavy chain sequence and a light chain sequence of an antibody variant according to any one of the embodiments disclosed herein.

In one aspect, the invention relates to a combination of a first and a second nucleic acid or a combination of a first and second expression vector, optionally in the same host cell, where the first comprises a nucleotide sequence according to (i), and the second comprises a nucleotide sequence according to (ii).

An expression vector in the context of the present invention may be any suitable vector, including chromosomal, non-chromosomal, and synthetic nucleic acid vectors (a nucleic acid sequence comprising a suitable set of expression control elements). Examples of such vectors include derivatives of SV40, bacterial plasmids, phage DNA, baculovirus, yeast plasmids, vectors derived from combinations of plasmids and phage DNA, and viral nucleic acid (RNA or DNA) vectors. In one embodiment, a nucleic acid is comprised in a naked DNA or RNA vector, including, for example, a linear expression element (as described in for instance Sykes and Johnston, Nat Biotech 17, 355 59 (1997)), a compacted nucleic acid vector (as described in for instance U.S. Pat. No. 6,077,835 and/or WO 00/70087), a plasmid vector such as pBR322, pUC 19/18, or pUC 118/119, a “midge” minimally-sized nucleic acid vector (as described in for instance Schakowski et al., Mol Ther 3, 793 800 (2001)), or as a precipitated nucleic acid vector construct, such as a CaP04-precipitated construct (as described in for instance WO200046147, Benvenisty and Reshef, PNAS USA 83, 9551 55 (1986), Wigler et al., Cell 14, 725 (1978), and Coraro and Pearson, Somatic Cell Genetics 7, 603 (1981)). Such nucleic acid vectors and the usage thereof are well known in the art (see for instance U.S. Pat. Nos. 5,589,466 and 5,973,972).

In one embodiment, the vector is suitable for expression of the antibody variant in a bacterial cell. Examples of such vectors include expression vectors such as BlueScript (Stratagene), pIN vectors (Van Heeke & Schuster, J Biol Chem 264, 5503 5509 (1989), pET vectors (Novagen, Madison Wis.) and the like).

An expression vector may also or alternatively be a vector suitable for expression in a yeast system. Any vector suitable for expression in a yeast system may be employed. Suitable vectors include, for example, vectors comprising constitutive or inducible promoters such as alpha factor, alcohol oxidase and PGH (reviewed in: F. Ausubel et al., ed. Current Protocols in Molecular Biology, Greene Publishing and Wiley InterScience New York (1987), and Grant et al., Methods in Enzymol 153, 516 544 (1987)).

An expression vector may also or alternatively be a vector suitable for expression in mammalian cells, e.g. a vector comprising glutamine synthetase as a selectable marker, such as the vectors described in Bebbington (1992) Biotechnology (NY) 10:169-175.

A nucleic acid and/or vector may also comprises a nucleic acid sequence encoding a secretion/localization sequence, which can target a polypeptide, such as a nascent polypeptide chain, to the periplasmic space or into cell culture media. Such sequences are known in the art, and include secretion leader or signal peptides.

The expression vector may comprise or be associated with any suitable promoter, enhancer, and other expression-facilitating elements. Examples of such elements include strong expression promoters (e. g., human CMV IE promoter/enhancer as well as RSV, SV40, SL3 3, MMTV, and HIV LTR promoters), effective poly (A) termination sequences, an origin of replication for plasmid product in E. coli, an antibiotic resistance gene as selectable marker, and/or a convenient cloning site (e.g., a polylinker). Nucleic acids may also comprise an inducible promoter as opposed to a constitutive promoter such as CMV IE.

In one embodiment, the antibody variant-encoding expression vector may be positioned in and/or delivered to the host cell or host animal via a viral vector.

The invention also provides a recombinant host cell which produces an antibody variant as disclosed herein, optionally wherein the host cell comprises the isolated nucleic acid(s) or vector(s) according to the present invention. Typically, the host cell has been transformed or transfected with the nucleic acid(s) or vector(s). The recombinant host cell of claim can be, for example, a eukaryotic cell, a prokaryotic cell, or a microbial cell, e.g., a transfectoma. In a particular embodiment the host cell is a eukaryotic cell. In a particular embodiment the host cell is a prokaryotic cell. In some embodiments, the antibody is a heavy-chain antibody. In most embodiments, however, the antibody variant will contain both a heavy and a light chain and thus said host cell expresses both heavy- and light-chain-encoding construct, either on the same or a different vector.

Examples of host cells include yeast, bacterial, plant and mammalian cells, such as CHO, CHO-S, HEK, HEK293, HEK-293F, Expi293F, PER.C6, NS0 cells, Sp2/0 cells or lymphocytic cells. In one embodiment the host cell is a CHO (Chinese Hamster Ovary) cell. For example, in one embodiment, the host cell may comprise a first and second nucleic acid construct stably integrated into the cellular genome, wherein the first encodes the heavy chain and the second encodes the light chain of an antibody variant as disclosed herein. In another embodiment, the present invention provides a cell comprising a non-integrated nucleic acid, such as a plasmid, cosmid, phagemid, or linear expression element, which comprises a first and second nucleic acid construct as specified above.

In one embodiment, said host cell is a cell which is capable of Asn-linked glycosylation of proteins, e.g. a eukaryotic cell, such as a mammalian cell, e.g. a human cell. In a further embodiment, said host cell is a non-human cell which is genetically engineered to produce glycoproteins having human-like or human glycosylation. Examples of such cells are genetically-modified Pichia pastoris (Hamilton et al., Science 301 (2003) 1244-1246; Potgieter et al., J. Biotechnology 139 (2009) 318-325) and genetically-modified Lemna minor (Cox et al., Nature Biotechnology 12 (2006) 1591-1597).

In one embodiment, said host cell is a host cell which is not capable of efficiently removing C-terminal lysine K447 residues from antibody heavy chains. For example, Table 2 in Liu et al. (2008) J Pharm Sci 97: 2426 (incorporated herein by reference) lists a number of such antibody production systems, e.g. Sp2/0, NS/0 or transgenic mammary gland (goat), wherein only partial removal of C-terminal lysines is obtained. In one embodiment, the host cell is a host cell with altered glycosylation machinery. Such cells have been described in the art and can be used as host cells in which to express variants of the invention to thereby produce an antibody with altered glycosylation. See, for example, Shields, R. L. et al. (2002) J. Biol. Chem. 277:26733-26740; Umana et al. (1999) Nat. Biotech. 17:176-1, as well as EP1176195; WO03/035835; and WO99/54342. Additional methods for generating engineered glycoforms are known in the art, and include but are not limited to those described in Davies et al., 2001, Biotechnol Bioeng 74:288-294; Shields et al, 2002, J Biol Chem 277:26733-26740; Shinkawa et al., 2003, J Biol Chem 278:3466-3473), U.S. Pat. No. 6,602,684, WO00/61739A1; WO01/292246A1; WO02/311140A1; WO 02/30954A1; Potelligent™ technology (Biowa, Inc. Princeton, N.J.); GlycoMAb™ glycosylation engineering technology (GLYCART biotechnology AG, Zurich, Switzerland); US 20030115614; Okazaki et al., 2004, JMB, 336: 1239-49, as well as those described in WO2018/114877 WO2018/114878 and WO2018/114879.

In an even further aspect, the invention relates to a transgenic non-human animal or plant comprising nucleic acids encoding one or two sets of a human heavy chain and a human light chain, wherein the animal or plant produces an antibody variant as disclosed herein.

In one embodiment, there is provided a method of producing an antibody variant as disclosed herein, comprising cultivating the recombinant host cell in a culture medium and under conditions suitable for producing the antibody variant and, optionally, purifying or isolating the antibody variant from the culture medium.

In one embodiment, there is provided an antibody obtained or obtainable by the method described above.

### Compositions and Kit-of-Parts

The present invention also relates to a composition comprising an antibody variant according to the present invention, a nucleic acid according to the present invention, an expression vector according to the present invention or a host cell according to the present invention.

In a further embodiment the composition according to the present invention is a pharmaceutical composition, typically comprising a pharmaceutically acceptable carrier. In one embodiment the pharmaceutical composition contains an antibody variant as defined in any aspect or embodiment disclosed herein, or an expression vector as defined in any aspect or embodiment disclosed herein.

In yet a further embodiment, the invention relates to a pharmaceutical composition comprising:
an antibody variant as defined in any of the aspects and embodiments disclosed herein, and
a pharmaceutically acceptable carrier.

The pharmaceutical compositions may be formulated in accordance with conventional techniques such as those disclosed in Remington: The Science and Practice of Pharmacy, 19th Edition, Gennaro, Ed., Mack Publishing Co., Easton, Pa., 1995. A pharmaceutical composition of the present invention may e.g. include diluents, fillers, salts, buffers, detergents (e.g., a nonionic detergent, such as Tween-20 or Tween-80), stabilizers (e. g., sugars or protein-free amino acids), preservatives, tissue fixatives, solubilizers, and/or other materials suitable for inclusion in a pharmaceutical composition.

Pharmaceutically acceptable carriers include any and all suitable solvents, dispersion media, coatings, antibacterial and antifungal agents, isotonicity agents, antioxidants and absorption delaying agents, and the like that are physiologically compatible with an antibody variant of the present invention. Examples of suitable aqueous and nonaqueous carriers which may be employed in the pharmaceutical compositions of the present invention include water, saline, phosphate buffered saline, ethanol, dextrose, polyols (such as glycerol, propylene glycol, polyethylene glycol, and the like), and suitable mixtures thereof, vegetable oils, carboxymethyl cellulose colloidal solutions, tragacanth gum and injectable organic esters, such as ethyl oleate, and/or various buffers. Pharmaceutically acceptable carriers include sterile aqueous solutions or dispersions and sterile powders for the extemporaneous preparation of sterile injectable solutions or dispersion. Proper fluidity may be maintained, for example, by the use of coating materials, such as lecithin, by the maintenance of the required particle size in the case of dispersions, and by the use of surfactants.

The pharmaceutical compositions may also comprise pharmaceutically acceptable antioxidants for instance (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole, butylated hydroxytoluene, lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The pharmaceutical compositions may also comprise isotonicity agents, such as sugars, polyalcohols, such as mannitol, sorbitol, glycerol or sodium chloride in the compositions.

The pharmaceutical compositions may also contain one or more adjuvants appropriate for the chosen route of administration such as preservatives, wetting agents, emulsifying agents, dispersing agents, preservatives or buffers, which may enhance the shelf life or effectiveness of the pharmaceutical composition. The pharmaceutical composition of the present invention may be prepared with carriers that will protect the antibody against rapid release, such as a controlled release formulation, including implants, transdermal patches, and microencapsulated delivery systems. Such carriers may include gelatin, glyceryl monostearate, glyceryl distearate, biodegradable, biocompatible polymers such as ethylene vinyl acetate, polyanhydrides, polyglycolic acid, collagen, polyorthoesters, and polylactic acid alone or with a wax, or other materials well known in the art. Methods for the preparation of such formulations are generally known to those skilled in the art.

Sterile injectable solutions may be prepared by incorporating the active compound in the required amount in an appropriate solvent with one or a combination of ingredients e.g. as enumerated above, as required, followed by sterilization microfiltration. Generally, dispersions are prepared by incorporating the active compound into a sterile vehicle that contains a basic dispersion medium and the required other ingredients e.g. from those enumerated above. In the case of sterile powders for the preparation of sterile injectable solutions, examples of methods of preparation are vacuum drying and freeze-drying (lyophilization) that yield a powder of the active ingredient plus any additional desired ingredient from a previously sterile-filtered solution thereof.

The actual dosage levels of the active ingredients in the pharmaceutical compositions may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

The pharmaceutical composition may be administered by any suitable route and mode. In one embodiment, a pharmaceutical composition of the present invention is administered parenterally. “Administered parenterally” as used herein means modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion.

In one embodiment the pharmaceutical composition is administered by intravenous or subcutaneous injection or infusion.

The invention also relates to kit-of-parts for simultaneous, separate or sequential use in therapy comprising an antibody variant according to the invention, or a composition comprising an antibody variant according to the invention, optionally wherein the kit-of-parts contains more than one dosage of the antibody variant.

In one embodiment, the kit-of-parts comprises such as antibody variant or composition in one or more containers such as vials.

In one embodiment, the kit-of-parts comprises such as antibody variant or composition for simultaneous, separate or sequential use in therapy.

### Therapeutic Applications

The antibody variants of the present invention have numerous therapeutic utilities involving the treatment of diseases and disorders involving cells expressing CD38, e.g., tumor cells or immune cells expressing CD38. For example, the antibody variants may be administered to cells in culture, e.g., in vitro or ex vivo, or to human subjects, e.g., in vivo, to treat or prevent a variety of disorders and diseases. As used herein, the term “subject” is intended to include human and non-human animals which may benefit or respond to the antibody. Subjects may for instance include human patients having diseases or disorders that may be corrected or ameliorated by modulating CD38 function, such as enzymatic activity, and/or induction of lysis and/or eliminating/reducing the number of CD38 expressing cells and/or reducing the amount of CD38 on the cell membrane. Accordingly, the antibody variants may be used to elicit in vivo or in vitro one or more of the following biological activities: CDC of a cell expressing CD38 in the presence of complement; inhibition of CD38 cyclase activity; phagocytosis or ADCC of a cell expressing CD38 in the presence of human effector cells; and trogocytosis of CD38-expressing cells, such as tumor cells or immune cells.

Thus, in one aspect, the present invention relates to the antibody variant according to the present invention, the nucleic acid or combination of nucleic acids according to the present invention, the delivery vehicle according to the present invention, the expression vector according to the present invention, the host cell according to the present invention, the composition according to the present invention, or the pharmaceutical composition according to the present invention for use as a medicament.

In one aspect, the present invention relates to the use of the antibody variant according to the present invention, the nucleic acid or combination of nucleic acids according to the present invention, the delivery vehicle according to the present invention, the expression vector according to the present invention, the host cell according to the present invention, the composition according to the present invention, or the pharmaceutical composition according to the present invention in the preparation of a medicament for treating or preventing a disease or disorder.

In one aspect, the present invention relates to the antibody variant according to the present invention, the nucleic acid or combination of nucleic acids according to the present invention, the delivery vehicle according to the present invention, the expression vector according to the present invention, the host cell according to the present invention, the composition according to the present invention, or the pharmaceutical composition according to the present invention for use in the treatment or prevention of a disease or disorder, such as for use in the treatment or prevention of a disease or disorder involving cells expressing CD38, e.g. for use in treating a disease involving cells expressing CD38. In one aspect, the present invention relates to the antibody variant according to the present invention, the nucleic acid according to the present invention, the expression vector according to the present invention, the host cell according to the present invention, the composition according to the present invention, or the pharmaceutical composition according to the present invention for use in inducing a CDC-response against a tumor comprising cells expressing CD38.

In one aspect, the present invention relates to a method of treatment of a disease or disorder comprising administering the antibody variant according to the present invention, the nucleic acid or combination of nucleic acids according to the present invention, the delivery vehicle according to the present invention, the expression vector according to the present invention, the host cell according to claim the present invention, the composition according to the present invention, or the pharmaceutical composition according to the present invention to a subject in need thereof.

In one aspect, the invention relates to the antibody variant according to any aspect or embodiment for use as a medicament.

In one aspect, the invention relates to the use of the antibody variant according to any aspect or embodiment in the preparation of a medicament for treating or preventing a disease or disorder.

In one aspect, the invention relates to the antibody variant according to any aspect or embodiment for use in the treatment or prevention of a disease or disorder.

In one aspect, the invention relates to a method of treating a disease or disorder, comprising administering the antibody variant according to any aspect or embodiment to a subject in need thereof, typically in a therapeutically effective amount and/or for a time sufficient to treat the disease or disorder.

In one aspect, the invention relates to a pharmaceutical composition comprising the antibody variant according to any aspect or embodiment, for use as a medicament.

In one aspect, the invention relates to a pharmaceutical composition comprising the antibody variant according to any aspect or embodiment for use in the treatment or prevention of a disease or disorder.

In one aspect, the invention relates to a method of treatment of a disease or disorder comprising administering a pharmaceutical composition comprising the antibody variant according to any aspect or embodiment to a subject in need thereof, typically in a therapeutically effective amount and/or for a time sufficient to treat the disease or disorder.

In one aspect, the present invention relates to a method of treating a disease or disorder, comprising the steps of
selecting a subject suffering from the disease or disorder, and
administering to the subject the antibody variant according to any aspect or embodiment, or a pharmaceutical composition comprising the antibody variant, typically in a therapeutically effective amount and/or for a time sufficient to treat the disease or disorder.

In one embodiment, the disease or disorder involving cells expressing CD38 is cancer, i.e. a tumorigenic disorder, such as a disorder characterized by the presence of tumor cells or immune cells expressing CD38 including, for example, hematological cancers such as B cell lymphoma, plasma cell malignancies, T/NK cell lymphoma, myeloid malignancies as well as solid tumor malignancies.

In some embodiments, the disease or disorder is a cancer involving tumor cells expressing CD38.

In some embodiments, the disease or disorder is a cancer involving immunosuppressive cells expressing CD38, such as non-cancerous immunosuppressive cells expressing CD38.

In some embodiments, the disease or disorder is a cancer involving both tumor cells and immunosuppressive cells expressing CD38.

In some embodiments, the disease or disorder is a cancer involving immunosuppressive cells expressing CD38 and tumor cells which do not express CD38.

In still other embodiments, the disease or disorder is an inflammatory and/or autoimmune disease or disorder involving cells expressing CD38.

In still other embodiments, the disease or disorder is a metabolic disorder involving cells expressing CD38.

### Hematological Cancers:

In one aspect, the disease or disorder is a hematological cancer. Examples of such hematological cancers include B cell lymphomas/leukemias including precursor B cell lymphoblastic leukemia/lymphoma and B cell non-Hodgkin's lymphomas; acute promyelocytic leukemia, acute lymphoblastic leukemia and mature B cell neoplasms, such as B cell chronic lymhocytic leukemia(CLL)/small lymphocytic lymphoma (SLL), B cell acute lymphocytic leukemia, B cell prolymphocytic leukemia, lymphoplasmacytic lymphoma, mantle cell lymphoma (MCL), follicular lymphoma (FL), including low-grade, intermediate-grade and high-grade FL, cutaneous follicle center lymphoma, marginal zone B cell lymphoma (MALT type, nodal and splenic type), hairy cell leukemia, diffuse large B cell lymphoma (DLBCL), Burkitt's lymphoma, plasmacytoma, plasma cell myeloma, plasma cell leukemia, post-transplant lymphoproliferative disorder, Waldenström's macroglobulinemia, plasma cell leukemias and anaplastic large-cell lymphoma (ALCL).

Examples of B cell non-Hodgkin's lymphomas are lymphomatoid granulomatosis, primary effusion lymphoma, intravascular large B cell lymphoma, mediastinal large B cell lymphoma, heavy chain diseases (including γ, μ, and α disease), lymphomas induced by therapy with immunosuppressive agents, such as cyclosporine-induced lymphoma, and methotrexate-induced lymphoma.

In one embodiment of the present invention, the disorder involving cells expressing CD38 is Hodgkin's lymphoma.

Other examples of disorders involving cells expressing CD38 include malignancies derived from T and NK cells including: mature T cell and NK cell neoplasms including T cell prolymphocytic leukemia, T cell large granular lymphocytic leukemia, aggressive NK cell leukemia, adult T cell leukemia/lymphoma, extranodal NK/T cell lymphoma, nasal type, enteropathy-type T cell lymphoma, hepatosplenic T cell lymphoma, subcutaneous panniculitis-like T cell lymphoma, blastic NK cell lymphoma, Mycosis Fungoides/¬Sézary Syndrome, primary cutaneous CD30 positive T cell lymphoproliferative disorders (primary cutaneous anaplastic large cell lymphoma C-ALCL, lymphomatoid papulosis, borderline lesions), angioimmunoblastic T cell lymphoma, peripheral T cell lymphoma unspecified, and anaplastic large cell lymphoma.

Examples of malignancies derived from myeloid cells include acute myeloid leukemia, including acute promyelocytic leukemia, and chronic myeloproliferative diseases, including chronic myeloid leukemia.

In some embodiments, the hematological cancer is selected from the group consisting of multiple myeloma (MM), chronic lymphocytic leukemia (CLL), acute lymphoblastic leukemia (ALL), acute myelogenous leukemia (adults) (AML), mantle cell lymphoma (MCL), follicular lymphoma (FL), and diffuse large B-cell lymphoma (DLBCL).

In some embodiments, the cancer is selected from the group consisting of multiple myeloma (MM), chronic lymphocytic leukemia (CLL), mantle cell lymphoma (MCL), diffuse large B-cell lymphoma (DLBCL), acute myelogenous leukemia (adults) (AML), acute lymphoblastic leukemia (ALL), and follicular lymphoma (FL).

In some embodiments, the cancer is multiple myeloma (MM).

In some embodiments, the cancer is chronic lymphocytic leukemia (CLL).

In some embodiments, the cancer is mantle cell lymphoma (MCL).

In some embodiments, the cancer is diffuse large B-cell lymphoma (DLBCL).

In some embodiments, the cancer is follicular lymphoma (FL).

In some embodiments, the cancer is acute myelogenous leukemia (adults) (AML).

In some embodiments, the cancer is acute lymphoblastic leukemia (ALL).

### Solid Tumor Malignancies:

In one aspect, the disease or disorder is a cancer comprising a solid tumor. That is, the patient suffering from cancer has a solid tumor.

Example of solid tumors include, but are not limited to, melanoma, lung cancer, squamous non-small cell lung cancer (NSCLC), non-squamous NSCLC, colorectal cancer, prostate cancer, castration-resistant prostate cancer, stomach cancer, ovarian cancer, gastric cancer, liver cancer, pancreatic cancer, thyroid cancer, squamous cell carcinoma of the head and neck, carcinoma of the esophagus or gastrointestinal tract, breast cancer, fallopian tube cancer, brain cancer, urethral cancer, genitourinary cancer, endometrial cancer, cervical cancer, lung adenocarcinoma, renal cell carcinoma (RCC) (e.g., a kidney clear cell carcinoma or a kidney papillary cell carcinoma), mesothelioma, nasopharyngeal carcinoma (NPC), a carcinomas of the esophagus or gastrointestinal tract, or a metastatic lesion of anyone thereof.

In one preferred embodiment, the solid tumor is from a cancer that contains immunosuppressive cells, such as Tregs, and that express CD38. T regulatory cells (Tregs) can have high expression of CD38, and Tregs with high CD38 expression are more immune suppressive compared to Tregs with intermediate CD38 expression (Krejcik J. et al. Blood 2016 128:384-394). Accordingly, without being limited to theory, the ability of antibody variants according to the invention to reduce the amount of CD38 expressed on Tregs via trogocytosis particularly allows for treatment of solid tumors in patients where the Tregs express CD38. Tregs express CD38 when CD38 expression on Tregs is statistically significant as compared to a control, e.g. expression detected with anti-CD38 antibody vs expression detected with an isotype control antibody using well known methods. This can be tested, e.g., by taking a biological sample such as a blood sample, bone marrow sample or a tumor biopsy.

So, in one aspect, the invention relates to the antibody variant according to any aspect or embodiment, or a pharmaceutical composition comprising the antibody variant, for use in the treatment or prevention of a solid tumor in a subject comprising Tregs expressing CD38.

In another aspect, the invention relates to a method of treating a solid tumor in a subject, comprising Tregs expressing CD38, the method comprising administering the antibody variant according to any aspect or embodiment to the subject, or a pharmaceutical composition comprising the antibody variant, typically in a therapeutically effective amount and/or for a time sufficient to treat the disease or disorder.

In some embodiments, the solid tumor is melanoma.

In some embodiments, the solid tumor is lung cancer.

In some embodiments, the solid tumor is squamous non-small cell lung cancer (NSCLC).

In some embodiments, the solid tumor is non-squamous NSCLC.

In some embodiments, the solid tumor is colorectal cancer.

In some embodiments, the solid tumor is prostate cancer.

In some embodiments, the solid tumor is castration-resistant prostate cancer.

In some embodiments, the solid tumor is stomach cancer.

In some embodiments, the solid tumor is ovarian cancer.

In some embodiments, the solid tumor is gastric cancer.

In some embodiments, the solid tumor is liver cancer.

In some embodiments, the solid tumor is pancreatic cancer.

In some embodiments, the solid tumor is thyroid cancer.

In some embodiments, the solid tumor is squamous cell carcinoma of the head and neck.

In some embodiments, the solid tumor is carcinoma of the esophagus or gastrointestinal tract.

In some embodiments, the solid tumor is breast cancer.

In some embodiments, the solid tumor is fallopian tube cancer.

In some embodiments, the solid tumor is brain cancer.

In some embodiments, the solid tumor is urethral cancer.

In some embodiments, the solid tumor is genitourinary cancer.

In some embodiments, the solid tumor is endometrial cancer.

In some embodiments, the solid tumor is cervical cancer.

In some embodiments, the tumor cells of the solid tumor lack detectable CD38 expression. The tumor cells of the solid tumor lack detectable CD38 expression when CD38 expression on tumor cells isolated from the solid tumor is statistically insignificant when compared to a control, e.g. expression detected with anti-CD38 antibody vs expression detected with an isotype control antibody using well known methods. This can be tested, e.g., by taking a biological sample such as a biopsy, from the tumor.

In some embodiments, the cancer is in a patient comprising T regulatory cells expressing CD38.

In specific embodiments, the antibody variant is administered in a therapeutically effective amount and/or for a sufficient period of time to treat the cancer.

### Metabolic Disorder:

In one aspect the disease or the disorder is a metabolic disorder. That is, the patient is suffering from a metabolic disorder.

In some embodiments the metabolic disorder is amyloidosis. Amyloidosis is a vast group of diseases defined by the presence of insoluble protein deposits in tissues. Its diagnosis is based on histological findings. In a further embodiment said amyloidosis may be AL amyloidosis.

### Patients:

The antibody variant of the present invention may be for the use of treatment or prevention of a disease or disorder in a subject who have received at least one prior therapy for the same disease or disorder with one or more compounds, wherein said one or more compounds are different from the antibody variant of the present invention. In one embodiment said disease or disorder may be any disease or disorder described herein; such as a cancer, inflammatory and/or autoimmune disease or disorder involving cells expressing CD38, or a metabolic disorder involving cells expressing CD38.

For example, in some embodiments the antibody variant of the present invention may be for the use of treatment or prevention of a disease or disorder in a subject who have received a prior treatment with a proteasome inhibitor (PI) and/or an immunomodulatory drug (IMiD). Examples of proteasome inhibitors include but are not limited to bortezomib, carfilzomib and ixazomib. Examples of IMiDs include but are not limited to thalidomide, lenalidomide and pomalidomide. In a further embodiment said disease or disorder may be a cancer or a tumor, such as multiple myeloma, mantle cell lymphoma or myelodysplastic syndrome (MDS). Thus the subject may be a cancer patient, such as a multiple myeloma, mantle cell lymphoma or myelodysplastic syndrome (MDS) patient.

The antibody variant of the present invention may be for the use of treatment or prevention of a disease or disorder in a subject which have not had any prior treatment with an anti-CD38 antibody. Typically, such a subject or patient is referred to as an anti-CD38 antibody naïve patient. In one embodiment the anti-CD38 antibody is daratumumab; i.e. the subject or patient have not had any prior treatment with daratumumab. Thus in one embodiment the subject or patient is a daratumumab-naïve subject/patient. The disease or disorder may be a cancer or tumor, or a metabolic disease, such amyloidosis, according to any aspect or embodiment disclosed herein.

The present invention also provides the antibody variant for the use of treatment or prevention of a disease or disorder in a subject who have received at least one prior therapy comprising a CD38 antibody.

The present invention also provides the antibody variant for use in treating cancer patients who have received at least one prior therapy comprising a CD38 antibody. The present invention also provides the antibody variant for use in treating patients with a metabolic disease, such as amyloidosis, who have received at least one prior therapy comprising a CD38 antibody. Such a prior therapy may have been one or more cycles of a planned treatment program comprising CD38 antibody, such as one or more planned cycles of CD38 antibody as single-agent therapy or in a combination therapy, as well as a sequence of treatments administered in a planned manner. In one embodiment, the prior therapy was CD38 antibody monotherapy. In one embodiment, the prior therapy was a combination therapy comprising a CD38 antibody. For example, the prior therapy may have been CD38 antibody in combination with a proteasome inhibitor (PI) and an immunomodulatory agent. In some embodiments, the CD38 antibody is daratumumab.

In some aspects, the cancer patient may also be one where administration of daratumumab as a monotherapy has a limited effect.

In some aspects, the cancer can be characterized as cancer that is “refractory” or “relapsed” to a prior therapy. In a further embodiment, the prior therapy may comprise one or more of a PI, an IMiD, and a CD38 antibody, e.g. wherein the CD38 antibody is daratumumab. Typically, this indicates that the prior therapy achieved less than a complete response (CR), for example, that the cancer was non-responsive to CD38 antibody mono- or combination therapy or that the cancer progressed within a predetermined period of time after the end of CD38 antibody therapy. Examples of such combination therapies include, but are not limited to, combination of a CD38 antibody with a PI or an IMiD or a combination of a PI and an IMiD. Similarly, it may indicate that that the prior therapy achieved less than a complete response (CR), for example, that the cancer was non-responsive to a PI, or an IMiD or a combination therapy thereof, or that the cancer progressed within a predetermined period of time after the end of said therapy. The skilled person can determine whether a cancer is refractory to a prior therapy based on what is known in the art, including guidelines available for each cancer.

For example, in multiple myeloma, refractory and relapsed disease can be identified according to the guidelines published by Rajkumar, Harousseau et al., on behalf of the International Myeloma Workshop Consensus Panel, Consensus recommendations for the uniform reporting of clinical trials: report of the International Myeloma Workshop Consensus Panel, Blood 2011; 117:4691-4695.

Refractory myeloma can be defined as disease that is nonresponsive while on primary or salvage therapy, or progresses within 60 days of last therapy. Nonresponsive disease is defined as either failure to achieve minimal response or development of progressive disease (PD) while on therapy. There may be 2 categories of refractory myeloma: “relapsed-and-refractory myeloma” and “primary refractory myeloma”.

Relapsed and refractory myeloma can be defined as disease that is nonresponsive while on salvage therapy, or progresses within 60 days of last therapy in patients who have achieved minimal response (MR) or better at some point previously before then progressing in their disease course.

Primary refractory myeloma can be defined as disease that is nonresponsive in patients who have never achieved a minimal response or better with any therapy. It includes patients who never achieve MR or better in whom there is no significant change in M protein and no evidence of clinical progression as well as primary refractory, PD where patients meet criteria for true PD. On reporting treatment efficacy for primary refractory patients, the efficacy in these 2 subgroups (“nonresponding-nonprogressive” and “progressive”) should be separately specified.

Relapsed myeloma can be defined as previously treated myeloma that progresses and requires the initiation of salvage therapy but does not meet criteria for either “primary refractory myeloma” or “relapsed-and-refractory myeloma” categories.

For details on specific responses (CR, PR etc.) in multiple myeloma and how to test them, see Rajkumar, Harousseau et al., 2011 (supra).

Accordingly, in some embodiments, the antibody variant according to any aspect or embodiment herein, or a pharmaceutical composition comprising the antibody variant, is for use in treating a cancer which is refractory to a prior treatment comprising one or more of a PI, an IMiD and a CD38 antibody. In one embodiment the prior treatment comprises a CD38 antibody. In a specific embodiment, the cancer is identified as a refractory cancer before the use.

In another embodiment, there is provided for a method for treating cancer in a subject, comprising the steps of:
(i) identifying the subject as being refractory to a prior treatment comprising one or more of a PI, an IMiD and a CD38 antibody, and
(ii) administering a therapeutically effective amount of the antibody variant according to any aspect or embodiment herein, or a pharmaceutical composition comprising the antibody variant to the subject.

In one embodiment the prior treatment comprises a CD38 antibody.

In another embodiment, there is provided for a method for treating cancer refractory to a prior treatment comprising one or more of a PI, an IMiD and a CD38 antibody in a subject, comprising administering a therapeutically effective amount of the antibody variant according to any aspect or embodiment herein, or a pharmaceutical composition comprising the antibody variant to the subject. In one embodiment the prior treatment comprises a CD38 antibody.

In some embodiments the PI is selected from the group consisting of bortezomib, carfilzomib and ixazomib.

In some embodiments the IMiD is selected from the group consisting of thalidomide, lenalidomide and pomalidomide.

In some embodiments, the CD38 antibody is daratumumab.

In some embodiments, the antibody variant according to any aspect or embodiment herein, or a pharmaceutical composition comprising the antibody variant, is for use in treating a cancer which is relapsed after a prior treatment comprising one or more of a PI, an IMiD and a CD38 antibody. In one embodiment the prior treatment comprises a CD38 antibody. In a specific embodiment, the cancer is identified as relapsed before the use.

In another embodiment, there is provided for a method for treating cancer in a subject, comprising the steps of:
(i) identifying the subject as being relapsed after a prior treatment comprising one or more of a PI, an IMiD and a CD38 antibody, and
(ii) administering a therapeutically effective amount of the antibody variant according to any aspect or embodiment herein, or a pharmaceutical composition comprising the antibody variant to the subject.

In one embodiment the prior treatment comprises a CD38 antibody.

In another embodiment, there is provided for a method for treating cancer relapsed after a prior treatment comprising one or more of a PI, an IMiD and a CD38 antibody in a subject, comprising administering a therapeutically effective amount of the antibody variant according to any aspect or embodiment herein, or a pharmaceutical composition comprising the antibody variant to the subject. In one embodiment the prior treatment comprises a CD38 antibody.

In some embodiments the PI is selected from the group consisting of bortezomib, carfilzomib and ixazomib.

In some embodiments the IMiD is selected from the group consisting of thalidomide, lenalidomide and pomalidomide.

In some embodiments, the CD38 antibody is daratumumab.

In specific embodiments, the antibody variant according to the present invention is administered in a therapeutically effective amount and/or for a sufficient period of time to treat the refractory or relapsed cancer.

In some embodiments, the refractory or relapsed cancer is a hematological cancer.

In some embodiments, the refractory or relapsed cancer is selected from the group consisting of multiple myeloma (MM), chronic lymphocytic leukemia (CLL), acute lymphoblastic leukemia (ALL), acute myelogenous leukemia (adults) (AML), mantle cell lymphoma (MCL), follicular lymphoma (FL), and diffuse large B-cell lymphoma (DLBCL).

In some embodiments, the refractory or relapsed cancer is selected from the group consisting of multiple myeloma (MM), chronic lymphocytic leukemia (CLL), mantle cell lymphoma (MCL), diffuse large B-cell lymphoma (DLBCL), and follicular lymphoma (FL).

In some embodiments, the refractory or relapsed cancer is multiple myeloma (MM).

In some embodiments, the refractory or relapsed cancer is chronic lymphocytic leukemia (CLL).

In some embodiments, the refractory or relapsed cancer is mantle cell lymphoma (MCL).

In some embodiments, the refractory or relapsed cancer is diffuse large B-cell lymphoma (DLBCL).

In some embodiments, the refractory or relapsed cancer is follicular lymphoma (FL).

In some embodiments, the refractory or relapsed cancer is a solid tumor. In some embodiments, the refractory or relapsed cancer is selected from the group consisting of melanoma, lung cancer, squamous non-small cell lung cancer (NSCLC), non-squamous NSCLC, colorectal cancer, prostate cancer, castration-resistant prostate cancer, stomach cancer, ovarian cancer, gastric cancer, liver cancer, pancreatic cancer, thyroid cancer, squamous cell carcinoma of the head and neck, carcinoma of the esophagus or gastrointestinal tract, breast cancer, fallopian tube cancer, brain cancer, urethral cancer, genitourinary cancer, endometrial cancer, cervical cancer.

In some embodiments, the refractory or relapsed cancer is melanoma.

In some embodiments, the refractory or relapsed cancer is lung cancer.

In some embodiments, the refractory or relapsed cancer is squamous non-small cell lung cancer (NSCLC).

In some embodiments, the refractory or relapsed cancer is non-squamous NSCLC.

In some embodiments, the refractory or relapsed cancer is colorectal cancer.

In some embodiments, the refractory or relapsed cancer is prostate cancer.

In some embodiments, the refractory or relapsed cancer is castration-resistant prostate cancer.

In some embodiments, the refractory or relapsed cancer is stomach cancer.

In some embodiments, the refractory or relapsed cancer is ovarian cancer.

In some embodiments, the refractory or relapsed cancer is gastric cancer.

In some embodiments, the refractory or relapsed cancer is liver cancer.

In some embodiments, the refractory or relapsed cancer is pancreatic cancer.

In some embodiments, the refractory or relapsed cancer is thyroid cancer.

In some embodiments, the refractory or relapsed cancer is squamous cell carcinoma of the head and neck.

In some embodiments, the refractory or relapsed cancer is carcinoma of the esophagus or gastrointestinal tract.

In some embodiments, the refractory or relapsed cancer is breast cancer.

In some embodiments, the refractory or relapsed cancer is fallopian tube cancer.

In some embodiments, the refractory or relapsed cancer is brain cancer.

In some embodiments, the refractory or relapsed cancer is urethral cancer.

In some embodiments, the refractory or relapsed cancer is genitourinary cancer.

In some embodiments, the refractory or relapsed cancer is endometrial cancer.

In some embodiments, the refractory or relapsed cancer is cervical cancer.

### Autoimmune and Inflammatory Diseases and Disorders:

In another embodiment of the present invention, the disorder involving cells expressing CD38 is an immune disorder in which CD38 expressing B cells, macrophages, plasma cells, monocytes and T cells are involved, such as an inflammatory and/or autoimmune disease. Examples of immune disorders in which CD38 expressing B cells, plasma cells, monocytes and T cells are involved include autoimmune disorders, such as psoriasis, psoriatic arthritis, dermatitis, systemic scleroderma and sclerosis, inflammatory bowel disease (IBD), Crohn's disease, ulcerative colitis, respiratory distress syndrome, meningitis, encephalitis, uveitis, glomerulonephritis, eczema, asthma, atherosclerosis, leukocyte adhesion deficiency, multiple sclerosis, Raynaud's syndrome, Sjögren's syndrome, juvenile onset diabetes, Reiter's disease, Behçet's disease, immune complex nephritis, IgA nephropathy, IgM polyneuropathies, immune-mediated thrombocytopenias, such as acute idiopathic thrombocytopenic purpura and chronic idiopathic thrombocytopenic purpura, hemolytic anemia, myasthenia gravis, lupus nephritis, systemic lupus erythematosus, rheumatoid arthritis (RA), atopic dermatitis, pemphigus, Graves' disease, Hashimoto's thyroiditis, Wegener's granulomatosis, Omenn's syndrome, chronic renal failure, acute infectious mononucleosis, multiple sclerosis, HIV, and herpes virus associated diseases. Further examples are severe acute respiratory distress syndrome and choreoretinitis. Furthermore, other diseases and disorders are included such as those caused by or mediated by infection of B-cells with virus, such as Epstein-Barr virus (EBV).

In one embodiment, the disorder involving cells expressing CD38 is rheumatoid arthritis.

Further examples of inflammatory, immune and/or autoimmune disorders in which autoantibodies and/or excessive B and T lymphocyte activity are prominent and which may be treated according to the present invention include the following: vasculitides and other vessel disorders, such as microscopic polyangiitis, Churg-Strauss syndrome, and other ANCA-associated vasculitides, polyarteritis nodosa, essential cryoglobulinaemic vasculitis, cutaneous leukocytoclastic angiitis, Kawasaki disease, Takayasu arteritis, giant cell arthritis, Henoch-Schönlein purpura, primary or isolated cerebral angiitis, erythema nodosum, thrombangiitis obliterans, thrombotic thrombocytopenic purpura (including hemolytic uremic syndrome), and secondary vasculitides, including cutaneous leukocytoclastic vasculitis (e.g., secondary to hepatitis B, hepatitis C, Waldenström's macroglobulinemia, B-cell neoplasias, rheumatoid arthritis, Sjögren's syndrome, or systemic lupus erythematosus); further examples are erythema nodosum, allergic vasculitis, panniculitis, Weber-Christian disease, purpura hyperglobulinaemica, and Buerger's disease; skin disorders, such as contact dermatitis, linear IgA dermatosis, vitiligo, pyoderma gangrenosum, epidermolysis bullosa acquisita, pemphigus vulgaris (including cicatricial pemphigoid and bullous pemphigoid), alopecia areata (including alopecia universalis and alopecia totalis), dermatitis herpetiformis, erythema multiforme, and chronic autoimmune urticaria (including angioneurotic edema and urticarial vasculitis); immune-mediated cytopenias, such as autoimmune neutropenia, and pure red cell aplasia; connective tissue disorders, such as CNS lupus, discoid lupus erythematosus, CREST syndrome, mixed connective tissue disease, polymyositis/dermatomyositis, inclusion body myositis, secondary amyloidosis, cryoglobulinemia type I and type II, fibromyalgia, phospholipid antibody syndrome, secondary hemophilia, relapsing polychondritis, sarcoidosis, stiff man syndrome, and rheumatic fever; a further example is eosinophil fasciitis; arthritides, such as ankylosing spondylitis, juvenile chronic arthritis, adult Still's disease, and SAPHO syndrome; further examples are sacroileitis, reactive arthritis, Still's disease, and gout; hematologic disorders, such as aplastic anemia, primary hemolytic anemia (including cold agglutinin syndrome), hemolytic anemia secondary to CLL or systemic lupus erythematosus; POEMS syndrome, pernicious anemia, and Waldenström's purpura hyperglobulinaemica; further examples are agranulocytosis, autoimmune neutropenia, Franklin's disease, Seligmann's disease, gamma heavy chain disease, paraneoplastic syndrome secondary to thymoma and lymphomas, an, paraneoplastic syndrome secondary to thymoma and lymphomas, and factor VIII inhibitor formation; endocrinopathies, such as polyendocrinopathy, and Addison's disease; further examples are autoimmune hypoglycemia, autoimmune hypothyroidism, autoimmune insulin syndrome, de Quervain's thyroiditis, and insulin receptor antibody-mediated insulin resistance; hepato-gastrointestinal disorders, such as celiac disease, Whipple's disease, primary biliary cirrhosis, chronic active hepatitis, and primary sclerosing cholangiitis; a further example is autoimmune gastritis; nephropathies, such as rapid progressive glomerulonephritis, post-streptococcal nephritis, Goodpasture's syndrome, membranous glomerulonephritis, and cryoglobulinemic nephritis; a further example is minimal change disease; neurological disorders, such as autoimmune neuropathies, mononeuritis multiplex, Lambert-Eaton's myasthenic syndrome, Sydenham's chorea, tabes dorsalis, and Guillain-Barré's syndrome; further examples are myelopathy/tropical spastic paraparesis, myasthenia gravis, acute inflammatory demyelinating polyneuropathy, and chronic inflammatory demyelinating polyneuropathy; multiple sclerosis; cardiac and pulmonary disorders, such as COPD, fibrosing alveolitis, bronchiolitis obliterans, allergic aspergillosis, cystic fibrosis, Löffler's syndrome, myocarditis, and pericarditis; further examples are hypersensitivity pneumonitis, and paraneoplastic syndrome secondary to lung cancer; allergic disorders, such as bronchial asthma and hyper-IgE syndrome; a further example is amaurosis fugax; ophthalmologic disorders, such as idiopathic chorioretinitis; infectious diseases, such as parvovirus B infection (including hands-and-socks syndrome); gynecological-obstretical disorders, such as recurrent abortion, recurrent fetal loss, and intrauterine growth retardation; a further example is paraneoplastic syndrome secondary to gynaecological neoplasms; male reproductive disorders, such as paraneoplastic syndrome secondary to testicular neoplasms; and transplantation-derived disorders, such as allograft and xenograft rejection, and graft-versus-host disease.

In one embodiment, the disease or disorder is rheumatoid arthritis.

### Dosage Regimens and Combinations

Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions may be formulated in dosage unit form for ease of administration and uniformity of dosage.

The efficient dosages and the dosage regimens for the antibody variants depend on the disease or condition to be treated and may be determined by the persons skilled in the art. An exemplary, non-limiting range for a therapeutically effective amount of an antibody variant of the present invention is about 0.001-30 mg/kg.

An antibody variant may also be administered prophylactically in order to reduce the risk of developing cancer, delay the onset of the occurrence of an event in cancer progression, and/or reduce the risk of recurrence when a cancer is in remission.

An antibody variant may also be administered in a combination therapy, i.e., combined with other therapeutic agents or therapeutic modalities relevant for the disease or condition to be treated.

Accordingly, in one embodiment, the antibody variant is for combination with one or more further therapeutic agents, such as a chemotherapeutic agent, an anti-inflammatory agent, or an immunosuppressive and/or immunomodulatory agent, e.g., another therapeutic antibody. Such combined administration may be simultaneous, separate or sequential. For simultaneous administration the agents may be administered as one composition or as separate compositions, as appropriate.

The antibody variant may also be used in combination with radiotherapy and/or surgery and/or autologous or allogeneic peripheral stem cell or bone marrow transplantation.

### Diagnostic Applications

In further aspects, diagnostic compositions and uses comprising the antibody variant according to any aspect or embodiment are also contemplated, e.g., for diseases involving cells expressing CD38, as exemplified above. The antibody variant may, for example, be labelled with a radioactive agent (as described elsewhere herein) or a radioopaque agent. In one embodiment, the diagnostic composition is a companion diagnostic which is used to screen and select those patients who will benefit from treatment with the antibody variant.

In one embodiment, the present invention relates to use of an antibody variant, composition or kit-of-parts according to any aspect or embodiment herein for use in a diagnostic method.

In one embodiment, the present invention relates to a diagnostic method comprising administering a polypeptide, antibody, a composition or a kit-of-parts according to any aspect or embodiment herein to at least a part of the body of a human or other mammal.

In another embodiment, the present invention relates to use of an antibody variant, composition or kit-of-parts according to any of the aspects or embodiments herein in imaging of at least a part of the body of a human or other mammal.

In another embodiment, the present invention relates to a method for imaging of at least a part of the body of a human or other mammal, comprising administering a variant, a composition or a kit-of-parts according to any aspect or embodiments herein described.

TABLE 1
Amino acid and nucleic acid sequences

| SEQ ID | | |
|---|---|---|
| NO: | DESIGNATION | SEQUENCE |
| 1 | VH-3003-C | QVQLVQSGAEVKKPGSSVKVSCKAFGGTFSSYAISWVRQAPGQ |
| | | GLEWMGRIIRFLGIANYAQKFQGRVTLIADKSTNTAYMELSSL |
| | | RSEDTAVYYCAGEPGERDPDAVDIWGQGTMVTVSS |
| 2 | VH-3003-C_CDR1 | GGTFSSYA |
| 3 | VH-3003-C_CDR2 | IIRFLGIA |
| 4 | VH-3003-C_CDR3 | AGEPGERDPDAVDI |
| 5 | VL(Kappa)-3003-C | DIQMTQSPSSLSASVGDRVTITCRASQGIRSWLAWYQQKPEKA |
| | | PKSLIYAASSLQSGVPSRFSGSGSGTDFTLTISSLQPEDFATY |
| | | YCQQYNSYPLTFGGGTKVEIK |
| 6 | VL(Kappa)-3003- | QGIRSW |
| | C_CDR1 | |
| | VL(Kappa)-3003- | AAS |
| | C_CDR2 | |
| 7 | VL(Kappa)-3003- | QQYNSYPLT |
| | C_CDR3 | |
| 8 | VH-3003-B | EVQLLESGGGLVQPGGSLRLSCAVSGFTFNSFAMSWVRQAPGK |
| | | GLEWVSAISGSGGGTYYADSVKGRFTISRDNSKNTLYLQMNSL |
| | | RAEDTAVYFCAKDKILWFGEPVFDYWGQGTLVTVSS |
| 9 | VL(Kappa)-3003-B | EIVLTQSPATLSLSPGERATLSCRASQSVSSYLAWYQQKPGQA |
| | | PRLLIYDASNRATGIPARFSGSGSGTDFTLTISSLEPEDFAVY |
| | | YCQQRSNWPPTFGQGTKVEIK |
| 10 | VH-3003-A | QVQLVQSGAEVKKPGSSVKVSCKASGGTFSSYAFSWVRQAPGQ |
| | | GLEWMGRVIPFLGIANSAQKFQGRVTITADKSTSTAYMDLSSL |
| | | RSEDTAVYYCARDDIAALGPFDYWGQGTLVTVSS |
| 11 | VL(Kappa)-3003-A | DIQMTQSPSSLSASVGDRVTITCRASQGISSWLAWYQQKPEKA |
| | | PKSLIYAASSLQSGVPSRFSGSGSGTDFTLTISSLQPEDFATY |
| | | YCQQYNSYPRTFGQGTKVEIK |
| 12 | VH-gp120-b12 | QVQLVQSGAEVKKPGASVKVSCQASGYRFSNFVIHWVRQAPGQ |
| | | RFEWMGWINPYNGNKEFSAKFQDRVTFTADTSANTAYMELRSL |
| | | RSADTAVYYCARVGPYSWDDSPQDNYYMDVWGKGTTVIVSS |
| 13 | VH-gp120-b12_CDR1 | GYRFSNFV |
| 14 | VH-gp120-b12_CDR2 | INPYNGNK |
| 15 | VH-gp120-b12_CDR3 | ARVGPYSWDDSPQDNYYMDV |
| 16 | VL-gp120-b12 | EIVLTQSPGTLSLSPGERATFSCRSSHSIRSRRVAWYQHKPGQ |
| | | APRLVIHGVSNRASGISDRFSGSGSGTDFTLTITRVEPEDFAL |
| | | YYCQVYGASSYTFGQGTKLERK |
| 17 | VL-gp120-b12_CDR1 | HSIRSRR |
| | VL-gp120-b12_CDR2 | GVS |
| 18 | VL-gp120-b12_CDR3 | QVYGASSYT |
| 19 | constant region | ASTKGPSVFPLAPSSKSTSGGTAALGCLVKDYFPEPVTVSWNS |
| | human HC IgG1m(za) | GALTSGVHTFPAVLQSSGLYSLSSVVTVPSSSLGTQTYICNVN |
| | (Uniprot entry | HKPSNTKVDKKVEPKSCDKTHTCPPCPAPELLGGPSVFLFPPK |
| | P01857) | PKDTLMISRTPEVTCVVVDVSHEDPEVKFNWYVDGVEVHNAKT |
| | | KPREEQYNSTYRVVSVLTVLHQDWLNGKEYKCKVSNKALPAPI |
| | | EKTISKAKGQPREPQVYTLPPSRDELTKNQVSLTCLVKGFYPS |
| | | DIAVEWESNGQPENNYKTTPPVLDSDGSFFLYSKLTVDKSRWQ |
| | | QGNVFSCSVMHEALHNHYTQKSLSLSPGK |
| 20 | constant region | ASTKGPSVFPLAPSSKSTSGGTAALGCLVKDYFPEPVTVSWNS |
| | human HC IgG1m(f) | GALTSGVHTFPAVLQSSGLYSLSSVVTVPSSSLGTQTYICNVN |
| | | HKPSNTKVDKRVEPKSCDKTHTCPPCPAPELLGGPSVFLFPPK |
| | | PKDTLMISRTPEVTCVVVDVSHEDPEVKFNWYVDGVEVHNAKT |
| | | KPREEQYNSTYRVVSVLTVLHQDWLNGKEYKCKVSNKALPAPI |
| | | EKTISKAKGQPREPQVYTLPPSREEMTKNQVSLTCLVKGFYPS |
| | | DIAVEWESNGQPENNYKTTPPVLDSDGSFFLYSKLTVDKSRWQ |
| | | QGNVFSCSVMHEALHNHYTQKSLSLSPGK |
| 21 | constant region | ASTKGPSVFPLAPSSKSTSGGTAALGCLVKDYFPEPVTVSWNS |
| | human HC IgG1m(z) | GALTSGVHTFPAVLQSSGLYSLSSVVTVPSSSLGTQTYICNVN |
| | | HKPSNTKVDKKVEPKSCDKTHTCPPCPAPELLGGPSVFLFPPK |
| | | PKDTLMISRTPEVTCVVVDVSHEDPEVKFNWYVDGVEVHNAKT |
| | | KPREEQYNSTYRVVSVLTVLHQDWLNGKEYKCKVSNKALPAPI |
| | | EKTISKAKGQPREPQVYTLPPSREEMTKNQVSLTCLVKGFYPS |
| | | DIAVEWESNGQPENNYKTTPPVLDSDGSFFLYSKLTVDKSRWQ |
| | | QGNVFSCSVMHEALHNHYTQKSLSLSPGK |
| 22 | constant region | ASTKGPSVFPLAPSSKSTSGGTAALGCLVKDYFPEPVTVSWNS |
| | human HC IgG1m(a) | GALTSGVHTFPAVLQSSGLYSLSSVVTVPSSSLGTQTYICNVN |
| | | HKPSNTKVDKPVEPKSCDKTHTCPPCPAPELLGGPSVFLFPPK |
| | | PKDTLMISRTPEVTCVVVDVSHEDPEVKFNWYVDGVEVHNAKT |
| | | KPREEQYNSTYRVVSVLTVLHQDWLNGKEYKCKVSNKALPAPI |
| | | EKTISKAKGQPREPQVYTLPPSRDELTKNQVSLTCLVKGFYPS |
| | | DIAVEWESNGQPENNYKTTPPVLDSDGSFFLYSKLTVDKSRWQ |
| | | QGNVFSCSVMHEALHNHYTQKSLSLSPGK |
| 23 | constant region | ASTKGPSVFPLAPSSKSTSGGTAALGCLVKDYFPEPVTVSWNS |
| | human HC IgG1m(x) | GALTSGVHTFPAVLQSSGLYSLSSVVTVPSSSLGTQTYICNVN |
| | | HKPSNTKVDKPVEPKSCDKTHTCPPCPAPELLGGPSVFLFPPK |
| | | PKDTLMISRTPEVTCVVVDVSHEDPEVKFNWYVDGVEVHNAKT |
| | | KPREEQYNSTYRVVSVLTVLHQDWLNGKEYKCKVSNKALPAPI |
| | | EKTISKAKGQPREPQVYTLPPSREEMTKNQVSLTCLVKGFYPS |
| | | DIAVEWESNGQPENNYKTTPPVLDSDGSFFLYSKLTVDKSRWQ |
| | | QGNVFSCSVMHEGLHNHYTQKSLSLSPGK |
| 24 | constant region | ASTKGPSVFPLAPSSKSTSGGTAALGCLVKDYFPEPVTVSWNS |
| | human HC IgG1m(f)- | GALTSGVHTFPAVLQSSGLYSLSSVVTVPSSSLGTQTYICNVN |
| | E430G | HKPSNTKVDKRVEPKSCDKTHTCPPCPAPELLGGPSVFLFPPK |
| | | PKDTLMISRTPEVTCVVVDVSHEDPEVKFNWYVDGVEVHNAKT |
| | | KPREEQYNSTYRVVSVLTVLHQDWLNGKEYKCKVSNKALPAPI |
| | | EKTISKAKGQPREPQVYTLPPSREEMTKNQVSLTCLVKGFYPS |
| | | DIAVEWESNGQPENNYKTTPPVLDSDGSFFLYSKLTVDKSRWQ |
| | | QGNVFSCSVMHGALHNHYTQKSLSLSPGK |
| 25 | constant region | ASTKGPSVFPLAPSSKSTSGGTAALGCLVKDYFPEPVTVSWNS |
| | human HC IgG1m(f)- | GALTSGVHTFPAVLQSSGLYSLSSVVTVPSSSLGTQTYICNVN |
| | E430S | HKPSNTKVDKRVEPKSCDKTHTCPPCPAPELLGGPSVFLFPPK |
| | | PKDTLMISRTPEVTCVVVDVSHEDPEVKFNWYVDGVEVHNAKT |
| | | KPREEQYNSTYRVVSVLTVLHQDWLNGKEYKCKVSNKALPAPI |
| | | EKTISKAKGQPREPQVYTLPPSREEMTKNQVSLTCLVKGFYPS |
| | | DIAVEWESNGQPENNYKTTPPVLDSDGSFFLYSKLTVDKSRWQ |
| | | QGNVFSCSVMHSALHNHYTQKSLSLSPGK |
| 26 | constant region | ASTKGPSVFPLAPSSKSTSGGTAALGCLVKDYFPEPVTVSWNS |
| | human HC IgG1m(f)- | GALTSGVHTFPAVLQSSGLYSLSSVVTVPSSSLGTQTYICNVN |
| | E430F | HKPSNTKVDKRVEPKSCDKTHTCPPCPAPELLGGPSVFLFPPK |
| | | PKDTLMISRTPEVTCVVVDVSHEDPEVKFNWYVDGVEVHNAKT |
| | | KPREEQYNSTYRVVSVLTVLHQDWLNGKEYKCKVSNKALPAPI |
| | | EKTISKAKGQPREPQVYTLPPSREEMTKNQVSLTCLVKGFYPS |
| | | DIAVEWESNGQPENNYKTTPPVLDSDGSFFLYSKLTVDKSRWQ |
| | | QGNVFSCSVMHFALHNHYTQKSLSLSPGK |
| 27 | constant region | ASTKGPSVFPLAPSSKSTSGGTAALGCLVKDYFPEPVTVSWNS |
| | human HC IgG1m(f)- | GALTSGVHTFPAVLQSSGLYSLSSVVTVPSSSLGTQTYICNVN |
| | E430T | HKPSNTKVDKRVEPKSCDKTHTCPPCPAPELLGGPSVFLFPPK |
| | | PKDTLMISRTPEVTCVVVDVSHEDPEVKFNWYVDGVEVHNAKT |
| | | KPREEQYNSTYRVVSVLTVLHQDWLNGKEYKCKVSNKALPAPI |
| | | EKTISKAKGQPREPQVYTLPPSREEMTKNQVSLTCLVKGFYPS |
| | | DIAVEWESNGQPENNYKTTPPVLDSDGSFFLYSKLTVDKSRWQ |
| | | QGNVFSCSVMHTALHNHYTQKSLSLSPGK |
| 28 | constant region | ASTKGPSVFPLAPSSKSTSGGTAALGCLVKDYFPEPVTVSWNS |
| | human HC IgG1m(f)- | GALTSGVHTFPAVLQSSGLYSLSSVVTVPSSSLGTQTYICNVN |
| | E345K | HKPSNTKVDKRVEPKSCDKTHTCPPCPAPELLGGPSVFLFPPK |
| | | PKDTLMISRTPEVTCVVVDVSHEDPEVKFNWYVDGVEVHNAKT |
| | | KPREEQYNSTYRVVSVLTVLHQDWLNGKEYKCKVSNKALPAPI |
| | | EKTISKAKGQPRKPQVYTLPPSREEMTKNQVSLTCLVKGFYPS |
| | | DIAVEWESNGQPENNYKTTPPVLDSDGSFFLYSKLTVDKSRWQ |
| | | QGNVFSCSVMHEALHNHYTQKSLSLSPGK |
| 29 | constant region | ASTKGPSVFPLAPSSKSTSGGTAALGCLVKDYFPEPVTVSWNS |
| | human HC IgG1m(f)- | GALTSGVHTFPAVLQSSGLYSLSSVVTVPSSSLGTQTYICNVN |
| | E345Q | HKPSNTKVDKRVEPKSCDKTHTCPPCPAPELLGGPSVFLFPPK |
| | | PKDTLMISRTPEVTCVVVDVSHEDPEVKFNWYVDGVEVHNAKT |
| | | KPREEQYNSTYRVVSVLTVLHQDWLNGKEYKCKVSNKALPAPI |
| | | EKTISKAKGQPRQPQVYTLPPSREEMTKNQVSLTCLVKGFYPS |
| | | DIAVEWESNGQPENNYKTTPPVLDSDGSFFLYSKLTVDKSRWQ |
| | | QGNVFSCSVMHEALHNHYTQKSLSLSPGK |
| 30 | constant region | ASTKGPSVFPLAPSSKSTSGGTAALGCLVKDYFPEPVTVSWNS |
| | human HC IgG1m(f)- | GALTSGVHTFPAVLQSSGLYSLSSVVTVPSSSLGTQTYICNVN |
| | E345R | HKPSNTKVDKRVEPKSCDKTHTCPPCPAPELLGGPSVFLFPPK |
| | | PKDTLMISRTPEVTCVVVDVSHEDPEVKFNWYVDGVEVHNAKT |
| | | KPREEQYNSTYRVVSVLTVLHQDWLNGKEYKCKVSNKALPAPI |
| | | EKTISKAKGQPRRPQVYTLPPSREEMTKNQVSLTCLVKGFYPS |
| | | DIAVEWESNGQPENNYKTTPPVLDSDGSFFLYSKLTVDKSRWQ |
| | | QGNVFSCSVMHEALHNHYTQKSLSLSPGK |
| 31 | constant region | ASTKGPSVFPLAPSSKSTSGGTAALGCLVKDYFPEPVTVSWNS |
| | human HC IgG1m(f)- | GALTSGVHTFPAVLQSSGLYSLSSVVTVPSSSLGTQTYICNVN |
| | E345Y | HKPSNTKVDKRVEPKSCDKTHTCPPCPAPELLGGPSVFLFPPK |
| | | PKDTLMISRTPEVTCVVVDVSHEDPEVKFNWYVDGVEVHNAKT |
| | | KPREEQYNSTYRVVSVLTVLHQDWLNGKEYKCKVSNKALPAPI |
| | | EKTISKAKGQPRYPQVYTLPPSREEMTKNQVSLTCLVKGFYPS |
| | | DIAVEWESNGQPENNYKTTPPVLDSDGSFFLYSKLTVDKSRWQ |
| | | QGNVFSCSVMHEALHNHYTQKSLSLSPGK |
| 32 | constant region | ASTKGPSVFPLAPSSKSTSGGTAALGCLVKDYFPEPVTVSWNS |
| | human HC IgG1m(f)- | GALTSGVHTFPAVLQSSGLYSLSSVVTVPSSSLGTQTYICNVN |
| | S440W | HKPSNTKVDKRVEPKSCDKTHTCPPCPAPELLGGPSVFLFPPK |
| | | PKDTLMISRTPEVTCVVVDVSHEDPEVKFNWYVDGVEVHNAKT |
| | | KPREEQYNSTYRVVSVLTVLHQDWLNGKEYKCKVSNKALPAPI |
| | | EKTISKAKGQPREPQVYTLPPSREEMTKNQVSLTCLVKGFYPS |
| | | DIAVEWESNGQPENNYKTTPPVLDSDGSFFLYSKLTVDKSRWQ |
| | | QGNVFSCSVMHEALHNHYTQKWLSLSPGK |
| 33 | constant region | ASTKGPSVFPLAPSSKSTSGGTAALGCLVKDYFPEPVTVSWNS |
| | human HC IgG1m(f)- | GALTSGVHTFPAVLQSSGLYSLSSVVTVPSSSLGTQTYICNVN |
| | S440Y | HKPSNTKVDKRVEPKSCDKTHTCPPCPAPELLGGPSVFLFPPK |
| | | PKDTLMISRTPEVTCVVVDVSHEDPEVKFNWYVDGVEVHNAKT |
| | | KPREEQYNSTYRVVSVLTVLHQDWLNGKEYKCKVSNKALPAPI |
| | | EKTISKAKGQPREPQVYTLPPSREEMTKNQVSLTCLVKGFYPS |
| | | DIAVEWESNGQPENNYKTTPPVLDSDGSFFLYSKLTVDKSRWQ |
| | | QGNVFSCSVMHEALHNHYTQKYLSLSPGK |
| 34 | constant region | ASTKGPSVFPLAPCSRSTSESTAALGCLVKDYFPEPVTVSWNS |
| | human HC IgG2 | GALTSGVHTFPAVLQSSGLYSLSSVVTVPSSNFGTQTYTCNVD |
| | (Uniprot entry | HKPSNTKVDKTVERKCCVECPPCPAPPVAGPSVFLFPPKPKDT |
| | P01859) | LMISRTPEVTCVVVDVSHEDPEVQFNWYVDGVEVHNAKTKPRE |
| | | EQFNSTFRVVSVLTVVHQDWLNGKEYKCKVSNKGLPAPIEKTI |
| | | SKTKGQPREPQVYTLPPSREEMTKNQVSLTCLVKGFYPSDIAV |
| | | EWESNGQPENNYKTTPPMLDSDGSFFLYSKLTVDKSRWQQGNV |
| | | FSCSVMHEALHNHYTQKSLSLSPGK |
| 35 | constant region | ASTKGPSVFPLAPCSRSTSGGTAALGCLVKDYFPEPVTVSWNS |
| | human HC IgG3 | GALTSGVHTFPAVLQSSGLYSLSSVVTVPSSSLGTQTYTCNVN |
| | (Uniprot entry | HKPSNTKVDKRVELKTPLGDTTHTCPRCPEPKSCDTPPPCPRC |
| | P01860) | PEPKSCDTPPPCPRCPEPKSCDTPPPCPRCPAPELLGGPSVFL |
| | | FPPKPKDTLMISRTPEVTCVVVDVSHEDPEVQFKWYVDGVEVH |
| | | NAKTKPREEQYNSTFRVVSVLTVLHQDWLNGKEYKCKVSNKAL |
| | | PAPIEKTISKTKGQPREPQVYTLPPSREEMTKNQVSLTCLVKG |
| | | FYPSDIAVEWESSGQPENNYNTTPPMLDSDGSFFLYSKLTVDK |
| | | SRWQQGNIFSCSVMHEALHNRFTQKSLSLSPGK |
| 36 | constant region | ASTKGPSVFPLAPCSRSTSESTAALGCLVKDYFPEPVTVSWNS |
| | human HC IgG4 | GALTSGVHTFPAVLQSSGLYSLSSVVTVPSSSLGTKTYTCNVD |
| | (Uniprot entry | HKPSNTKVDKRVESKYGPPCPSCPAPEFLGGPSVFLFPPKPKD |
| | P01861) | TLMISRTPEVTCVVVDVSQEDPEVQFNWYVDGVEVHNAKTKPR |
| | | EEQFNSTYRVVSVLTVLHQDWLNGKEYKCKVSNKGLPSSIEKT |
| | | ISKAKGQPREPQVYTLPPSQEEMTKNQVSLTCLVKGFYPSDIA |
| | | VEWESNGQPENNYKTTPPVLDSDGSFFLYSRLTVDKSRWQEGN |
| | | VFSCSVMHEALHNHYTQKSLSLSLGK |
| 37 | constant region | RTVAAPSVFIFPPSDEQLKSGTASVVCLLNNFYPREAKVQWKV |
| | human Kappa LC | DNALQSGNSQESVTEQDSKDSTYSLSSTLTLSKADYEKHKVYA |
| | | CEVTHQGLSSPVTKSFNRGEC |
| 38 | Human CD38 (Uniprot | MANCEFSPVSGDKPCCRLSRRAQLCLGVSILVLILVVVLAVVV |
| | entry P28907) | PRWRQQWSGPGTTKRFPETVLARCVKYTEIHPEMRHVDCQSVW |
| | | DAFKGAFISKHPCNITEEDYQPLMKLGTQTVPCNKILLWSRIK |
| | | DLAHQFTQVQRDMFTLEDTLLGYLADDLTWCGEFNTSKINYQS |
| | | CPDWRKDCSNNPVSVFWKTVSRRFAEAACDVVHVMLNGSRSKI |
| | | FDKNSTFGSVEVHNLQPEKVQTLEAWVIHGGREDSRDLCQDPT |
| | | IKELESIISKRNIQFSCKNIYRPDKFLQCVKNPEDSSCTSEI |
| 39 | hisCD38 | HHHHHHRWRQTWSGPGTTKRFPETVLARCVKYTEIHPEMRHVD |
| | | CQSVWDAFKGAFISKHPCNITEEDYQPLMKLGTQTVPCNKILL |
| | | WSRIKDLAHQFTQVQRDMFTLEDTLLGYLADDLTWCGEFNTSK |
| | | INYQSCPDWRKDCSNNPVSVFWKTVSRRFAEAACDVVHVMLNG |
| | | SRSKIFDKNSTFGSVEVHNLQPEKVQTLEAWVIHGGREDSRDL |
| | | CQDPTIKELESIISKRNIQFSCKNIYRPDKFLQCVKNPEDSSC |
| | | TSEI |
| 40 | VH CDR1 variants | GGTFX₁SYA, wherein X₁ is S or R |
| 41 | VH CDR2 variants | IIX₁FLGX₂X₃, wherein X₁ is R or V; X₂ is I or |
| | | K; and X₃ is A, T or V, such as A or T |
| 42 | VH CDR3 variants | X₁GEPGX₂RDPDAX₃DI, wherein X₁ is A or T; X₂ |
| | | is E, D or A, such as E or D; and X₃ is V |
| | | or F |
| 43 | VL CDR1 | QGIRSW |
| | VL CDR2 | AAS |
| 44 | VL CDR3 variants | QQYNX₁YPLT, wherein X₁ is S or N |
| 45 | constant region | ASTKGPSVFPLAPSSKSTSGGTAALGCLVKDYFPEPVTVSWNS |
| | human HC IgG1m(f) | GALTSGVHTFPAVLQSSGLYSLSSVVTVPSSSLGTQTYICNVN |
| | without Lys (K) at | HKPSNTKVDKRVEPKSCDKTHTCPPCPAPELLGGPSVFLFPPK |
| | position 447 | PKDTLMISRTPEVTCVVVDVSHEDPEVKFNWYVDGVEVHNAKT |
| | according to Eu | KPREEQYNSTYRVVSVLTVLHQDWLNGKEYKCKVSNKALPAPI |
| | numbering | EKTISKAKGQPREPQVYTLPPSREEMTKNQVSLTCLVKGFYPS |
| | | DIAVEWESNGQPENNYKTTPPVLDSDGSFFLYSKLTVDKSRWQ |
| | | QGNVFSCSVMHEALHNHYTQKSLSLSPG |
| 46 | constant region | ASTKGPSVFPLAPSSKSTSGGTAALGCLVKDYFPEPVTVSWNS |
| | human HC IgG1m(f)- | GALTSGVHTFPAVLQSSGLYSLSSVVTVPSSSLGTQTYICNVN |
| | E430G, without Lys | HKPSNTKVDKRVEPKSCDKTHTCPPCPAPELLGGPSVFLFPPK |
| | (K) at position 447 | PKDTLMISRTPEVTCVVVDVSHEDPEVKFNWYVDGVEVHNAKT |
| | according to Eu | KPREEQYNSTYRVVSVLTVLHQDWLNGKEYKCKVSNKALPAPI |
| | numbering | EKTISKAKGQPREPQVYTLPPSREEMTKNQVSLTCLVKGFYPS |
| | | DIAVEWESNGQPENNYKTTPPVLDSDGSFFLYSKLTVDKSRWQ |
| | | QGNVFSCSVMHGALHNHYTQKSLSLSPG |

### EXAMPLES

The present invention is further illustrated by the following examples which should not be construed as limiting.

### Example 1—Antibodies and Cell-Lines

### Antibody Expression Constructs

For the expression of human and humanized antibodies used herein, variable heavy (VH) chain and variable light (VL) chain sequences were prepared by gene synthesis (GeneArt Gene Synthesis; ThermoFisher Scientific) and cloned in pcDNA3.3 expression vectors (ThermoFisher Scientific) containing a constant region of a human IgG heavy chain (HC) (constant region human IgG1m(f) HC: SEQ ID NO:20) and/or the constant region of the human kappa light chain (LC): SEQ ID NO:37. Desired mutations were introduced by gene synthesis. CD38 antibody variants in this application have VH and VL sequences derived from previously described CD38 antibodies IgG1-A (WO 2006/099875 A1, WO 2008/037257 A2, WO 2011/154453 A1; VH: SEQ ID NO:10; VL: SEQ ID NO:11), IgG1-B (WO 2006/099875 A1, WO 2008/037257 A2, WO 2011/154453 A1; VH: SEQ ID NO:8; VL: SEQ ID NO:9), and IgG1-C (WO 2011/154453 A1; VH: SEQ ID NO:1; VL: SEQ ID NO:5). The human IgG1 antibody b12, an HIV gp120-specific antibody was used as a negative control in some experiments (Barbas et al., J Mol Biol. 1993 Apr. 5; 230(3):812-23; VH: SEQ ID NO:12; VL: SEQ ID NO:16).

### Transient Expression Antibody Constructs

Plasmid DNA mixtures encoding both heavy and light chains of antibodies were transiently transfected in Expi293F cells (Gibco, Cat No A14635) using 293fectin (Life Technologies) essentially as described by Vink et al. (Vink et al., 2014 Methods 65(1):5-10). Antibody concentrations in the supernatants were measured by absorbance at 280 nm. Antibody-containing supernatants were either directly used in in vitro assays, or antibodies were purified as described below.

### Antibody Purification and Quality Assessment

Antibodies were purified by Protein A affinity chromatography. Culture supernatants were filtered over a 0.20 μM dead-end filter and loaded on 5 mL MabSelect SuRe columns (GE Healthcare), washed and eluted with 0.02 M sodium citrate-NaOH, pH 3. The eluates were loaded on a HiPrep Desalting column (GE Healthcare) immediately after purification and the antibodies were buffer exchanged into 12.6 mM NaH2PO4, 140 mM NaCl, pH 7.4 buffer (B. Braun or Thermo Fisher). After buffer exchange, samples were sterile filtered over 0.2 μm dead-end filters. Purified proteins were analyzed by a number of bioanalytical assays including capillary electrophoresis on sodium dodecyl sulfate-polyacrylamide gels (CE-SDS) and high-performance size exclusion chromatography (HP-SEC). Concentration was measured by absorbance at 280 nm. Purified antibodies were stored at 2-8° C.

The cell-lines used in the Examples are described in Table 2 below. The average number of CD38 and CD59-molecules per cell was determined by quantitative flow cytometry (Qifi, DAKO).

TABLE 2
Overview of cell lines and expression of CD38 and CD59

| | tumor | | | Estimated ABCs | |
|---|---|---|---|---|---|
| cell line | type | Catalog | supplier | CD38 | CD59 |
| SU-DHL-8 | DLBCL | ACC 573 | DSMZ | 415000 | 31000 |
| Oci-Ly-7 | DLBCL | ACC 688 | DSMZ | 310000 | 81000 |
| Oci-Ly-19 | DLBCL | ACC 528 | DSMZ | 271000 | 28000 |
| Ramos | Burkitt | CRL-1596 | ATCC | 260000 | 7000 |
| Daudi | Burkitt | CCL-213 | ATCC | 200000 | 0 |
| Oci-Ly18 | DLBCL | ACC 699 | DSMZ | 181000 | 40000 |
| Raji | Burkitt | CCL-86 | ATCC | 170000 | 55000 |
| DOHH2 | FL | ACC 47 | DSMZ | 167000 | 66000 |
| SU-DHL-4 | DLBCL | ACC 495 | DSMZ | 158000 | 147000 |
| WSU-DLCL2 | DLBCL | ACC 575 | DSMZ | 150000 | 96000 |
| Z-138 | MCL | CRL-3001 | ATCC | 133000 | 53000 |
| JVM-13 | MCL | CRL-3003 | ATCC | 130000 | 254000 |
| REH | B-ALL | ACC 22 | DSMZ | 130000 | not tested |
| Jeko-1 | MCL | ACC 553 | DSMZ | 108000 | 31000 |
| Wien133 | Burkitt | — | BioAnaLab, | 100000 | 0 |
| | | | UK | | |
| 697 | B-ALL | ACC 42 | DSMZ | 98000 | 130000 |
| Granta-519 | MCL | ACC 342 | DSMZ | 90000 | 140000 |
| RS4; 11 | B-ALL | ACC 508 | DSMZ | 80-86000 | not tested |
| DB | DLBCL | ACC 539 | DSMZ | 70000 | 200000 |
| NALM-16 | B-ALL | ACC 680 | DSMZ | 50000 | not tested |
| JVM-3 | CLL | ACC 18 | DSMZ | 30000 | not tested |
| U266 | MM | ACC 9 | DSMZ | 15000 | not tested |
| RC-K8 | DLBCL | ACC 561 | DSMZ | 10000 | not tested |
| Pfeiffer | DLBCL | CRL-2632 | ATCC | 0 | 100000 |
| THP-1 | AML | ACC 16 | DSMZ | 400000 | 40000 |
| Oci-AML3 | AML | ACC 582 | DSMZ | 200000 | 40000 |
| monomac6 | AML | ACC 124 | DSMZ | 200000 | 30000 |
| KG-1 | AML | CCL-246 | ATCC | 180000 | 100000 |
| ML-2 | AML | ACC 15 | DSMZ | 150000 | 10000 |
| U937 | AML | CRL-1593.2 | ATCC | 130000 | not tested |
| Nomo-1 | AML | ACC 542 | DSMZ | 110000 | 30000 |
| MEGAL | AML | ACC 719 | DSMZ | 100000 | 110000 |
| AML-193 | AML | ACC 549 | DSMZ | 100000 | not tested |
| MOLM-13 | AML | ACC 554 | DSMZ | 90000 | 10000 |
| HL-60 | AML | CLL-240 | ATCC | 90000 | 10000 |
| Oci-M1 | AML | ACC 529 | DSMZ | 0 | 200000 |

ABCs = Antibodies Bound per Cell

The origins/sources of the cell lines are as follows:

| Cell line: | Source: |
|---|---|
| Daudi | ATCC; CCL-213 |
| Ramos | ATCC; CRL-1596 |
| Wien-133 | BioAnaLab, Oxford, U.K |
| NALM-16 | DSMZ; ACC 680 |
| U266 | ATCC; TIB-196 |
| RC-K8 | DSMZ; ACC 561 |

### Example 2—Binding of CD38 Antibodies and Variants Thereof to Human and Cynomolgus CD38 Expressed on the Cell Surface

Binding to cell surface expressed CD38 on Daudi and NALM16 cells and PBMCs from cynomolgus monkeys, was determined by flow cytometry. Cells, resuspended in RPMI containing 0.2% BSA, were seeded at 100,000 cells/well in polystyrene 96 well round-bottom plates (Greiner bio-one) and centrifuged for 3 minutes at 300×g, 4° C. Serial dilutions (0.005-10 μg/mL final antibody concentration in 3x serial dilutions) of CD38 or control antibodies were added and cells were incubated for 30 minutes at 4° C. Plates were washed/centrifuged twice using FACS buffer (PBS/0.1% BSA/0.01% Na-Azide). Next, cells were incubated for 30 minutes at 4° C. with R-Phycoerythrin (PE)-conjugated goat-anti-human IgG F(ab′)₂ (Jackson) diluted 1/100 in PBS/0.1% BSA/0.01% Na-Azide or FITC-conjugated goat-anti-human IgG (Southern Biotech) for analysis of cynomolgus PBMCs. Cells were washed/centrifuged twice using FACS buffer, resuspended in FACS buffer and analyzed by determining mean fluorescent intensities using a FACS_Fortessa (BD). Binding curves were generated using non-linear regression (sigmoidal dose-response with variable slope) analyses within GraphPad Prism V6.04 software (GraphPad Software).

FIG. 2 shows that CD38 antibodies IgG1-B, IgG1-C and IgG1-A bind dose-dependently to CD38 expressing NALM16 cells. Introduction of the hexamerization-enhancing E430G mutation into these antibodies did not affect the binding.

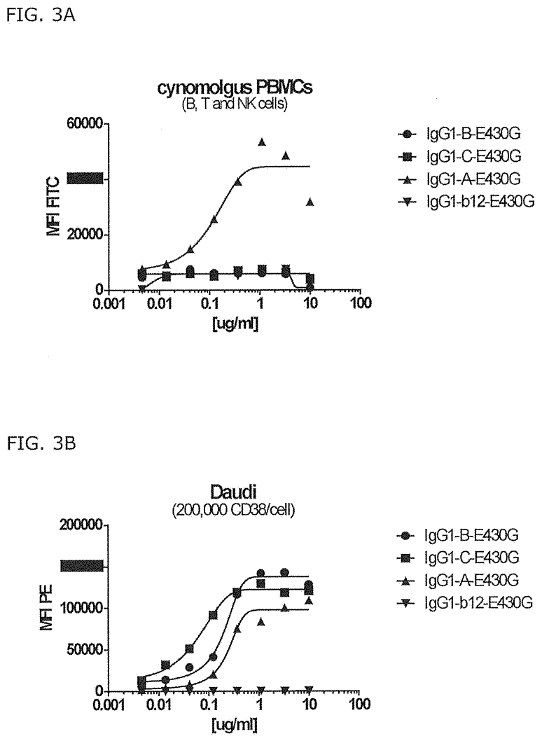

FIG. 3 shows that CD38 antibody IgG1-A-E430G, but not IgG1-B-E430G and IgG1-C-E430G, binds dose-dependently to CD38 expressed on cynomolgus PBMCs (A). The average binding to CD38 expressed on cynomolgus B, T and NK cells is depicted, gated based on FSC and SSC. As a positive control, binding to Daudi cells expressing high copy numbers of human CD38 is also depicted (B).

### Example 3—Complement-Dependent Cytotoxicity (CDC) by E430G-Mutated CD38 Antibodies

### CDC on Tumor Cell Lines

Daudi, Wien133, Ramos, NALM16, U266 and RC-K8 cells were resuspended in RPMI containing 0.2% BSA and plated into polystyrene 96-well round-bottom plates (Greiner bio-one) at a density of 1×10⁵ cells/well (40 μL/well). CD38 antibodies, variants thereof and isotype control Abs were serially diluted (0.0002-10 μg/mL final antibody concentration in 3x serial dilutions) and 40 μL of diluted Ab was added per well. Cells and Ab were pre-incubated for 20 minutes at room temperature after which, 20 μL of pooled normal human serum (Sanquin) was added to each well and incubated for another 45 minutes at 37° C. After that, plates were centrifuged (3 minutes, 1200 rpm) and supernatant was discarded. Cell pellets were resuspended in FACS-buffer supplemented with 0.25 μM topro-3 iodide (Life technologies), and lysis was detected by measuring the percentage of topro-3 iodine-positive cells on a FACS_Fortessa (BD). CDC was depicted as percent lysis. Data shown is N=3 (Daudi and NALM16), N=2 (Wien133 and U266 cells), or N=1 (RC-K8 and Ramos). Isotype control antibodies were only included on Daudi and Wien133 cells.

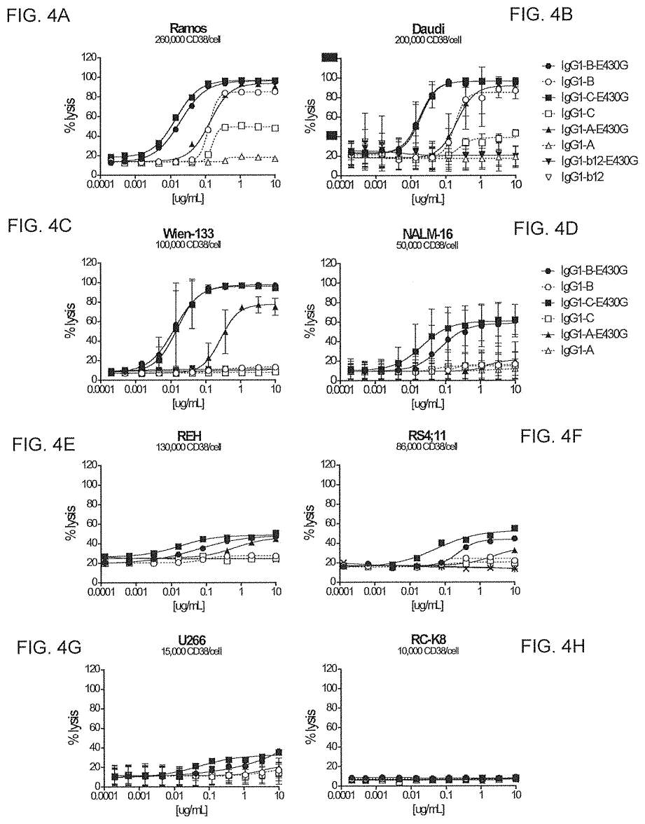

FIG. 4 demonstrates that CD38 antibodies B, C and A without the E430G mutation induce ˜85, ˜50 and 0 percent lysis of Ramos and Daudi cells. No significant lysis by these CD38 antibodies was seen for any of the other tested cell lines. Introduction of an E430G mutation in these CD38 antibodies resulted in higher CDC activity at significantly lower antibody concentration. All 3 antibodies with the E430G mutation induced up to 100% lysis of Ramos and Daudi cells. Moreover, on cell lines with lower CD38 expression, E430G-mutated CD38 antibodies were able to induce maximum (Wien133) or partial (NALM16 and U266) CDC, whereas CD38 antibodies without E430G-mutation did not induce CDC. These results demonstrate that CD38 Abs with an E430G mutation induce stronger CDC and require less CD38 expression compared to the CD38 antibodies without E430G mutation. In tumor cells with lower CD38 expression levels (NALM-16, RS4; 11, and REH), IgG1-C-E430G showed lower EC50 values compared to IgG1-B-E430G.

TABLE 3
EC50-values of lysis.
Some cell lines were tested only once (Ramos, RS4; 11, REH)

| | | | | | | RS4; | |
|---|---|---|---|---|---|---|---|
| | Ramos | Daudi | Wien-133 | NALM-16 | U266 | 11 | REH |
| B | 0.126 | 0.183 | 0.199 | — | — | — | — |
| B- | 0.019 | 0.018 | 0.013 | 0.075 | — | 0.243 | 0.054 |
| E430G | | | | | | | |
| C | 0.158 | 0.250 | 0.193 | — | — | — | — |
| C- | 0.014 | 0.019 | 0.015 | 0.022 | 0.052 | 0.056 | 0.017 |
| E430G | | | | | | | |
| A | — | — | — | — | — | — | — |
| A- | 0.133 | 0.206 | 0.271 | — | — | — | — |
| E430G | | | | | | | |

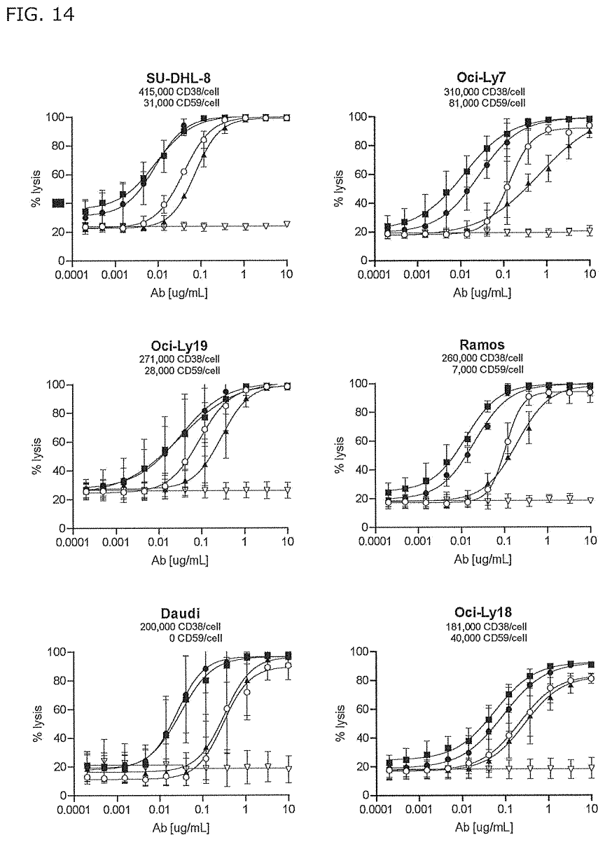

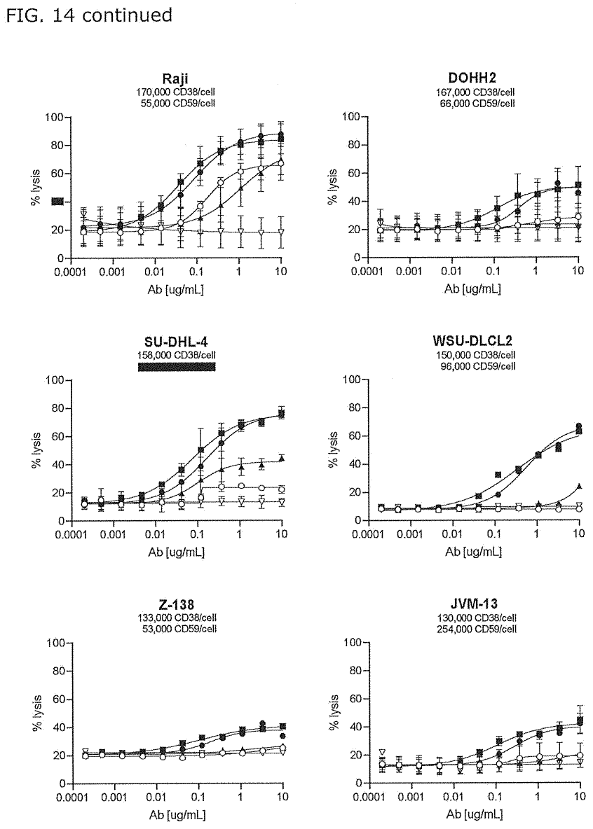

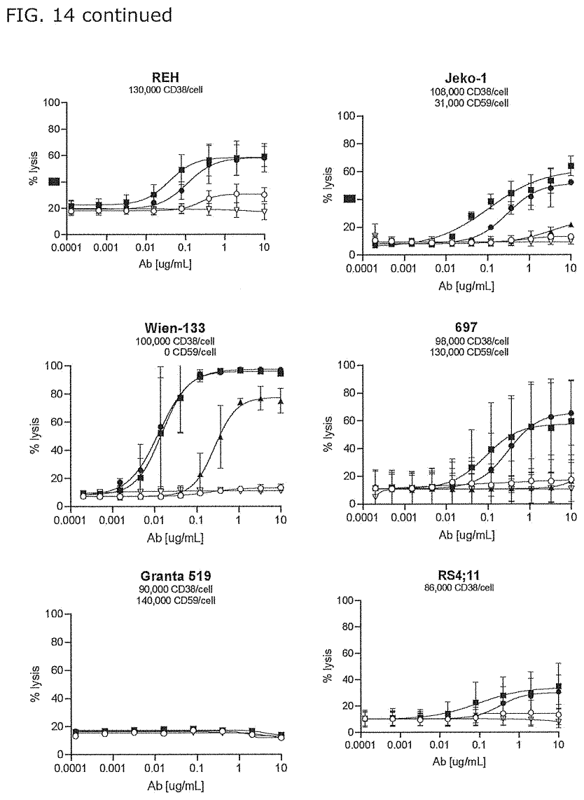

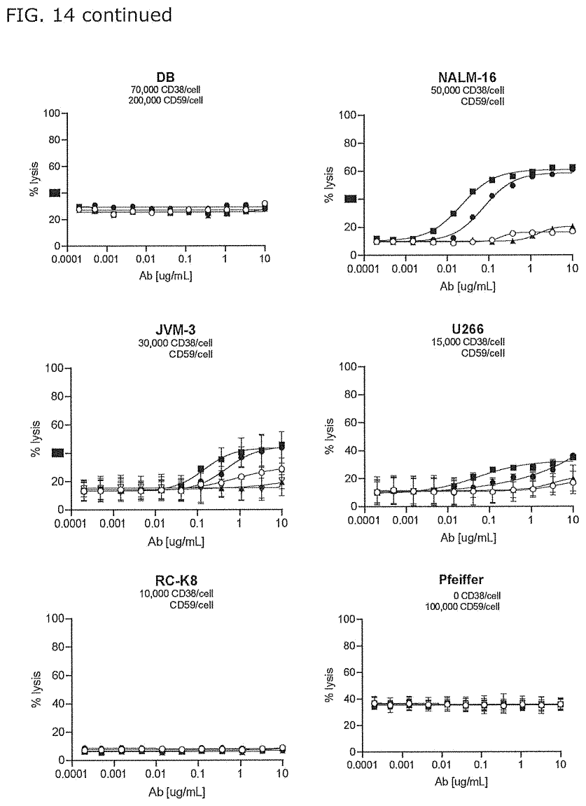

The above described CDC assay was repeated with a number of further tumor cell lines derived from B-cell tumors, including DLBCL, Burkitt's lymphoma, FL, MCL, B-ALL, CLL, or MM, and the antibodies IgG1-B, IgG1-B-E430G, IgG1-C-E430G, IgG1-A-E430G and isotype control antibody. The percentage lysis was plotted against the antibody concentration and maximum percent lysis and EC50 values were calculated using Graphpad Prism (GraphPad Software, Inc; version 8.1.0) software and shown in Table 4. The results are also shown in FIG. 14.

FIG. 14 demonstrates that wild type CD38 mAb IgG1-B induced lysis of high CD38 expressing cell lines; SU-DHL-8, Oci-Ly-7, Oci-Ly-19, Ramos, Daudi, Oci-Ly-18 and Raji, but not for any of the other cell lines that express less CD38 molecules on the membrane. Introduction of an E430G mutation in IgG1-B resulted in higher CDC activity at significantly lower Ab concentration on cell lines that were already sensitive to wild type IgG1-B and resulted in lysis of additional cell lines with lower CD38 copy number that were insensitive to IgG1-B induced CDC (e.g.: DOHH2, SU-DHL-4, WSU-DLCL2, Z-138, JVM-13, REH, Jeko-1, Wien-133, 697, RS4; 11, NALM-16 and JVM-3). Some cell lines with very low CD38 expression (RC-K8 and Pfeiffer) or very high CD59 expression (DB and Granta-519) showed no lysis upon exposure to IgG1-B and IgG1-B-E430G. On virtually all cell lines tested, IgG1-C-E430G induced cell lysis at a lower antibody concentration compared to IgG1-B-E430G, whereas IgG1-A-E430G induced lysis at much higher Ab concentrations. This is also reflected by the higher EC50 values for IgG1-A-E430G in Table 4. This demonstrates that E430G mutated CD38 mAbs induce stronger CDC compared to wild type CD38 antibodies and induce CDC on tumor cells with lower CD38 expression levels, in which wild type CD38 antibodies do not induce CDC. Moreover, the potency of E430G-mutated CD38 antibodies to induce CDC may vary between different CD38-targeting antibody clones.

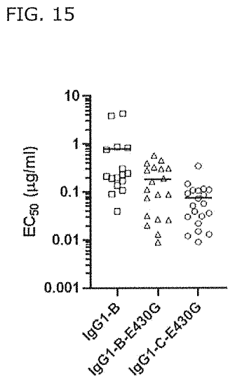

FIG. 15 shows a summary of some of the EC50 values depicted in Table 4. EC50 values of CDC induced by antibodies IgG1-B, IgG1-B-E430G and IgG1-C-E430G on 20 different B cell tumor cell lines are shown. Each square, triangle or circle represents a different B cell tumor cell line. EC50 values obtained with AML cell lines were not included because IgG1-B-E430G was not tested on AML cell lines.

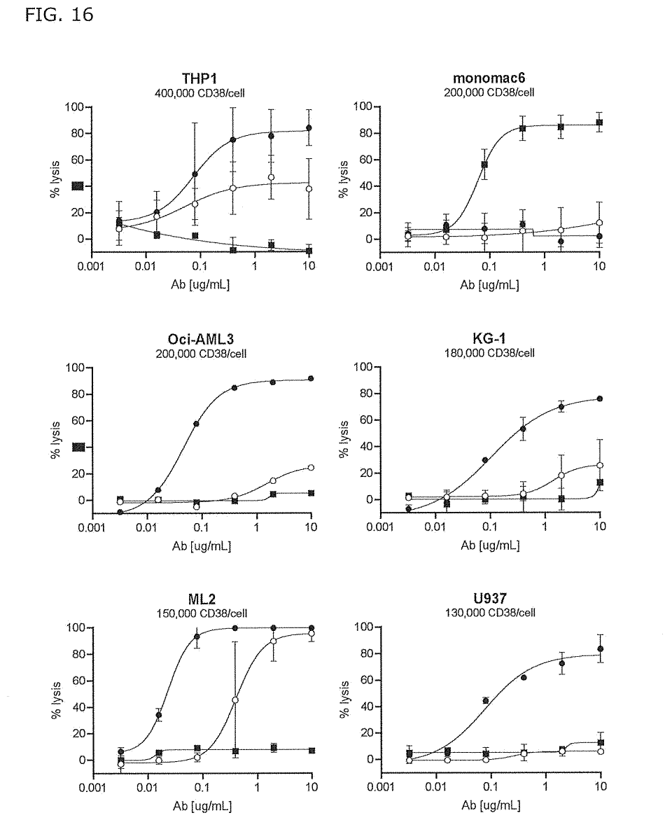

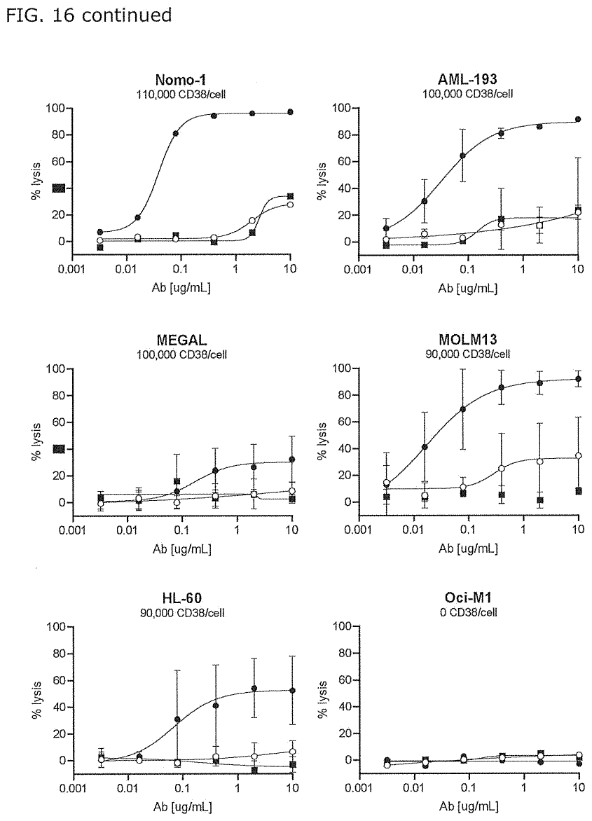

CDC by IgG1-C-E430G was also evaluated on a selection of Acute Myeloid Leukemia (AML) cell lines (FIG. 16). It was performed as described above for the B cell tumor cell lines with the only difference being the tumor cell line(s).

FIG. 16 demonstrates that CDC was induced by IgG1-C-E430G in all CD38 expressing AML cell lines, while no CDC was observed in CD38 negative AML cell lines. CDC by IgG1-C-E430G occurred at much lower EC50 value compared to IgG1-B, while the maximal cell lysis was higher for IgG1-C-E430G compared to IgG1-B (Table 4).

TABLE 4
maximum lysis and EC50 values of lysis

| | IgG1-C-E430G | | | IgG1-B | | | IgG1-B-E430G | | | |
|---|---|---|---|---|---|---|---|---|---|---|
| | EC50 | max % | min % | EC50 | max % | min % | EC50 | max % | min % | |
| cell line | ug/mL | lysis | lysis | ug/mL | lysis | lysis | ug/mL | lysis | lysis | N |
| SU-DHL-8 | 0.009 | 100.0 | 35.4 | 0.040 | 99.8 | 22.8 | 0.009 | 100.0 | 31.0 | 3 |
| Oci-Ly-7 | 0.012 | 99.2 | 21.0 | 0.138 | 91.7 | 18.1 | 0.027 | 98.9 | 19.4 | 3 |
| Oci-Ly-19 | 0.031 | 100.0 | 23.4 | 0.091 | 98.8 | 24.6 | 0.032 | 100.0 | 27.4 | 3 |
| Ramos | 0.013 | 99.5 | 25.0 | 0.108 | 94.0 | 17.1 | 0.020 | 99.3 | 19.4 | 3 |
| Daudi | 0.030 | 96.5 | 17.9 | 0.307 | 89.5 | 11.3 | 0.026 | 96.7 | 19.1 | 4 |
| Oci-Ly18 | 0.057 | 92.5 | 24.5 | 0.212 | 83.3 | 17.3 | 0.088 | 92.3 | 18.8 | 3 |
| Raji | 0.036 | 83.8 | 18.4 | 0.171 | 65.8 | 18.6 | 0.088 | 87.1 | 17.8 | 4 |
| DOHH2 | 0.115 | 50.3 | 19.2 | 0.874 | 29.4 | 19.4 | 0.399 | 49.7 | 20.9 | 3 |
| SU-DHL-4 | 0.073 | 75.5 | 12.0 | ND | 23.5 | 12.5 | 0.165 | 76.6 | 11.8 | 3 |
| WSU-DLCL2 | 0.345 | 65.9 | 6.3 | ND | 7.8 | 8.3 | 0.577 | 67.6 | 7.7 | 1 |
| Z-138 | 0.106 | 41.2 | 20.6 | 4.327 | 28.1 | 19.5 | 0.190 | 38.0 | 21.2 | 1 |
| JVM-13 | 0.146 | 43.6 | 13.3 | 0.769 | 30.5 | 13.3 | 0.458 | 44.5 | 13.3 | 3 |
| REH | 0.039 | 58.3 | 22.4 | 0.232 | 30.6 | 18.0 | 0.112 | 58.1 | 19.2 | 3 |
| Jeko-1 | 0.108 | 61.6 | 5.5 | 0.833 | 13.2 | 9.3 | 0.302 | 51.5 | 8.1 | 2 |
| Wien133 | 0.015 | 96.0 | 8.2 | 0.199 | 13.2 | 7.0 | 0.013 | 97.4 | 7.9 | 2 |
| 697 | 0.087 | 57.6 | 10.9 | ND | ND | ND | 0.308 | 65.6 | 11.1 | 3 |
| Granta-519 | ND | 17.4 | 13.5 | ND | 15.5 | 77.8 | ND | 16.7 | 13.1 | 3 |
| RS4; 11 | 0.093 | 33.9 | 9.8 | ND | 14.1 | 9.9 | 0.328 | 29.9 | 10.1 | 3 |
| DB | ND | ND | ND | ND | ND | ND | ND | ND | ND | 1 |
| NALM-16 | 0.022 | 60.9 | 10.1 | 0.193 | 16.2 | 9.4 | 0.075 | 58.6 | 9.7 | 3 |
| JVM-3 | 0.110 | 42.5 | 11.6 | 0.245 | 19.0 | 12.8 | 0.287 | 40.2 | 12.2 | 2 |
| U266 | 0.052 | 32.5 | 9.6 | 3.889 | 19.1 | 10.8 | ND | ND | 8.7 | 2 |
| RC-K8 | ND | ND | 6.6 | ND | 7.7 | ND | ND | 8.2 | 8.6 | 1 |
| Pfeiffer | ND | ND | ND | ND | ND | ND | ND | ND | ND | 2 |
| THP-1 | 0.075 | 81.5 | 12.6 | 0.051 | 42.4 | 6.1 | NT | NT | NT | 3 |
| Oci-AML3 | 0.046 | 90.4 | 0.0 | 1.485 | 26.1 | 0.0 | NT | NT | NT | 3 |
| monomac6 | 0.093 | 83.9 | 14.0 | 0.053 | 54.1 | 0.0 | NT | NT | NT | 3 |
| KG-1 | 0.104 | 77.7 | 0.0 | 1.401 | 26.2 | 2.0 | NT | NT | NT | 3 |
| ML-2 | 0.023 | 99.6 | 5.1 | 0.414 | 95.4 | 0.0 | NT | NT | NT | 3 |
| U937 | 0.057 | 68.6 | 0.0 | 0.140 | 34.3 | 0.0 | NT | NT | NT | 2 |
| Nomo-1 | 0.039 | 95.9 | 6.7 | 1.937 | 28.4 | 1.9 | NT | NT | NT | 3 |
| MEGAL | 0.170 | 30.1 | 0.7 | ND | ND | ND | NT | NT | NT | 3 |
| AML-193 | 0.032 | 89.5 | 0.8 | ND | ND | ND | NT | NT | NT | 2 |
| MOLM-13 | 0.017 | 92.0 | 0.0 | 0.290 | 32.5 | 9.8 | NT | NT | NT | 3 |
| HL-60 | 0.070 | 52.5 | 0.0 | 4.519 | 10.2 | 0.0 | NT | NT | NT | 3 |
| Oci-M1 | ND | ND | ND | ND | ND | ND | NT | NT | NT | 1 |

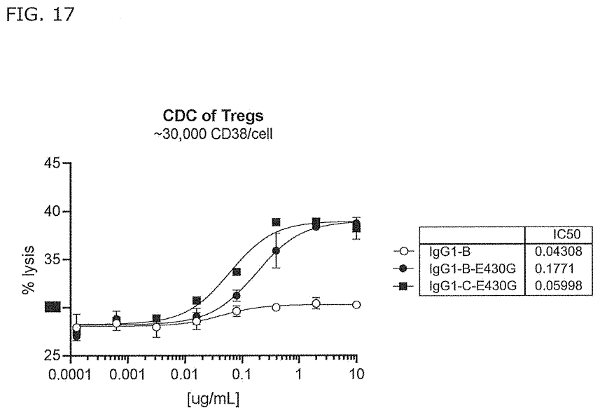

Induction of CDC by wild type and E430G mutated CD38 antibodies using T regulatory cells was also determined. The T regulatory cells were generated as described in Example 8 (Trogocytosis of CD38 from T regulatory cells) and tested in a CDC assay as described above for the tumor cell lines. The percentage of lysis is shown in FIG. 17 together with the EC50 values.

FIG. 17 demonstrates that IgG1-B induced virtually no lysis of T regulatory cells; while IgG1-B-E430G and IgG1-C-E430G induced lysis of T regulatory cells, where IgG1-C-E430G showed a lower EC50 value compared to IgG1-B-E430G.

### CDC in Whole Blood

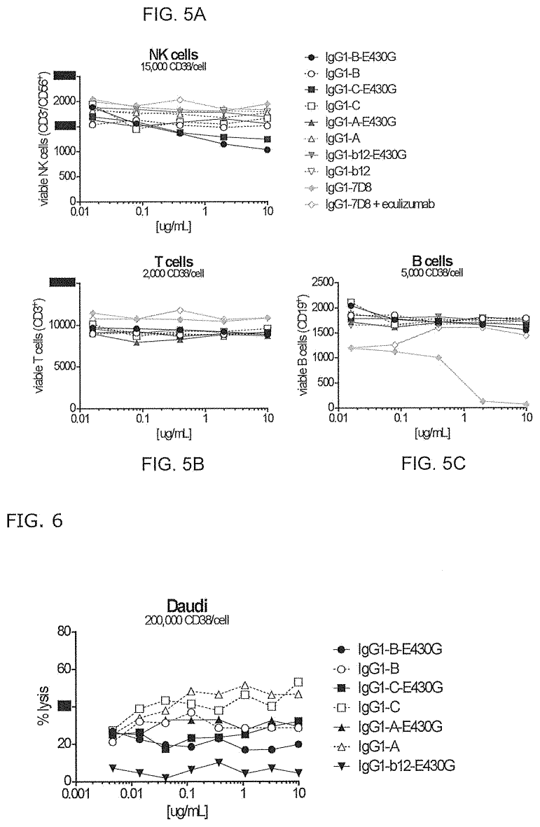

Whole blood from a healthy donor was collected in hirudin tubes to prevent coagulation without interference with physiological calcium levels (required for CDC). 50 μL/well was plated into 96-well flat-bottom tissue culture plates (Greiner bio-one). CD38 antibodies, variants thereof and control Abs were serially diluted in RPMI containing 0.2% BSA (0.016-10 μg/mL final antibody concentration in 5x serial dilutions) and 50 μL of diluted Ab was added per well and incubated overnight at 37° C. As a positive control for CDC on B cells, the CD20 Ab IgG1-7D8 was tested with and without 60 μg/mL eculizumab to block CDC. Cells were transferred to polystyrene 96-well round-bottom plates (Greiner bio-one, centrifuged), centrifuged (3 minutes, 1200 rpm) and washed once with 150 μL PBS (B. Braun) per well. Cell pellets were resuspended in 80 μL PBS with 1000x diluted amine reactive viability dye (BD) and incubated 30 minutes at 4° C. Next, cells were washed with 150 μL PBS and incubated with 80 μL PBS containing a cocktail of lymphocyte phenotyping antibodies (1:200 mouse anti-human CD3-EF450 [OKT3, ebioscience], 1:50 mouse anti-human CD19-BV711 [HIB19, Biolegend] and 1:100 mouse anti-human CD56-PE/CF594 [NCAM16.2, BD]) for 30 minutes at 4° C. Cells were washed with 150 μL PBS and incubated 10 minutes at 4° C. with 150 μL erythrocyte lysis solution (10 mM KHCO3 [Sigma], 0.01 mM EDTA [Fluka], 155 mM NH4Cl [Sigma] dissolved in 1 L of H₂O [B. Braun] and adjusted to pH 7.2). Cells were washed with 150 μL FACS buffer, re-suspended in 100 μL FACS buffer and analyzed on a FACS_Fortessa (BD). The number of viable NK cells (CD56^{pos}, CD3^{neg} and amine reactive viability dye^{neg}), T cells (CD3^{pos} and amine reactive viability dye^{neg}) and B cells (CD19^{pos} and amine reactive viability dye^{neg}) is depicted in FIG. 5. Data is shown from 1 representative donor out of 5 tested.

FIG. 5 demonstrates that CD38 antibodies containing the E430G mutation induce minimal CDC of healthy blood lymphocytes. The positive control CD20 Ab IgG1-7D8 demonstrated specific CDC of CD20-positive B cells, which was completely blocked by the CDC inhibitor eculizumab. Wild type IgG1 CD38 antibodies did not induce CDC of B, T and NK cells. Some CDC was observed for NK cells after incubation with clones B and C containing the E430G mutation (approximately 40% NK cell lysis at the highest concentration with IgG1-B-E430G), but not B and T cells.

Overall, these results indicate that E430G mutated CD38 antibodies have broad CDC activity against a panel of tumor cell lines with variable CD38 expression. CD38 antibodies with an E430G mutation were also tested against lymphocytes obtained from healthy donors, and were shown to only induce up to 40% lysis of NK cells. NK cells express on average 15,000 CD38/cell which is similar to the MM cell line U266. Both cell types are equally sensitive to CDC by E430G mutated CD38 antibodies, indicating that CDC by E430G mutated CD38 antibodies is correlated to CD38 expression. Without being limited to theory, based on these data, it is believed that the threshold for CDC by E430G-mutated CD38 antibodies lays around 15,000 CD38 molecules/cell. While most B cell tumor cell lines express higher levels of CD38 ranging from 15,000-400,000 CD38 molecules/cell, healthy lymphocytes express only 2,000-15,000 CD38 molecules/cell which makes these cells less vulnerable to CDC by E430G mutated CD38 antibodies.

### Example 4—Antibody-Dependent Cellular Cytotoxicity (ADCC) by E430G-Mutated CD38 Antibodies

The capacity of E430G mutated CD38 antibodies to induce antibody-dependent cellular cytotoxicity (ADCC) was determined by a chromium release assay. Daudi cells were collected (5×10⁶cells/mL) in 2 mL culture medium (RPMI 1640 supplemented with 0.2% BSA), to which 100 uCi ⁵¹Cr (Chromium-51; PerkinElmer) was added. Cells were incubated in a water bath at 37° C. for 1 hour while shaking. After washing of the cells (twice in PBS, 1500 rpm, 5 min), the cells were resuspended in culture medium and counted by trypan blue exclusion. Cells were diluted to a density of 1×10⁵ cells/mL and pipetted into 96-well round-bottom microtiter plates (Greiner Bio-One), and 50 μL of a concentration series of (0.005-10 μg/mL final concentrations in 3-fold dilutions) CD38 or isotype control antibody, diluted in culture medium was added. Cells were pre-incubated with Ab at room temperature (RT) for 15 min.

Meantime, peripheral blood mononuclear cells (PBMCs) from healthy volunteers (Sanquin) were isolated from 45 mL of freshly drawn heparin blood (buffy coats) using lymphocyte separation medium (Bio Whittaker) according to the manufacturer's instructions. After resuspension of cells in culture medium, cells were counted by trypan blue exclusion and diluted to a density of 1×10⁷ cells/mL.

After the pre-incubation of target cells with Ab, 50 μL effector cells was added, resulting in an effector to target cell ratio of 100:1. Cells were incubated for 4 hours at 37° C. and 5% CO₂. For determination of maximal lysis, 50 μL ⁵¹Cr-labeled Daudi cells (5,000 cells) were incubated with 100 μL 5% Triton-X100; for determination of spontaneous lysis (background lysis), 5,000 ⁵¹Cr-labeled Daudi cells were incubated in 150 μL medium without any antibody or effector cells. The level of antibody-independent cell lysis was determined by incubating 5,000 Daudi cells with 500,000 PBMCs without antibody. Plates were centrifuged (1200 rpm, 10 min) and 75 μL of supernatant was transferred to micronic tubes, after which the released ⁵¹Cr was counted using a gamma counter. The percentage of antibody-mediated lysis was calculated as follows:

% specific lysis=(cpm sample−cpm spontaneous lysis)/(cpm maximal lysis−cpm spontaneous lysis) wherein cpm is counts per minute.

FIG. 6 shows that all CD38 Abs were able to induce lysis of Daudi, as indicated by the increased lysis that was seen for CD38 Abs in comparison to the isotype control (IgG1-b12-E430G). Already at the lowest antibody concentration cell lysis was noted, suggesting that antibodies should have been further diluted in order to observe a dose-dependent effect. CD38 antibodies that contain an E430G mutation showed lower maximum lysis compared to wild type antibodies.

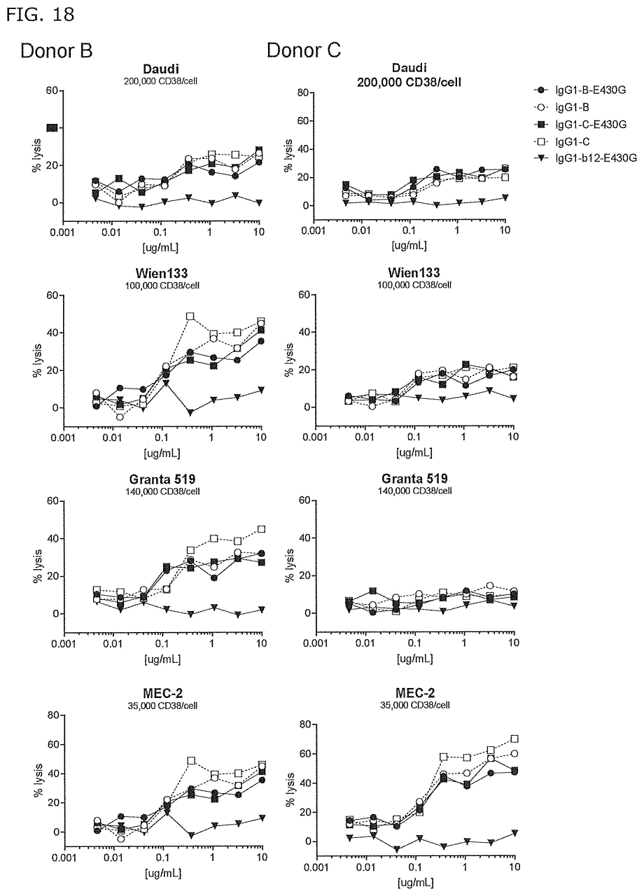

The above chromium release assay was repeated with peripheral blood mononuclear cells from different healthy volunteers (effector cells), the following target cells: Daudi, Wien-133, Granta 519 and MEC-2, and with the antibodies IgG1-B-E430G, IgG1-B, IgG1-C-E430G, IgG1-C and IgG1-b12-E430G. The results are shown in FIG. 18.

FIG. 18 shows that all CD38 Abs were able to induce lysis of Daudi, Wien-133, Granta 519 and MEC-2 cells as indicated by the increased lysis that was seen for CD38 Abs in comparison to the isotype control (IgG1-b12-E430G). In most instances dose-dependent target cell lysis was seen, but some variation was observed between different PBMC donors.

The ability of CD38 antibodies to induce ADCC was further evaluated using a luminescent ADCC reporter bioassay (Promega, Cat # G7018) that detects FcγRIIIa (CD16) crosslinking, as a surrogate for ADCC. As effector cells, the kit provides Jurkat human T cells that are engineered to stably express high affinity FcγRIIIa (V158) and a nuclear factor of activated T cells (NFAT)-response element driving expression of firefly luciferase. Briefly, Daudi or T regulatory cells (5,000 cells/well) were seeded in 384-well white Optiplates (Perkin Elmer) in ADCC Assay Buffer [RPMI-1640 medium [(Lonza, Cat # BE12-115F) supplemented with 3.5% Low IgG Serum] and incubated for 6 hours at 37° C./5% CO2 in a total volume of 30 μL containing antibody concentration series (0.5-250 ng/mL final concentrations in 3.5-fold dilutions) and thawed ADCC Bioassay Effector Cells. After adjusting the plates for 15 minutes to room temperature (RT), 30 μL Bio Glo Assay Luciferase Reagent was added and plates were incubated for 5 minutes at RT. Luciferase production was quantified by luminescence readout on an EnVision Multilabel Reader (Perkin Elmer). Background levels were determined from wells to which only target cells and antibody (no effector cells) was added. As negative control, wells containing only target and effector cells (no antibody) were used.

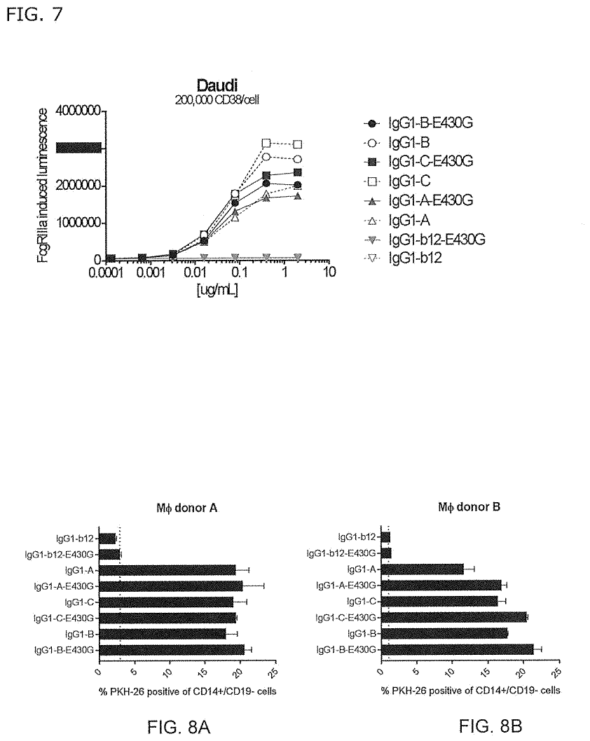

FIG. 7 shows the results obtained with the Daudi cells, which show that CD38 antibodies were highly effective in inducing dose-dependent FcγRIIIa cross-linking as determined in the reporter assay. CD38 antibodies that contained an E430G mutation showed lower maximum cross-linking compared to the respective wild type antibodies, which was in line with results obtained for the chromium release assay.

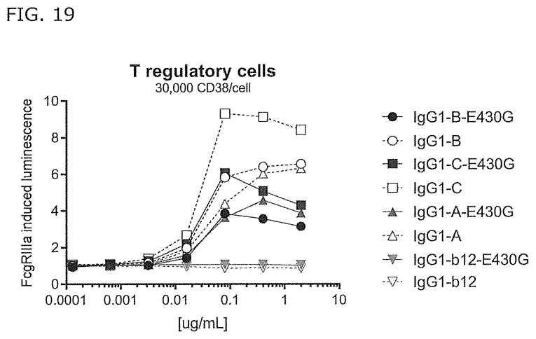

FIG. 19 shows the results obtained with the T regulatory cells, which show that CD38 antibodies were highly effective in inducing dose-dependent FcγRIIIa cross-linking as determined in the reporter assay. CD38 antibodies that contained an E430G mutation showed lower maximum cross-linking compared to the respective wild type antibodies.

### Example 5—Antibody-Dependent Cellular Phagocytosis (ADCP) by E430G-Mutated CD38 Antibodies

The capacity of E430G mutated CD38 antibodies to induce antibody-dependent cellular phagocytosis was adapted from Overdijk M. B. et al. mAbs 7:2,311-320. Macrophages were obtained by isolating PBMCs from healthy volunteers (Sanquin) using lymphocyte separation medium (Bio Whittaker) according to manufacturer's instructions. From the PBMCs, monocytes were isolated via negative selection, using Dynabeads Untouched Human Monocyte isolation kit (Invitrogen). The isolated monocytes were cultured 3 days in serum-free dendritic cell medium (CellGenix Gmbh) supplemented with 50 ng/mL GM-CSF (Invitrogen), followed by 2 days in serum-free dendritic cell medium supplemented with 100 ng/mL GM-CSF, to induce macrophage differentiation. The differentiated macrophages were detached using versene (Life Technologies) and cell scraping and characterized by flow cytometry for staining with CD1a-FITC (BD), CD14-PE/Cy7 (BD), CD40-APC/H7 (BD), CD80-APC (Miltenyi biotec), CD83-PE (BD) and CD86-PerCP-Cy5.5 (Biolegend). Macrophages were seeded at 100,000 cells per well into 96-well flat-bottom culture plates (Greiner bio-one) and allowed to adhere overnight at 37° C. in serum-free dendritic cell medium supplemented with 100 ng/mL GM-CSF.

Target cells (Daudi) were labeled with PKH-26 (Sigma) according to manufacturer's instructions, opsonized with 10 μg/mL CD38 antibody (30 minutes at 4° C.), washed three times with FACS buffer and added to the macrophages at an effector:target (E:T) ratio of 5:1. The plate was briefly spinned at 300 rpm to bring the effector cells and target cells in close proximity and incubated 45 minutes at 37° C. Next, macrophages were collected using versene and stained with CD14-BV605 (biolegend) and CD19-BV711 (biolegend). Phagocytosis was depicted as the percentage of CD14-positive macrophages that were also positive for PKH-26, but negative for CD19 (to exclude macrophages that are only attached to Daudi cells), measured on a flow cytometer (BD).

FIG. 8 shows that all CD38 Abs were able to induce ADCP of Daudi cells, as indicated by the increased percentage of PKH-29^{pos}, CD14^{pos} and CD19^{neg} macrophages that was seen for CD38 Abs in comparison to the isotype controls (IgG1-b12 and IgG1-b12-E430G). Depending on the donor used, CD38 antibodies that contain an E430G mutation showed a higher percentage of PKH-29^{pos}, CD14^{pos} and CD19^{neg} macrophages compared to wild type antibodies, indicating CD38-Ab mediated phagocytosis can be increased by introducing the E430G mutation.

### Example 6—Induction of Apoptosis by CD38 Antibodies on Tumor Cell Lines

Apoptosis induction by CD38 antibodies was investigated by overnight incubation of tumor cell lines with CD38 antibody followed by live/dead analysis on a flow cytometer. Cells, resuspended in RPMI containing 0.2% BSA, were seeded at 100,000 cells/well in 96 well flat-bottom tissue culture plates (Greiner bio-one). Serial dilutions (0.01-10 μg/mL final antibody concentration in 4x serial dilutions) of CD38 or control antibodies were added in the absence or presence of 10 μg/mL goat-anti-human IgG1 (Jackson) to provide additional Fc-cross-linking. Cells were incubated overnight at 37° C., washed/centrifuged twice using FACS buffer (PBS/0.1% BSA/0.01% Na-Azide), and resuspended in FACS buffer supplemented with 1:4000 diluted Topro-3-iodine (Life Technologies). Cell viability was analyzed on a FACS_Fortessa (BD) and depicted as the percentage of apoptotic (topro-3-iodine positive) cells.

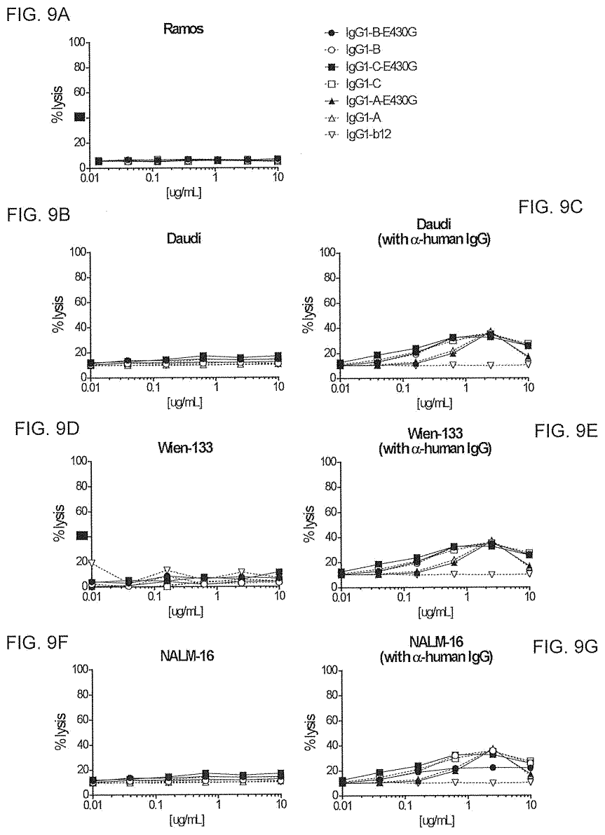

FIG. 9 shows that wild type and E430G mutated CD38 antibodies did not induce apoptosis alone, but the addition of an Fc-cross-linking antibody resulted in approximately 30% of apoptosis. No difference was seen between wild type and E430G mutated CD38 antibodies.

### Example 7—Inhibition of CD38 Enzyme Activity in the Absence of PBMCs

### Inhibition of CD38 Cyclase Activity

CD38 is an ecto-enzyme that converts NAD into cADPR and ADPR. These activities are dependent on the presence of H₂O. When H₂O is present, NAD is converted into ADPR, (glycohydrolase activity) and cADPR is converted into ADPR (hydrolase activity). About 95% of NAD is converted into ADPR through (glyco)hydrolase activity. In the absence of H₂O, CD38 turns NAD into cADPR using its cyclase activity. To measure inhibition of CD38 enzyme activity, NAD derivatives were used that become fluorescent after being processed by CD38.

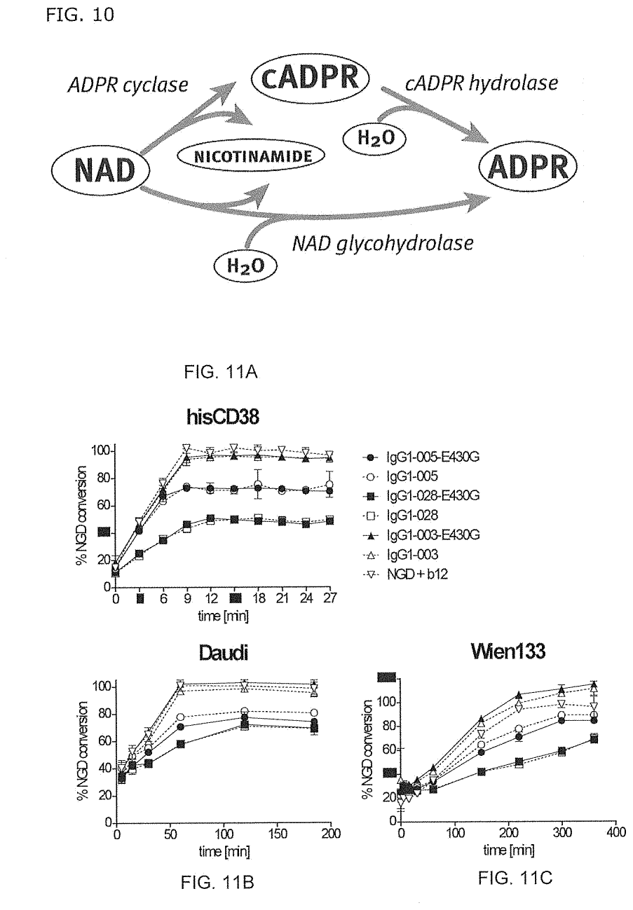

FIG. 10 illustrates the enzyme activities of CD38.

First, inhibition of CD38 cyclase activity was measured using nicotinamide guanine dinucleotide sodium salt phosphodiesterase (NGD, Sigma) as a substrate for CD38. As a source of CD38, tumor cell lines with different CD38 expression levels were used as well as recombinant his-tagged extracellular domain of CD38 (hisCD38). Tumor cells (Daudi and Wien133) were harvested and washed with 20 mM Tris-HCL. Cells were resuspended in 20 mM Tris-HCL and 200,000 cells/well were seeded in 96-well white opaque plates (PerkinElmer) in 100 μL/well. HisCD38 was seeded at 0.6 μg/mL in 100 μL/well 20 mM Tris-HCL. CD38 antibodies were diluted to 100 μg/mL in 20 mM Tris-HCL and 10 μL was added to the cells and hisCD38 (final concentration is 9 μg/mL) and incubated for 20 minutes at room temperature. Control wells were incubated with b12 antibody instead of CD38 antibody, or with no antibody at all. Next, 10 μL (80 μM) NGD diluted in 20 mM Tris-HCL was added to the plate and fluorescence was immediately measured on the Envision multilabel reader (PerkinElmer) using excitation 340 nm and emission 430 nm. The conversion of NGD was followed real time, by measuring fluorescence at the indicated time points in FIG. 11 until a plateau is reached. For hisCD38, fluorescence was measured every 3 minutes for 27 minutes, for Daudi cells fluorescence was measured after 5, 15, 30, 60, 120 and 185 minutes and for Wien133, fluorescence was measured after 5, 15, 30, 60, 150, 220, 300 and 360 minutes. Inhibition of CD38 cyclase activity was depicted as percent inhibition compared to control, where control is a sample with hisCD38 and NGD, but no Ab. One representative experiment is depicted for each condition tested.

FIG. 11A demonstrates that NGD was rapidly converted through hisCD38 cyclase activity. The conversion was complete after approximately 9 minutes. In the presence of CD38 Ab B the maximum percent of NGD conversion was reduced with ˜25%, in the presence of CD38 Ab C the maximum percent of NGD conversion was reduced with ˜50%, while CD38 Ab A had no effect on the total turnover of NGD. The inhibition of CD38 cyclase activity was not affected by presence of the E430G mutation. Similar results were seen in FIGS. 11B and 11C, where NGD conversion by CD38 present on Daudi and Wien133 cells were measured. The kinetics of NGD conversion were a bit slower on Daudi and especially Wien133 cells, which is likely correlated to less CD38 molecules being present. Nevertheless, 25% inhibition of CD38 cyclase activity was induced by Ab B (˜25% inhibition) and ˜40% inhibition of CD38 cyclase activity was induced by Ab C, while Ab A showed no effect. Wild type antibodies and E430G mutated antibodies showed the similar results, indicating that the E430G mutation does not impact antibody-mediated inhibition of CD38 cyclase activity.

### Example 8—Antibody-Dependent Trogocytosis by E430G Mutated CD38 Antibodies

### Trogocytosis by E430G Mutated CD38 Antibodies on Daudi Cells:

The capacity of E430G mutated CD38 antibodies to induce trogocytosis on Daudi cells was evaluated. Macrophages were obtained by isolating PBMCs from healthy volunteers (Sanquin) using lymphocyte separation medium (Bio Whittaker) according to manufacturer's instructions. From the PBMCs, monocytes were isolated via negative selection, using Dynabeads Untouched Human Monocyte isolation kit (Invitrogen). The isolated monocytes were cultured 3 days in serum-free dendritic cell medium (CellGenix Gmbh) supplemented with 50 ng/mL GM-CSF (Invitrogen), followed by 2 days in serum-free dendritic cell medium supplemented with 100 ng/mL GM-CSF, to induce macrophage differentiation. The differentiated macrophages were detached using versene (Life Technologies) and cell scraping and characterized by flow cytometry for staining with CD1a-FITC (BD), CD14-PE/Cy7 (BD), CD40-APC/H7 (BD), CD80-APC (Miltenyi biotec), CD83-PE (BD) and CD86-PerCP-Cy5.5 (Biolegend). Macrophages were seeded at 100,000 cells per well into 96-well flat-bottom culture plates (Greiner bio-one) and allowed to adhere overnight at 37° C. in serum-free dendritic cell medium supplemented with 100 ng/mL GM-CSF.

Target cells (Daudi) were labeled with PKH-26 (Sigma) according to manufacturer's instructions, opsonized with 10 μg/mL CD38 antibody (30 minutes at 4° C.), washed three times with FACS buffer and added to the macrophages at an effector:target (E:T) ratio of 5:1. The plate was briefly spinned at 300 rpm to bring the effector cells and target cells in close proximity and incubated 45 minutes at 37° C.

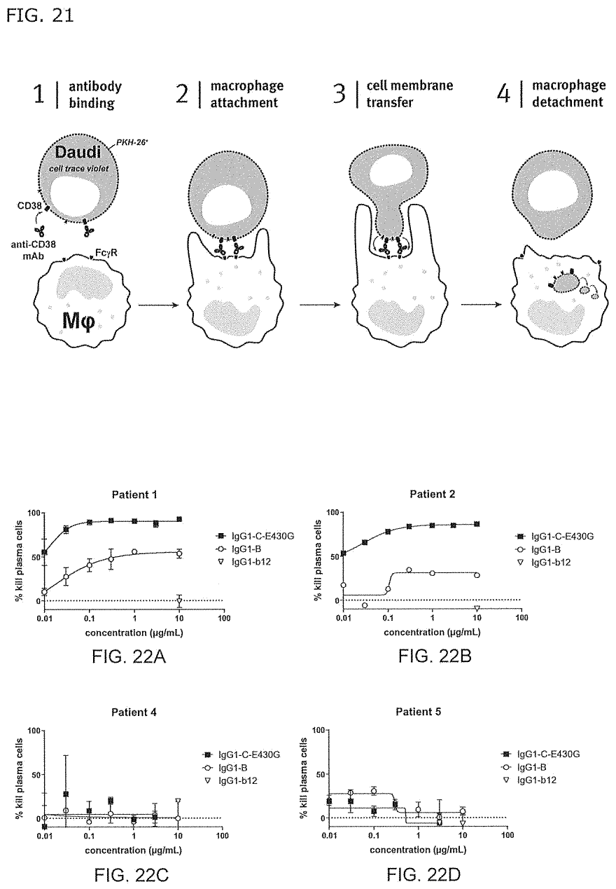

FIG. 21 illustrates the assay set-up used to measure trogocytosis.

CD38 expression and human IgG staining were determined on Daudi cells by incubation with FITC-conjugated CD38 clone A and goat anti-human IgG-FITC (Southern Biotech) respectively. CD38 clone A was used to stain CD38 because this Ab recognizes a non-overlapping epitope on CD38 compared to clones B and C.

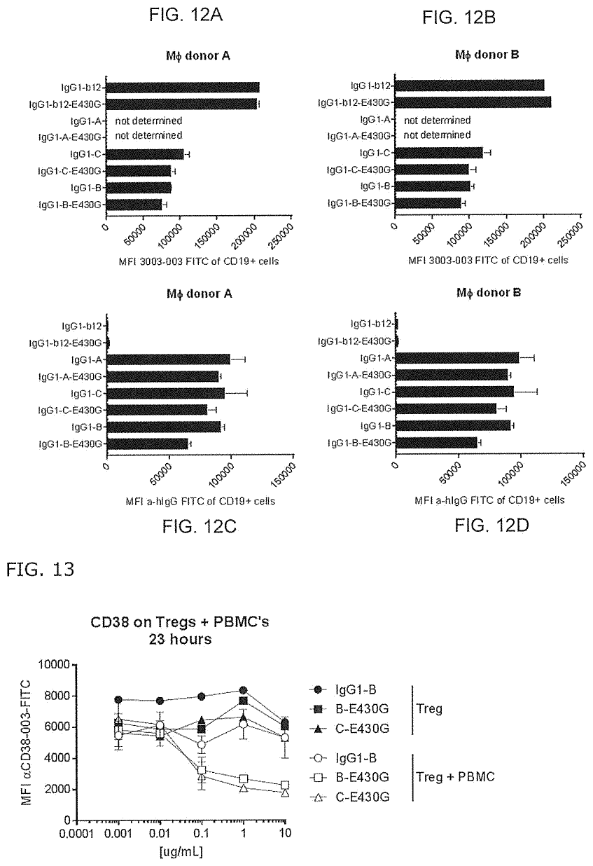

FIG. 12 shows that CD38 expression on Daudi cells was significantly reduced after 45 minute co-culture with macrophages and CD38 antibodies. The reduction in CD38 expression was strongest with E430G mutated CD38 antibodies. The same trend was seen for human IgG staining on antibody opsonized Daudi cells.

### Trogocytosis by E430G Mutated CD38 Antibodies on T Regulatory Cells:

T regulatory cells (Tregs) with high CD38 expression are more immune suppressive compared to Tregs with intermediate CD38 expression (Krejcik J. et al. Blood 2016 128:384-394). Therefore strategies to reduce CD38 expression on Tregs might reduce the immune suppressive effects of these cells. We investigated if E430G mutated CD38 antibodies can reduce CD38 expression on Tregs through trogocytosis. Tregs were isolated from PBMCs from healthy volunteers (Sanquin) using lymphocyte separation medium (Bio Whittaker) according to manufacturer's instructions. From the PBMCs, CD4⁺ T cells were isolated via negative selection, followed by enrichment for CD4⁺CD25⁺ T regulatory cells, using Treg isolation kit (Miltenyi) according to manufacturer's instructions. Subsequently, Tregs were expanded at 5×10⁴ cells/mL in serum-free dendritic cell medium supplemented with 5% human serum (Sigma), 1000 U/mL IL-2 (peprotech), 100 ng/mL rapamycin (Sigma) and CD3/CD28 coated beads (Gibco) at a bead:cell ratio of 4:1 for 20 days at 37° C. Every 3 to 4 days the cell density was adjusted to 5×10⁵ cells/mL using serum-free dendritic cell medium supplemented with 1000 U/mL IL-2 and 100 ng/mL rapamycin. T regulatory phenotype was followed over time using flow cytometry staining with the following antibodies: CCR7-BV785 (Biolegend), CD62L-FITC (BD), CD4-APC/efluor780 (e-biosciences), CD25-PerCP/Cy5 (Biolegend), Foxp3-PE/CF594 (BD), CTLA4-efluor660 (e-biosciences), CD127-PE/CY7 and CD38-GV605 (Biolegend).

To evaluate Ab induced trogocytosis of CD38 from Tregs, Tregs (target cells) were co-cultured with PBMCs (effector cells) and CD38 expression was monitored on the Tregs. In brief: PBMCs were isolated from buffy coats (Sanquin) using lymphocyte separation medium (Bio Whittaker) according to manufacturer's instructions and seeded in RPMI-1640 medium (Lonza) supplemented with 0.2% BSA at a density of 5×10⁵ cells per well and cultured 3 days to allow monocytes to adhere. Tregs were labeled with 0.25 μM CellTrace far red (CTFR) according to manufacturer's instruction and pre-incubated with E430G mutated CD38 Ab for 10 minutes at 37° C. Tregs were washed and 1×10⁵ Ab-opsonized cells per well were transferred to the plate with PBMCs. The PBMCs and Tregs were briefly spinned at 300 rpm to bring the cells in close proximity and incubated for 23 hours at 37° C. Trogocytosis of CD38 was measured by analyzing CD38 expression with FITC-conjugated CD38 clone A on CTFR-positive Tregs with flow cytometry.

FIG. 13 shows that CD38 expression on T regulatory cells was reduced after incubation with E430G mutated CD38 antibodies and PBMCs. Without PBMCs, no reduction of CD38 expression on T regulatory cells was seen, strongly suggesting trogocytosis. Furthermore, in presence of PBMCs, IgG1-B did not induce trogocytosis of CD38, while a strong reduction in CD38 expression was induced by E430G mutated B and C. This suggests that E430G mutated CD38 antibodies induce enhanced trogocytosis of CD38.

### Example 9: Anti-Tumor Activity of a E430G Mutated CD38 Antibody C in Patient Derived Diffuse Large B Cell Lymphoma Models

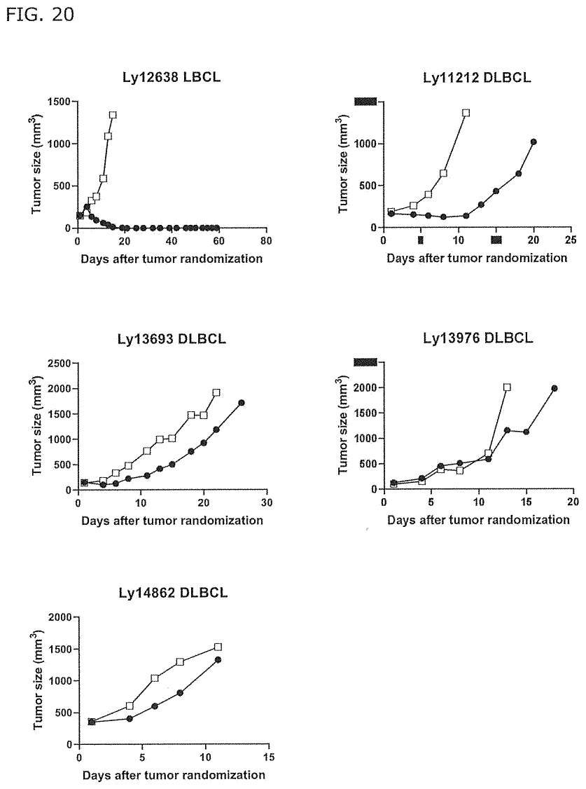

Patient derived Diffuse Large B Cell Lymphoma (DLBCL) cells were inoculated subcutaneous in CB17.SCID mice and antibody treatment (2 weekly doses of 5 mg/kg IgG1-C-E430G, injected intravenously; PBS was used as negative control) was initiated when tumors reached a mean volume of approximately 150-250 mm³. Tumor volumes were measured in two dimensions using a caliper, and the volume was expressed in mm³ using the formula: V=(L×W×W)/2, where V is tumor volume, L is tumor length (the longest tumor dimension) and W is tumor width (the longest tumor dimension perpendicular to L), and depicted over time in FIG. 20. Each treatment group consists of a single mouse. To calculate a response value the following formula was used; (tumor volume of IgG1-C-E430G treated mouse on day X−tumor volume of IgG1-C-E430G treated mouse on day 0)/(tumor volume of control mouse on day X−tumor volume of control mouse on day 0).

X=the latest day in the period between day 7 to day 25 on which both animals were alive and tumor measurement was performed.

The response values are depicted in Table 5 as well as CD38 mRNA expression. The models that had the highest CD38 mRNA levels also showed the best response. This could also be seen from the graphs in FIG. 20. Thus two weekly doses of IgG1-C-E430G reduced the tumor growth in two out of five tested DLBCL PDX models that had highest CD38 mRNA expression.

TABLE 5
Overview of CD38 mRNA expression and calculated
response value for five DLBCL PDX models. A low
response value indicates tumor regression.

| | CD38 (determined | | Response |
|---|---|---|---|
| | by RNASeq: log2 | Response | calculated |
| Model | (TPM value + 1)) | (ΔT/ΔC) | for day; |
| Ly12638 | 6,427 | −11% | 15 |
| Ly11212 | 6,066 | −2% | 11 |
| Ly13976 | 6,017 | 54% | 13 |
| Ly13693 | 4,796 | 58% | 22 |
| Ly14862 | 0 | 83% | 11 |

### Example 10: IgG1-C-E430G Induces Potent Complement-Mediated Cytotoxicity in Bone Marrow Mononuclear Cells from Newly Diagnosed MM Patients

Bone marrow mononuclear cells (BM-MNC) were isolated by Ficoll-Hypaque density-gradient from full bone marrow aspirates from 3 newly diagnosed MM patients and 1 relapsed/refractory MM patient and frozen at −80° C. until use. On the day of use, BM-MNC were thawed, viable cells were counted and plated in 96-well plates. Cells were incubated with serial dilutions (0.01-10 μg/mL) of IgG1-C-E430G or Darzalex® for 15 min at room temperature on a plate shaker. As negative controls, cells were untreated or were incubated with 10 μg/mL IgG1-b12. As a source of complement, 20% normal human serum was added 45 min prior to FACS measurements, in which absolute numbers of cells were determined using flow cytometric count beads as a constant. To determine the overall percentages lysis, the untreated control wells were used as control values. The percentage multiple myeloma cell lysis was determined relative to controls using the following equation:

% cell lysis=(1−(number of surviving cells in antibody-treated samples/number of surviving cells in untreated controls)×100%

FIGS. 22A and B show that IgG1-C-E430G induced higher levels of lysis in two BM-MNC samples from newly diagnosed MM patients compared to Darzalex®. The maximal lysis induced by IgG1-C-E430G was in the range of 84-90% compared to a maximal lysis in the range of 31-55% induced by Darzalex®. In two other BM-MNC samples, one from a relapsed/refractory MM patient that did not receive Darzalex® as part of prior therapy (FIG. 22C) and one from a newly diagnosed MM patient (FIG. 22D), no induction of CDC was noted with IgG-C-E430G or Darzalex® (FIGS. 22C and D).

### LIST OF REFERENCES

Each reference in this list, or cited elsewhere herein, is hereby specifically incorporated by reference in its entirety.
Antonelli, A., P. Fallahi, et al. (2001). “Anti-CD38 autoimmunity in patients with chronic autoimmune thyroiditis or Graves' disease.” Clin Exp Immunol 126(3): 426-431.
Ausiello, C. M., F. Urbani, et al. (2000). “Functional topography of discrete domains of human CD38.” Tissue Antigens 56(6): 539-547.
Brerski, R. J. and G. Georgiou (2016). “Immunoglobulin isotype knowledge and application to Fc engineering.” Curr Opin Immunol 40: 62-69.
Chatterjee, S., A. Daenthanasanmak, et al. (2018). “CD38-NAD(+)Axis Regulates Immunotherapeutic Anti-Tumor T Cell Response.” Cell Metab 27(1): 85-100 e108.
Cotner, T., M. Hemler, et al. (1981). “Human T cell proteins recognized by rabbit heteroantisera and monoclonal antibodies.” Int J Immunopharmacol 3(3): 255-268.
Dall'Acqua, W. F., K. E. Cook, et al. (2006). “Modulation of the effector functions of a human IgG1 through engineering of its hinge region.” J Immunol 177(2): 1129-1138.
Damle, R. e. a. (1999). “Ig V gene mutation status and CD38 expression as novel prognostic indicators in chronic lymphocytic leukemia.” Blood 94(6): 1840-1847.
de Weers, M., Y. T. Tai, et al. (2011). “Daratumumab, a novel therapeutic human CD38 monoclonal antibody, induces killing of multiple myeloma and other hematological tumors.” J Immunol 186(3): 1840-1848.
Deckert, J., M. C. Wetzel, et al. (2014). “SAR650984, a novel humanized CD38-targeting antibody, demonstrates potent antitumor activity in models of multiple myeloma and other CD38+ hematologic malignancies.” Clin Cancer Res 20(17): 4574-4583.

Deshpande, D. A., T. A. White, et al. (2005). “Altered airway responsiveness in CD38-deficient mice.” Am J Respir Cell Mol Biol 32(2): 149-156.
Desjarlais, J. R. and G. A. Lazar (2011). “Modulation of antibody effector function.” Exp Cell Res 317(9): 1278-1285.
Eissler, N., S. Filosto, et al. (2018). “A best in class anti-CD38 antibody with antitumor and immune-modulatory properties.” AACR annual meeting 2018: Abstract #3812.
Feng X., Zhang L., et al. (2017). “Targeting CD38 Suppresses Induction and Function of T Regulatory Cells to Mitigate Immunosuppression in Multiple Myeloma.” Clin Cancer Res 23:4290-4300.
Ho, H. N., L. E. Hultin, et al. (1993). “Circulating HIV-specific CD8+ cytotoxic T cells express CD38 and HLA-DR antigens.” J Immunol 150(7): 3070-3079.
Kaneko, E. and R. Niwa (2011). “Optimizing therapeutic antibody function: progress with Fc domain engineering.” BioDrugs 25(1): 1-11.
Karakasheva T. A., Waldron T. J., et al., (2015). “CD38-Expressing Myeloid-Derived Suppressor Cells Promote Tumor Growth in a Murine Model of Esophageal Cancer.” Cancer Res 75(19):4074-85.
Kestens, L., G. Vanham, et al. (1992). “Expression of activation antigens, HLA-DR and CD38, on CD8 lymphocytes during HIV-1 infection.” AIDS 6(8): 793-797.
Keyhani, A., Y. O. Huh, et al. (2000). “Increased CD38 expression is associated with favorable prognosis in adult acute leukemia.” Leuk Res 24(2): 153-159.
Konoplev, S., L. J. Medeiros, et al. (2005). “Immunophenotypic profile of lymphoplasmacytic lymphoma/Waldenstrom macroglobulinemia.” Am J Clin Pathol 124(3): 414-420.
Krejcik, J., T. Casneuf, et al. (2016). “Daratumumab depletes CD38+ immune-regulatory cells, promotes T-cell expansion, and skews T-cell repertoire in multiple myeloma.” Blood 128: 384-394.
Krejcik, J., K. A. Frerichs, et al. (2017). “Monocytes and Granulocytes Reduce CD38 Expression Levels on Myeloma Cells in Patients Treated with Daratumumab.” Clin Cancer Res 23(24): 7498-7511.
Lammerts van Bueren, J., D. Jakobs, et al. (2014). “Direct in Vitro Comparison of Daratumumab with Surrogate Analogs of CD38 Antibodies MOR03087, SAR650984 and Ab79.” Blood 124(21): 3474.
Lande, R., F. Urbani, et al. (2002). “CD38 ligation plays a direct role in the induction of IL-1beta, IL-6, and IL-10 secretion in resting human monocytes.” Cell Immunol 220(1): 30-38.
Lee, H. C. and R. Aarhus (1993). “Wide distribution of an enzyme that catalyzes the hydrolysis of cyclic ADP-ribose.” Biochim Biophys Acta 1164(1): 68-74.
Lin, P., R. Owens, et al. (2004). “Flow cytometric immunophenotypic analysis of 306 cases of multiple myeloma.” Am J Clin Pathol 121(4): 482-488.
Malavasi, F., A. Funaro, et al. (1994). “Human CD38: a glycoprotein in search of a function.” Immunol Today 15(3): 95-97.
Mallone, R. and P. C. Perin (2006). “Anti-CD38 autoantibodies in type? diabetes.” Diabetes Metab Res Rev 22(4): 284-294.
Marinov, J., K. Koubek, et al. (1993). “Immunophenotypic Significance of the Lymphoid Cd38 Antigen in Myeloid Blood Malignancies.” Neoplasma 40(6): 355-358.
Morandi F., Horenstein A. L., et al. (2015). “CD56^{bright}CD16⁻ NK Cells Produce Adenosine through a CD38-Mediated Pathway and Act as Regulatory Cells Inhibiting Autologous CD4⁺ T Cell Proliferation.” J Immunol 195:965-972.
Moore, G. L., H. Chen, et al. (2010). “Engineered Fc variant antibodies with enhanced ability to recruit complement and mediate effector functions.” MAbs 2(2): 181-189.
Patton, D. T., Wilson M. D., et al. (2011). “The PI3K p110δ Regulates Expression of CD38 on Regulatory T cells.” PLoS ONE 6(3): 1-8.
Parry-Jones, N., E. Matutes, et al. (2007). “Cytogenetic abnormalities additional to t(11; 14) correlate with clinical features in leukaemic presentation of mantle cell lymphoma, and may influence prognosis: a study of 60 cases by FISH.” Br J Haematol 137(2): 117-124.
Perfetti, V., V. Bellotti, et al. (1994). “AL amyloidosis. Characterization of amyloidogenic cells by anti-idiotypic monoclonal antibodies.” Lab Invest 71(6): 853-861.
Raab, M. S., H. Goldschmidt, et al. (2015). “A phase I/IIa study of the human anti-CD38 antibody MOR202 (MOR03087) in relapsed or refractory multiple myeloma (rrMM).” J Clin Oncol 33: A8574.
Ramaschi, G., M. Torti, et al. (1996). “Expression of cyclic ADP-ribose-synthetizing CD38 molecule on human platelet membrane.” Blood 87(6): 2308-2313.
Roepcke, S., N. Plock, et al. (2018). “Pharmacokinetics and pharmacodynamics of the cytolytic anti-CD38 human monoclonal antibody TAK-079 in monkey—model assisted preparation for the first in human trial.” Pharmacol Res Perspect 6(3): e00402.
Schooten, W. v. (2018). “Multispecific antibodies targeting CD38 and PD-L1 show potent tumor cytotoxicity.” AACR annual meeting 2018: Abstract #5620.
Sondermann, P. and D. E. Szymkowski (2016). “Harnessing Fc receptor biology in the design of therapeutic antibodies.” Curr Opin Immunol 40: 78-87.
Song, A., K. Myojo, et al. (2014). “Evaluation of a fully human monoclonal antibody against multiple influenza A viral strains in mice and a pandemic H1N1 strain in nonhuman primates.” Antiviral Res 111: 60-68.
Suzuki, R., J. Suzumiya, et al. (2004). “Aggressive natural killer-cell leukemia revisited: large granular lymphocyte leukemia of cytotoxic NK cells.” Leukemia 18(4): 763-770.
van de Donk (2018). “Immunomodulatory effects of CD38 targeting antibodies.” Immunology Letters 199:16-22.
van de Donk, N. W., M. L. Janmaat, et al. (2016). “Monoclonal antibodies targeting CD38 in hematological malignancies and beyond.” Immunol Rev 270(1): 95-112.
van de Donk, N. W., H. M. Lokhorst, et al. (2012). “How I treat plasma cell leukemia.” Blood 120(12): 2376-2389.
Wang, L., H. Wang, et al. (2015). “CD38 expression predicts poor prognosis and might be a potential therapy target in extranodal NK/T cell lymphoma, nasal type.” Ann Hematol 94(8): 1381-1388.
Wang, X., M. Mathieu, et al. (2018). “IgG Fc engineering to modulate antibody effector functions.” Protein & Cell 9(1): 63-73.
Zhang, D., A. A. Armstrong, et al. (2017). “Functional optimization of agonistic antibodies to OX40 receptor with novel Fc mutations to promote antibody multimerization.” MAbs 9(7): 1129-1142.
Zocchi, E., L. Franco, et al. (1993). “A single protein immunologically identified as CD38 displays NAD+ glycohydrolase, ADP-ribosyl cyclase and cyclic ADP-ribose hydrolase activities at the outer surface of human erythrocytes.” Biochem Biophys Res Commun 196(3): 1459-1465.
Nijhof et al., Blood 2016; 128(7):959-970.
“Supplemental Methods” to Nijhof et al., 2016.
WO 2006/099875 A1 (Genmab A/S)
WO 2007/042309 A1 (Morphosys AG)
WO 2008/047242 A1 (Sanofi Aventis)
WO 2011/154453 A1 (Genmab A/S)
WO 2012/092612 A1 (Takeda Pharmaceutical)
WO 2013/004842 A2 (Genmab A/S)
WO 2014/108198 A1 (Genmab B.V.)
WO 2016/210223 A1 (Janssen Biotech, Inc.)
WO 2018/031258 A1 (Janssen Biotech, Inc.)

1. An antibody which binds to human CD38, the antibody comprising
(a) an antigen-binding region comprising a VH CDR1 having the sequence as set forth in SEQ ID NO:2, a VH CDR2 having the sequence as set forth in SEQ ID NO:3, a VH CDR3 having the sequence as set forth in SEQ ID NO:4, a VL CDR1 having the sequence as set forth in SEQ ID NO:6, a VL CDR2 having the sequence AAS, and a VL CDR3 having the sequence as set forth in SEQ ID NO:7, and
(b) an Fc region comprising a mutation in one or more amino acid residues selected from the group corresponding to E430, E345 and S440 in a human IgG1 heavy chain, wherein the amino acid residues are numbered according to the EU index.

2-5. (canceled)

6. The antibody according to claim 1, comprising a VH region comprising the sequence of SEQ ID NO:1 and a VL region comprising the sequence of SEQ ID NO:5.

7. The antibody according to claim 1, wherein the mutation in the one or more amino acid residues is selected from the group consisting of E430G, E345K, E430S, E430F, E430T, E345Q, E345R, E345Y, S440Y and S440W.

8-12. (canceled)

13. The antibody according to claim 1, wherein the Fc region is, except for the recited mutation, a human IgG1, IgG2, IgG3 or IgG4 isotype or a mixed isotype thereof.

14-16. (canceled)

17. The antibody according to claim 1, which is a full-length antibody.

18. The antibody according to claim 1, wherein the antibody is, except for the recited mutations, a human antibody.

19-21. (canceled)

22. An antibody variant binding to human CD38, the antibody variant comprising
(a) a heavy chain comprising a VH region comprising a VH CDR1 having the sequence as set forth in SEQ ID NO:2, a VH CDR2 having the sequence as set forth in SEQ ID NO:3, a VH CDR3 having the sequence as set forth in SEQ ID NO:4 and a human IgG1 CH region with a mutation in one or more of E430, E345 and S440, the amino acid residues being numbered according to the EU index;
(b) a light chain comprising a VL region comprising a VL CDR1 having the sequence as set forth in SEQ ID NO:6, a VL CDR2 having the sequence AAS, and a VL CDR3 having the sequence as set forth in SEQ ID NO:7.

23-25. (canceled)

26. The antibody according to claim 1, wherein the Fc region comprises a CH comprising, except for the recited mutation, the sequence of SEQ ID NO:19, SEQ ID NO:20, SEQ ID NO:21, SEQ ID NO:22, SEQ ID NO:23 or SEQ ID NO: 45.

27-28. (canceled)

29. The antibody according to claim 26, wherein Lys (K) at position 447 according to Eu numbering of the CH is deleted.

30. The antibody according to claim 1, wherein the Fc region comprises a CH region comprising an amino acid sequence selected from the group consisting of SEQ ID NO:24 to SEQ ID NO:33 and SEQ ID NO: 46.

31. The antibody according to claim 30, wherein the CH comprises SEQ ID NO:24 or SEQ ID NO: 46, optionally wherein the light chain comprises a CL comprising SEQ ID NO:37.

32-34. (canceled)

35. The antibody variant according to claim 1, which
(a) has an inhibitory effect on the cyclase activity of human CD38;
(b) induces apoptosis in the presence, but not in the absence, of an Fc-cross-linking antibody;
(c) induces CDC, ADCC, antibody-dependent cell-phagocytosis (ADCP), trogocytosis, or any combination thereof, of cells expressing human CD38.

36-42. (canceled)

43. An isolated nucleic acid encoding the antibody according to claim 1.

44. An expression vector comprising the nucleic acid according to claim 43.

45. A nucleic acid encoding a heavy chain of the antibody according to claim 1.

46-47. (canceled)

48. A combination of nucleic acids encoding the antibody according to claim 1.

49. A delivery vehicle comprising the nucleic acid according to claim 45.

50. A delivery vehicle comprising a nucleic acid encoding a light chain of the antibody according to claim 1.

51-54. (canceled)

55. A recombinant host cell which produces the antibody according to claim 1.

56. (canceled)

57. A method of producing the antibody according to claim 1, comprising culturing the recombinant host cell which produces the antibody in a culture medium and under conditions suitable for producing the antibody variant and, optionally, purifying or isolating the antibody variant from the culture medium.

58. A method of increasing at least one effector function of a parent antibody comprising an Fc region and an antigen-binding region binding to CD38, which method comprises introducing into the Fc region a mutation in one or more amino acid residues selected from the group corresponding to E430, E345, and S440 in the Fc region of a human IgG1 heavy chain, wherein the amino acid residues are numbered according to the EU index;
wherein the antigen-binding region comprises a VH CDR1 having the sequence as set forth in SEQ ID NO:2, a VH CDR2 having the sequence as set forth in SEQ ID NO:3, a VH CDR3 having the sequence as set forth in SEQ ID NO:4, a VL CDR1 having the sequence as set forth in SEQ ID NO:6, a VL CDR2 having the sequence AAS, and a VL CDR3 having the sequence as set forth in SEQ ID NO:7.

59. A method of producing a variant of a parent antibody comprising an Fc region and an antigen-binding region binding to CD38, the variant having an increased effector function as compared to the parent antibody, which method comprises
(a) introducing into the Fc region a mutation in one or more amino acid residues selected from the group corresponding to E430, E345, and S440 in the Fc region of a human IgG1 heavy chain to obtain a variant antibody,
(b) selecting any variant antibody having an increased effector function as compared to the parent antibody, and
(c) producing said variant antibody in a recombinant host cell,
wherein the antigen-binding region comprises a VH CDR1 having the sequence as set forth in SEQ ID NO:2, a VH CDR2 having the sequence as set forth in SEQ ID NO:3, a VH CDR3 having the sequence as set forth in SEQ ID NO:4, a VL CDR1 having the sequence as set forth in SEQ ID NO:6, a VL CDR2 having the sequence AAS, and a VL CDR3 having the sequence as set forth in SEQ ID NO:7.

60-68. (canceled)

69. A composition comprising the antibody of claim 1, a nucleic acid encoding the heavy and/or light chain of the antibody, a combination of nucleic acids encoding the heavy chain and light chain of the antibody, an expression vector comprising the nucleic acid, or a host cell comprising the nucleic acid, combination of nucleic acids, or expression vector, and a carrier.

70-71. (canceled)

72. A method of treating a disease involving cells expressing CD38 comprising administering to a subject in need thereof a therapeutically effective amount of the antibody of claim 1, a nucleic acid encoding the heavy and/or light chain of the antibody, a combination of nucleic acids encoding the heavy chain and light chain of the antibody, an expression vector comprising the nucleic acid, or a host cell comprising the nucleic acid, combination of nucleic acids, or expression vector.

73. A method of inducing a CDC-response against a tumor comprising cells expressing CD38 comprising administering to a subject in need thereof a therapeutically effective amount of the antibody of claim 1, a nucleic acid encoding the heavy and/or light chain of the antibody, a combination of nucleic acids encoding the heavy chain and light chain of the antibody, an expression vector comprising the nucleic acid, or a host cell comprising the nucleic acid, combination of nucleic acids, or expression vector.

74. A method of treating a cancer in a subject comprising cells expressing human CD38 comprising administering to a subject in need thereof a therapeutically effective amount of the antibody of claim 1, a nucleic acid encoding the heavy and/or light chain of the antibody, a combination of nucleic acids encoding the heavy chain and light chain of the antibody, an expression vector comprising the nucleic acid, or a host cell comprising the nucleic acid, combination of nucleic acids, or expression vector.

75. The method of claim 74, wherein the cancer is refractory to a prior therapy comprising a CD38 antibody, or wherein the cancer relapsed after a prior therapy comprising a CD38 antibody.

76-78. (canceled)

79. The method according to claim 74, wherein the cancer is a hematological cancer is-selected from the group consisting of multiple-myeloma (MM), chronic lymphocytic leukemia (CLL), acute lymphoblastic leukemia (ALL), acute myelogenous leukemia (adults) (AML), mantle cell lymphoma, follicular lymphoma (FL), and diffuse large B-cell lymphoma (DLBCL).

80-86. (canceled)

87. The method according to claim 74, wherein the cancer is a solid tumor selected from the group consisting of melanoma, lung cancer, squamous non-small cell lung cancer (NSCLC), non-squamous NSCLC, colorectal cancer, prostate cancer, castration-resistant prostate cancer, stomach cancer, ovarian cancer, gastric cancer, liver cancer, pancreatic cancer, thyroid cancer, squamous cell carcinoma of the head and neck, carcinoma of the esophagus or gastrointestinal tract, breast cancer, fallopian tube cancer, brain cancer, urethral cancer, genitourinary cancer, endometrial cancer, cervical cancer, lung adenocarcinoma, renal cell carcinoma (RCC) (e.g., a kidney clear cell carcinoma or a kidney papillary cell carcinoma), mesothelioma, nasopharyngeal carcinoma (NPC), a carcinoma of the esophagus or gastrointestinal tract, and a metastatic lesion of anyone thereof.

88-111. (canceled)

112. A method of treating or preventing rheumatoid arthritis comprising administering to a subject in need thereof a therapeutically effective amount of the antibody of claim 1.

113-115. (canceled)


SEQUENCE LISTING (46 sequences)

SEQ ID NO: 1 - protein, 121 aa, Artificial sequence
  Part of a sequence
QVQLVQSGAEVKKPGSSVKVSCKAFGGTFSSYAISWVRQAPGQGLEWMGRIIRFLGIANYAQKFQGRVTLIADKSTNTAYMELSSLRSEDTAVYYCAGEPGERDPDAVDIWGQGTMVTVSS

SEQ ID NO: 2 - protein, 8 aa, Artificial sequence
  Part of a sequence
GGTFSSYA

SEQ ID NO: 3 - protein, 8 aa, Artificial sequence
  Part of a sequence
IIRFLGIA

SEQ ID NO: 4 - protein, 14 aa, Artificial sequence
  Part of a sequence
AGEPGERDPDAVDI

SEQ ID NO: 5 - protein, 107 aa, Artificial sequence
  Part of a sequence
DIQMTQSPSSLSASVGDRVTITCRASQGIRSWLAWYQQKPEKAPKSLIYAASSLQSGVPSRFSGSGSGTDFTLTISSLQPEDFATYYCQQYNSYPLTFGGGTKVEIK

SEQ ID NO: 6 - protein, 6 aa, Artificial sequence
  Part of a sequence
QGIRSW

SEQ ID NO: 7 - protein, 9 aa, Artificial sequence
  Part of a sequence
QQYNSYPLT

SEQ ID NO: 8 - protein, 122 aa, Artificial sequence [sequence omitted]

SEQ ID NO: 9 - protein, 107 aa, Artificial sequence [sequence omitted]

SEQ ID NO: 10 - protein, 120 aa, Artificial sequence
  Part of a sequence
QVQLVQSGAEVKKPGSSVKVSCKASGGTFSSYAFSWVRQAPGQGLEWMGRVIPFLGIANSAQKFQGRVTITADKSTSTAYMDLSSLRSEDTAVYYCARDDIAALGPFDYWGQGTLVTVSS

SEQ ID NO: 11 - protein, 107 aa, Artificial sequence
  Part of a sequence
DIQMTQSPSSLSASVGDRVTITCRASQGISSWLAWYQQKPEKAPKSLIYAASSLQSGVPSRFSGSGSGTDFTLTISSLQPEDFATYYCQQYNSYPRTFGQGTKVEIK

SEQ ID NO: 12 - protein, 127 aa, Artificial sequence
  Part of a sequence
QVQLVQSGAEVKKPGASVKVSCQASGYRFSNFVIHWVRQAPGQRFEWMGWINPYNGNKEFSAKFQDRVTFTADTSANTAYMELRSLRSADTAVYYCARVGPYSWDDSPQDNYYMDVWGKGTTVIVSS

SEQ ID NO: 13 - protein, 8 aa, Artificial sequence
  Part of a sequence
GYRFSNFV

SEQ ID NO: 14 - protein, 8 aa, Artificial sequence
  Part of a sequence
INPYNGNK

SEQ ID NO: 15 - protein, 20 aa, Artificial sequence
  Part of a sequence
ARVGPYSWDDSPQDNYYMDV

SEQ ID NO: 16 - protein, 108 aa, Artificial sequence
  Part of a sequence
EIVLTQSPGTLSLSPGERATFSCRSSHSIRSRRVAWYQHKPGQAPRLVIHGVSNRASGISDRFSGSGSGTDFTLTITRVEPEDFALYYCQVYGASSYTFGQGTKLERK

SEQ ID NO: 17 - protein, 7 aa, Artificial sequence
  Part of a sequence
HSIRSRR

SEQ ID NO: 18 - protein, 9 aa, Artificial sequence
  Part of a sequence
QVYGASSYT

SEQ ID NO: 19 - protein, 330 aa, Artificial sequence [sequence omitted]

SEQ ID NO: 20 - protein, 330 aa, Artificial sequence
  Part of a sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 330 aa, Artificial sequence
  Part of sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 330 aa, Artificial sequence
  Part of a sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKPVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 330 aa, Artificial sequence
  Part of a sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKPVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEGLHNHYTQKSLSLSPGK

SEQ ID NO: 24 - protein, 330 aa, Artificial sequence
  Part of a sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 25 - protein, 330 aa, Artificial sequence
  Part of a sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHSALHNHYTQKSLSLSPGK

SEQ ID NO: 26 - protein, 330 aa, Artificial sequence
  Part of a sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHFALHNHYTQKSLSLSPGK

SEQ ID NO: 27 - protein, 330 aa, Artificial sequence
  Part of sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHTALHNHYTQKSLSLSPGK

SEQ ID NO: 28 - protein, 330 aa, Artificial sequence
  Part of sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRKPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 330 aa, Artificial sequence
  Part of a sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRQPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 330 aa, Artificial sequence
  Part of sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRRPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 330 aa, Artificial sequence
  Part of a sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPRYPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 330 aa, Artificial sequence
  Part of sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKWLSLSPGK

SEQ ID NO: 33 - protein, 330 aa, Artificial sequence
  Part of sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKYLSLSPGK

SEQ ID NO: 34 - protein, 326 aa, Artificial sequence
  Part of sequence
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 35 - protein, 377 aa, Artificial sequence
  Part of sequence
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 36 - protein, 327 aa, Artificial sequence
  Part of sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 37 - protein, 107 aa, Artificial sequence [sequence omitted]

SEQ ID NO: 38 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 39 - protein, 262 aa, Artificial sequence
  Tagged sequence
HHHHHHRWRQTWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 40 - protein, 8 aa, Artificial sequence
  Part of sequence
  X (5)..(5)
  MISC_FEATURE (5)..(5): X is Ser or Arg
GGTFXSYA

SEQ ID NO: 41 - protein, 8 aa, Artificial sequence
  Part of sequence
  MISC_FEATURE (3)..(3): X is Arg or Val
  MISC_FEATURE (7)..(7): X is Ile or Lys
  MISC_FEATURE (8)..(8): X is Ala, Tyr or Val
  MISC_FEATURE (8)..(8): X is Ala, Thr or Val
IIXFLGXX

SEQ ID NO: 42 - protein, 14 aa, Artificial sequence
  Part of sequence
  MISC_FEATURE (1)..(1): X is Ala or Thr
  MISC_FEATURE (6)..(6): X is Glu, Asp or Ala
  MISC_FEATURE (12)..(12): X is Val or Phe
XGEPGXRDPDAXDI

SEQ ID NO: 43 - protein, 6 aa, Artificial sequence
  Part of sequence
QGIRSW

SEQ ID NO: 44 - protein, 9 aa, Artificial sequence
  Part of sequence
  MISC_FEATURE (5)..(5): X is Ser or Asn
QQYNXYPLT

SEQ ID NO: 45 - protein, 329 aa, Artificial sequence
  Part of sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 46 - protein, 329 aa, Artificial sequence
  Part of sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLSPG